(19) United States
(12) Patent Application Publication
(10) Pub. No.: US 2018/0119141 A1
(43) Pub. Date: May 3, 2018

(54) CRISPR/CAS GLOBAL REGULATOR SCREENING PLATFORM

(71) Applicant: Massachusetts Institute of Technology, Cambridge, MA (US)

(72) Inventors: Ying-Chou Chen, Waltham, MA (US); Fahim Farzadfard, Boston, MA (US); Timothy Kuan-Ta Lu, Cambridge, MA (US)

(73) Assignee: Massachusetts Institute of Technology, Cambridge, MA (US)

(21) Appl. No.: 15/411,255

(22) Filed: Jan. 20, 2017

Related U.S. Application Data
(60) Provisional application No. 62/414,277, filed on Oct. 28, 2016.

(51) Int. Cl. C12N 15/113; G01N 33/50; C12N 15/85

(52) U.S. Cl. CPC C12N 15/113; C12N 15/85; G01N 33/5014

(57) ABSTRACT

Provided herein are methods for identifying genetic networks and methods of treating neurodegenerative disorders associated with α-synuclein dysfunction.

### RELATED APPLICATION

This application claims the benefit under 35 U.S.C. § 119(e) of U.S. provisional application No. 62/414,277, filed Oct. 28, 2016, which is incorporated by reference herein in its entirety.

### GOVERNMENT FUNDING

This invention was made with government support under Grant No. P50 GM098792 awarded by the National Institutes of Health. The government has certain rights in the invention.

### FIELD OF INVENTION

This invention relates to methods of identifying genetic networks using a CRISPR/Cas screening platform and methods of treating neurodegenerative disorders associated with α-synuclein dysfunction in a subject.

### BACKGROUND

The systematic perturbation of transcriptional networks enables the elucidation of gene functions and regulatory networks that underlie biological processes. Transcription perturbations introduced by artificial transcription factors, such as CRISPR-Cas9-based transcription factors (crisprTFs), enable bi-directional gene activation and repression in eukaryotic systems. However, current methods rely on guide RNAs (gRNAs) that are designed to target individual genes (or a limited number of targeted genes), while minimizing off-target effects.

### SUMMARY

Aspects of the present disclosure provide methods for treating a neurodegenerative disorder associated with α-synuclein dysfunction comprising administering to a subject having a disorder associated with α-synuclein dysfunction a therapeutically effective amount of an agent that enhances expression and/or activity of a human homolog of one or more genes set forth in Table 1. In some embodiments, if the agent enhances expression of one gene set forth in Table 1, the gene is not heat shock protein (HSP)30, HSP31. HSP32, HSP33, HSP34, UBC8, or YGR130C. In some embodiments, if the agent enhances expression of one gene set forth in Table 1, the gene is not HSP30, HSP31, UBC8, YGR130C or YPL123C (RNY1).

In some embodiments, the gene is selected from the group consisting of YBL086C, YBR056W, SAF1, DAD1, ARX1, ARP10, PET117, STF2, SPL2, YJL144W, TRX1, SRN2, SHH4, ECM19, SNO4, SIS1, DBP2, VHS3, HSP32, GGA1, TIM9, HSP42, YER121W, YGL258W-A, CPD1, YLR149C, NCE103, YOL114C, OXR1, URA7, YDL199C, YKL100C, YMR244W, ATO2, PHM7, PNS1, and YPL247C. In some embodiments, the human homolog is HSPB1, HSPB3, HSPB6, HSPB7, HSPB8, HSPB9, CRYAA, CRYAB, DNAJB1-B9, GGA1, GGA2, GGA3, TOM1, TOM1L1, TOM1L2, WDFY1, WDFY2. ALS2, RCC1, TXN, TXNDC2, TXNDC8, TIMM9, OXR1, NCOA7, TLDC2, PA2G4, XPNPEP1, XPNPEP2, SDHD, DDX17, DDX41, DDX43, DDX5, DDX53, DDX59, PPCDC, ICT1, CTPS1, CTPS2, HM13, SPPL2A, SPPL2C, SPPL3, TMEM63 (A-C), SLC44 (A1-A5), DCAF7, SERBP1, or HABP4. In some embodiments, at least two agents that enhance expression and/or activity of TIMM9 and TXN are administered.

In some embodiments, the agent is a small molecule, protein, or a nucleic acid. In some embodiments, the agent is a gRNA, siRNA, miRNA, shRNA, or a nucleic acid encoding a gene. In some embodiments, the agent is a nucleic acid encoding a gene, which is a human homolog of one or more of the genes set forth in Table 1. In some embodiments, the agent is a gRNA and comprises a nucleotide sequence provided by SEQ ID NO: 1 (gRNA 9-1) or SEQ ID NO: 2 (gRNA 6-3). In some embodiments, the agent is encoded on a vector.

In some embodiments, the agent is administered with a pharmaceutically acceptable excipient. In some embodiments, the agent is administered in one dose. In some embodiments, the agent is administered in multiple doses. In some embodiments, the agent is administered orally, intravenously, intraperitoneally, topically, subcutaneously, intracranially, intrathecally, or by inhalation.

In some embodiments, the disorder associated with α-synuclein dysfunction is Parkinson's disease, Lewy body variant of Alzheimer's disease, diffuse Lewy body disease, dementia with Lewy bodies, multiple system atrophy, or neurodegeneration with brain iron accumulation type I.

Other aspects provide nucleic acids comprising the nucleotide sequence provided by SEQ ID NO: 1 (gRNA 9-1) or SEQ ID NO: 2 (gRNA 6-3). Yet other aspects provide vectors encoding any of the nucleic acids described herein.

Aspects of the present disclosure provide methods for identifying a genetic network involved in regulating a cellular response, comprising (i) expressing in a population of cells a plurality of randomized guide RNAs and a CRISPR protein; (ii) culturing the population of cells under conditions that induce the cellular response; (iii) isolating a subpopulation of cells having an altered readout of the cellular response from the population of cells; and (iv) identifying a randomized guide RNA present in the cells isolated in (iii) as a guide RNA that regulates a transcriptional network involved in the cellular response. In some embodiments, the cellular response is α-synuclein toxicity. In some embodiments, the altered readout of the cellular response is reduced α-synuclein toxicity.

In some embodiments, the randomized guide RNA comprises a plurality of nucleotides, wherein the content of guanine and cytosine nucleotides in the randomized guide RNA is between 50% and 70%.

Also provided herein are methods for identifying a transcriptional network involved in suppression of α-synuclein toxicity, comprising (i) expressing in a population of cells a plurality of randomized guide RNAs and a CRISPR-Cas transcription factor; (ii) culturing the population of cells under conditions of α-synuclein toxicity; (iii) isolating a subpopulation of cells having suppressed α-synuclein toxicity from the population of cells; and (iv) identifying a randomized guide RNAs present in the cells isolated in (iii) as a guide RNA that regulates a transcriptional network involved in suppression of α-synuclein toxicity.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. For purposes of clarity, not every component may be labeled in every drawing. In the drawings:

FIGS. 1A-1C show identification of genetic modifiers of αSyn toxicity in S. cerevisiae identified using randomized gRNA/crisprTF screens. FIG. 1A presents a schematic illustration of engineered screening yeast strain expressing αSyn and crisprTF (left) and the strategy used for building randomized gRNA library (right). FIG. 1B shows sequences of the two identified gRNAs (designated as gRNA 6-3 (SEQ ID NO: 2) and gRNA 9-1 (SEQ ID NO: 1)) that were found to suppress αSyn-mediated toxicity. Saturated cultures were diluted in 5-fold serial dilutions and spotted on Scm (Synthetic complete media)−Ura (Uracil)+Glucose+Dox (Doxycycline) plates to quantify total number of viable cells and Scm−Ura+Galactose+Dox plates to score cell viability upon αSyn induction (on galactose). gRNA 9-1 was found to be a strong suppressor of αSyn toxicity, while gRNA 6-3 was found to be a moderate suppressor. Both gRNAs suppressed αSyn toxicity better than the negative control (empty vector), and the suppression level was independent of gRNA plasmid copy number. FIG. 1C shows transcriptomic analysis of the S. cerevisiae strain harboring gRNA 9-1 compared to the reference strain (S. cerevisiae strain with no gRNA) represented as a volcano plot (fold change vs. statistical significance). A list of differentially expressed genes is provided in the Table 1.

FIGS. 2A-2C show that overexpressing genes identified from the gRNA 9-1/crisprTF screen rescue αSyn-associated cellular defects in yeast. FIG. 2A shows survival upon αSyn induction of S. cerevisiae harboring gRNA 9-1 (‘gRNA 9-1’) compared to cells expressing the empty vector (‘Vector’) and those overexpressing HSP31-34 (heat shock proteins) (top panels), as well as top-ranked αSyn suppressors identified in this screen (bottom panels). UBP3, a known strong αSyn suppressor, was used as a positive control. FIG. 2B shows quantification of αSyn-YFP foci in the S. cerevisiae strain harboring no gRNA, gRNA9-1, or plasmids that overexpress the indicated genes. Cytoplasmic YFP foci represent αSyn aggregates produced as a result of defects in vesicular trafficking. Cells expressing crisprTF and gRNA 9-1 robustly inhibited αSyn aggregates, as evidenced by the absence of cytoplasmic YFP foci in these samples. Cells overexpressing UBP3 were used as a positive control in this assay. Data were presented as mean±SEM of three biological replicates. FIG. 2C shows representative micrographs of αSyn-expressing cells shown in FIG. 2B. Bar=10 μm.

FIGS. 3A-3E show the effects of expressing human homologs of yeast αSyn-toxicity suppressors in a human neuronal PD model. FIG. 3A shows a schematic representation of the experimental procedure used for testing the human homologs of the identified yeast αSyn suppressors in differentiated neuronal cell lines. Different constructs expressing individual genes were transfected into SH-SY5Y neuroblastoma cell line via transient transfection to examine their ability to protect against αSyn toxicity. αSyn expression was induced by removal of Dox from the media, and retinoic acid (RA) treatment was used for neuronal differentiation over the course of a six-days period. The cell death inhibitor zVAD and toxin MPP+ were applied in control experiments. FIG. 3B shows viability of differentiated cell lines overexpressing αSyn and the indicated constructs (left panel), as determined by CellTiter-Glo luminescent assay. Expression of individual genes did not significantly affect cell survival of differentiated cells in the absence of αSyn induction (right panel). Constructs expressing human DJ-1 (homolog of yeast SNO4/HSP34 and HSP32), GGA1 (GGA1), ALS2 (SAF1), and DNAJB1 (SIS1) were tested. Bcl-xL, which is known to protect apoptotic neuronal death, was used a positive control (Dietz et al. J. Neurochem. (2008) 104: 757-765). FIG. 3C shows the percentage of dead cells with αSyn induction (white bars) and without αSyn induction (black bars), as quantitated by FITC-Annexin V staining followed by flow cytometry. Overexpression of DJ-1 and ALS2 was compared with the cell death inhibitor zVAD. FIG. 3D shows survival of cells expressing human TXN (homolog of yeast TRX1) and TIMM9 (TIM9) individually or together to test for synergistic effects on suppressing αSyn toxicity. The left panel shows with αSyn induction, and the right panel shows without αSyn induction. FIG. 3E shows that overexpression of DJ-1, TIMM9, or TXN+TIMM9 did not protect against MPP+toxicity, in contrast with Bcl-xL overexpression. Transfected and differentiated cells were treated with 6 mM MPP+ and then tested for cell viability 48 hours later. All data were presented as mean±SEM of triplicate sets. *p<0.05, **p<0.01, ***p<0.001, ****p<0.0001; ns, not significant.

FIGS. 4A-4D show lentiviral expression of human DJ-1, TXN, and TIMM9 protects against αSyn-associated toxicity in a neuronal model of Parkinson's disease (PD). FIG. 4A presents a schematic representation of the experimental procedure in which the human homologs of yeast αSyn-toxicity suppressors were stably expressed via lentiviral vectors six days before retinoic acid (RA) treatment and αSyn induction. FIG. 4B shows that overexpression of DJ-1 or TXN+TIMM9 significantly increased neuronal viability in the presence of αSyn induction. The 2A peptide sequence (P2A) was used to achieve the simultaneous expression of multiple genes from a single promoter. Bars in each set, left to right: EGFP, DJ-1-P2A-EGFP, TXN-P2A-EGFP.TIMM9-P2A-EGFP. TIMM9-P2A-EGFP-P2A-EGFP, and non-infection. FIG. 4C shows that TXN and TIMM9 work synergistically to protect neural cells from αSyn toxicity based on Highest Single Agent (Max(E_{TXN}, E_{TIMM9})) (Borisy et al. Proc. Natl. Acad. Sci. USA (2003) 100: 7977-7982), Linear Interaction Effect (E_{TXN}+E_{TIMM9}) (Slinker J. Mol. Cell. Cardiol. (1998) 30:723-731), and Bliss Independence ((E_{TXN}+E_{TIMM9}−E_{TIMM9}) (Greco et al. Pharmacol. Rev. (1995) 47: 331-385) models (dashed lines). The αSyn toxicity suppression effect observed when TXN+TIMM9 were over-expressed was greater than the threshold values obtained from these models. FIG. 4D presents representative micrographs showing neuronal morphology and cell density of cells transfected with lentiviral vectors over-expressing the indicated human genes. Bar=400 μm. All data were presented as mean±SEM, n=6. *p<0.05, **p<0.01. ***p<0.001, ****p<0.0001.

FIG. 5 shows growth profiles of the parental S. cerevisiae strain and S. cerevisiae strains used in the screen. Growth profiles of the αSyn-expressing parental yeast strain (black lines) as well as strains expressing both αSyn and crisprTF (dCas9-VP64) (gray lines) were determined in glucose and galactose media, and in the presence of Dox for dCas9-VP64 induction. The cells in this assay did not contain gRNAs. Cell density was measured by OD₆₀₀ at the indicated time points. Parental S. cerevisiae strains and screening strains exhibited similar growth profiles in glucose media, and both strains showed severe growth defects upon αSyn induction in galactose media, suggesting that expression of dCas9-VP64 by itself did not affect αSyn-mediated toxicity. Error bars represent the standard error of three independent biological replicates.

FIG. 6 shows that gRNA-mediated suppression of αSyn toxicity depends on the presence of dCas9-VP64. Suppression of αSyn toxicity in the absence of the crisprTF was assessed by expressing gRNA 6-3 or gRNA 9-1 in the αSyn-expressing parental yeast strain, which does not express dCas9-VP64. Neither gRNA 6-3 nor gRNA 9-1 was able to suppress αSyn toxicity. These results, along with the data presented in FIG. 1B, demonstrate that the αSyn toxicity protective effect of gRNA 6-3 and gRNA 9-1 depends on the expression of dCas9-VP64.

FIGS. 7A-7C show the effect of gRNA 9-1/crisprTF on αSyn expression level and suppression of αSyn toxicity. FIG. 7A shows the expression level of GAL4, SNCA (αSyn) and ACT1 following RT-PCR using gene-specific primers. Overnight cultures of the yeast strains harboring no gRNA (‘Vector’) or gRNA 9-1 (‘gRNA 9-1’) were grown in glucose and galactose media for 3 or 6 hours. Total RNA was extracted from these samples, and the gene expression analyzed. Representative data from two independent experiments are shown. FIG. 7B shows quantitative real-time PCR performed with the same gene-specific primers in FIG. 7A with expression levels normalized to expression of the genes in glucose cultures (6 hours, n=4). Primer sequences are provided in Table 6. FIG. 7C shows an alignment of gRNA 9-1 and one of the predicted binding sites of gRNA 9-1 located within the GAL4 open reading frame (Table 2). To investigate the effect of gRNA 9-1/crisprTF on GALA expression and exclude the possibility that the αSyn toxicity suppressive effect of gRNA 9-1 was mediated by repressing GAL4 expression (which acts as an activator of the GAL promoter that drives αSyn expression), the predicted gRNA 9-1 binding site in GAL4 was removed by substituting six synonymous codons from Leu49 to Leu54. The modified GAL4 is designated as GAL4*. As shown in the growth assays, compared with the vector control, gRNA 9-1 expression consistently suppressed αSyn toxicity in two independent S. cerevisiae strains expressing the GAL4* modification, indicating that the suppression of αSyn toxicity mediated by gRNA 9-1/crisprTF was independent of the interaction between GAL4 and gRNA 9-1. From top to bottom, the sequences in this figure are: SEQ ID NO: 3. SEQ ID NO: 4, SEQ ID NO: 5, SEQ ID NO: 6, SEQ ID NO: 170, SEQ ID NO: 4, SEQ ID NO: 171, and SEQ ID NO: 6.

FIG. 8 shows the systematic over-expression of genes modulated by gRNA 9-1 and evaluation of the effects of over-expressing each gene on αSyn toxicity. Plasmids containing each of the indicated genes that are predicted to be modulated by gRNA 9-1 were obtained from yeast ORF library (Open Biosystems Yeast ORF Collection) and transformed into the screening S. cerevisiae strain. Cells expressing individual genes were spotted onto galactose-containing plates and scored for the suppression of αSyn toxicity in comparison to cells expressing dCas9-VP64 and gRNA 9-1 (“gRNA 9-1”), as well as those expressing dCas9-VP64 and vector control (“Vector”). UBP3 (a known suppressor of αSyn toxicity) was used as a positive control. A complete list of differentially expressed genes and annotations as well as associated scores are presented in Table 1.

FIG. 9 shows the examination of αSyn toxicity suppression by a set of over-expressed genes randomly selected from yeast ORF library. Thirty-four yeast genes were randomly chosen from yeast ORF library (Open Biosystems Yeast ORF Collection) and transformed into the screening S. cerevisiae strain. Cell survival in the presence of αSyn induction was measured by a spotting assay and compared to survival of cells expressing dCas9-VP64 and gRNA 9-1 (‘gRNA 9-1’; scored as 5) as well as those expressing dCas9-VP64 and vector control (‘Vector’; scored as 1). Only five genes (YJL110C, YOR116C, YNL065W, YNL135C, and YKL194C) out of 34 genes scored greater than or equal to 2. A complete list of genes and annotations as well as associated scores are presented in Table 5.

FIGS. 10A-10B show an investigation of the effect of over-expression of candidate genes on αSyn expression level in yeast. FIG. 10A shows the expression level of αSyn-YFP as quantified by flow cytometry (using LSR Fortessa II flow cytometer equipped with 488/FITC laser/filter set) and normalized to the non-induced control. Briefly, overnight cultures of screening S. cerevisiae strain overexpressing the indicated genes were induced in Scm−Ura+galactose+Dox for 18 hours. Data are presented as mean±SEM of three biological replicates. FIG. 10B shows the expression of αSyn-YFP and proteins encoded by the indicated genes as further validated by Western blotting of whole cell lysates of the S. cerevisiae strains.

FIGS. 11A and 11B show inducible expression of αSyn in the human neural model of Parkinson's disease (PD). FIG. 11A shows expression of αSyn and ß-gal (non-toxic negative control) was induced in human SH-SY5Y neuroblastoma cells by removal of Dox from media. αSyn-expressing cells significantly lost viability at the 6th day post-differentiation (retinoic acid (RA) treatment). FIG. 11B presents representative images showing retraction of neuritic processes, membrane blebbing, and cell death in αSyn-expressing cells (−Dox condition). Bar=10 μm.

FIGS. 12A-12C show an investigation of the effect of over-expression of TRX and TIM family proteins on αSyn toxicity in yeast. FIG. 12A shows yeast TRX and TIM family proteins function together to protect mitochondria from oxidative stresses (Durigon et al. EMBO Reports (2012) 13: 916-922). Genes in the TRX and TIM families were identified in gRNA 9-1 expression profiling. Cells harboring individual genes from the TRX family (TRX1 and TRX2) and TIM family (TIM8, TIM9, and TIM10) were over-expressed in the screening S. cerevisiae strain to test suppression of αSyn toxicity. All these proteins strongly suppressed αSyn toxicity when over-expressed. Synergistic protective effects were not observed in yeast assays when TRX1 and TIM9 were co-expressed, likely due to the strong αSyn toxicity suppression achieved by over-expression of each of the individual genes. FIG. 12B shows representative micrographs of αSyn-YFP foci in S. cerevisiae cells overexpressing TRX1, TIM9 or both TRX1 and TIM9. Bar=10 μm. FIG. 12C shows αSyn-YFP foci in S. cerevisiae strains co-expressing other gene pairs (SNO4+GGA1, SNO4+HSP32, and SNO4+TIM9). None of the indicated gene pairs demonstrated synergistic αSyn toxicity protection as compared to single gene expression.

FIGS. 13A and 13B show the design and optimization of MPP+ treatment in the neuronal toxicity assay. FIG. 13A presents a schematic of the experimental procedure used to study the effect of MPP+, a known inducer of neural cell death, on differentiated SH-SY5Y cells. FIG. 13B shows the results from a series of titration treatments to identify minimal concentration of MPP+ that resulted in maximal toxicity. Cells were treated with different concentrations of MPP+ for 48 hours, and cell viability was measured by CellTiter-Glo luminescent assay and normalized to the non-MPP+ treatment (n=3). 6 mM MPP+ was found to be the optimal concentration for maximal toxicity, and therefore was used in the survival assay.

### DETAILED DESCRIPTION

Conventional methods of CRISPR-based screening strategies rely on targeted gene activation or repression while minimizing or avoiding off-target effects. Many of these methods involve designing guide RNAs (gRNAs) that hybridize (are complementary) to one target locus and minimize or avoid mismatches between the gRNA and the target locus. In contrast, the methods described herein are based, at least in part, on screening methods using randomized (fully randomized or pseudo-randomized) gRNAs, which provide promiscuity of binding of gRNAs to target sequences to modulate expression of multiple genes that may contribute to a cellular process.

The methods described herein provide global perturbations of genetic networks, which are difficult to elucidate using traditional single- or multiple-gene perturbations. The methods described herein allow for the identification of genes involved in cellular processes involving multi-layered regulatory networks, such as those associated with complex human diseases or disorders (e.g., neurodegenerative disorders associated with α-synuclein dysfunction). Without wishing to be bound by any theory, such genes may encode proteins that are involved in processes/pathways involved in the development and/or pathology of the disorder, and therefore represent targets for treatment methods.

It is generally thought in the art that mismatches between a gRNA and a nucleic acid to which is hybridizes will abrogate activity of the CRISPR protein (e.g. gene activation or repression). However, it was surprisingly found that the mismatches between the gRNA and the nucleic acid to which it hybridized allowed for the identification of genetic networks involved in suppressing α-synuclein in yeast cells. Such methods may be used to identify networks involved in other complex multilayers processes.

Also provided herein are methods of treating neurodegenerative disorders associated with α-synuclein dysfunction by administering an agent that enhances the expression and/or activity of a human homolog of one or more genes provided in Table 1.

### Identification of Target Genes

The methods described herein are based, at least in part, on the identification of genes of S. cerevisiae that when over-expressed in a cell provided a protective effect and suppressed toxicity (cell death) induced by α-synuclein dysfunction. As described in the Example, human homologs of the S. cerevisiae genes that were identified as conferring a protective effect were validated and found to also provide protective effects when the expression and/or activity was enhanced. As described herein, enhancing the expression and/or activity of human homologs of one or more genes provided in Table 1 would be expected to confer protective and beneficial effects when administered to a subject. Accordingly, administration of agents that enhance the expression and/or activity of human homologs of one or more genes provided in Table 1 may be administered to a subject to treat neurodegenerative disorders associated with α-synuclein dysfunction. Any one of more gene for which expression and/or activity are enhanced by an agent may be referred to as a “target gene.”

As used herein, a “human homolog” of a yeast gene, such as a S. cerevisiae gene provided in Table 1, refers to a human gene that is predicted to be functionally conserved to a corresponding yeast gene. Homologous genes are genes in at least two different organisms, such as a yeast and a subject as described herein (e.g., a human subject), that are thought to have descended from a common ancestral gene. Any method known in the art may be used to identify a human homolog of a yeast gene, including web-based algorithms.

In some embodiments, the agent enhances the expression and/or activity of a human homolog of one or more (e.g., 1, 2, 3, 4, 5, 6, 7, 8, 9, 10, or more) genes provided in Table 1. When the agent administered in the methods described herein includes one gene, the gene is not HSP30. HSP31, HSP32, HSP33, HSP34, UBC8, or YGR130C. In some embodiments, the agent enhances expression and/or activity of a human homolog of at least 1, 2, 3, 4, 5, 6, 7, 8, 9, 10, or more genes provided in Table 1. In some embodiments, the agent enhances the expression and/or activity of a human homolog of 1-10, 1-20, 1-30, 2-20, 5-10, 5-15, 5-25, 10-20, 5-30, 10-40, 20-50, 30-60, or 25-75 genes provided in Table 1.

As described in the Example, it was unexpectedly found that several genes identified as providing a protective effect to α-synuclein toxicity encode proteins that were related (e.g., belonging to the same protein family) or involved in related cellular processes. For example, the genes that when over-expressed conferred a protective effect to α-synuclein toxicity were enriched for genes belonging to Gene Ontology (GO) categories including protein quality control, ER/Golgi trafficking, lipid metabolism, mitochondrial function, and stress responses. In some embodiments, the agent enhances the activity of a human homology of at least one gene encoding a protein that is predicted to function in protein quality control. ER/Golgi trafficking, lipid metabolism, mitochondrial function, and stress responses.

In some embodiments, the agent enhances the expression and/or activity of a human homolog of a gene is selected YBL086C, YBR056W, SAF1, DAD1, ARX1, ARP10, PET117, STF2, SPL2, YJL144W, TRX1, SRN2, SHH4, ECM19, SNO4, SIS1, DBP2, VHS3, HSP32, GGA1, TIM9, HSP42, YER121W, YGL258W-A, CPD1, YLR149C, NCE103, YOL114C, OXR1, URA7, YDL199C, YKL100C, YMR244W, ATO2, PHM7, PNS1, and YPL247C. In some embodiments, the agent enhances the expression and/or activity of a human homolog of each of the genes: YBL086C, YBR056W, SAF1, DAD1, ARX1, ARP10, PET117, STF2, SPL2, YJL144W, TRX1, SRN2, SHH4, ECM19, SNO4, SIS1, DBP2, VHS3, HSP32, GGA1, TIM9, HSP42, YER21 W, YGL258W-A, CPD1, YLR149C, NCE103, YOL114C, OXR1, URA7, YDL199C. YKL100C, YMR244W, ATO2, PHM7. PNS1, and YPL247C.

In some embodiments, the human homolog is HSPB1, HSPB3, HSPB6, HSPB7, HSPB8, HSPB9, CRYAA, CRYAB, DNAJB1-B9, GGA1, GGA2, GGA3, TOM1, TOM1L1, TOM1L2, WDFY1, WDFY2, ALS2, RCC1, TXN, TXNDC2, TXNDC8, TIMM9, OXR1, NCOA7, TLDC2, PA2G4, XPNPEP1, XPNPEP2, SDHD, DDX17, DDX41, DDX43, DDX5, DDX53, DDX59, PPCDC, ICT1, CTPS1, CTPS2, HM13, SPPL2A, SPPL2C, SPPL3, TMEM63 (A-C), SLC44 (A1-A5), DCAF7, SERBP1, or HABP4.

As described herein, it was also found that enhancing the activity and/or expression of more than one gene may provide synergistic effects. For example, enhanced expression and/or activity of TXN and TIMM9, human homologs of TRX1 and TIM9, respectively, resulted in a synergistic effect with an enhanced suppression of α-synuclein toxicity, as compared to the effects observed when the expression and/or activity of TXN and TIMM9 were enhanced alone. As used herein, the term “synergistic effect” or “synergy” refers to a combination that provides an observed effect that is greater than the expected sum of the effect of each of the individual components. A combination, such as a combination of genes (e.g., human homologs of genes provided in Table 1) for which expression and/or activity is enhanced may be identified as a synergistic combination by any means known in the art, such as by Highest Single Agent, Linear Interaction Effect, and Bliss Independence models. See, e.g., Borisy et al., Proc Natl Acad Sci USA (2003) 100: 7977-7982; Slinker, J. Mol & Cell. Cardio. (1998) 30: 723-731; and Greco et al. Pharmacol. Rev. (1995) 47: 331-385.

TABLE 1
Genes regulated by gRNA 9-1 that suppress α-synuclein toxicity when overexpressed

| | | | | αSyn | | |
|---|---|---|---|---|---|---|
| | | | | Suppression | | |
| | | | | Score | | |
| | | Fold change | | (when over- | | |
| | | (log2(gRNA 9 | | expressed | Human | |
| Systematic | Standard | RPKM/Ref | p- | from a | Homolog and | |
| Name | Name | RPKM)) | value | plasmid) | Ortholog | Description |
| YBL086C | | 1.1009 | 0.1 | 4.5 | | Protein of unknown function; green fluorescent |
| | | | | | | protein (GFP)-fusion protein localizes to the cell |
| | | | | | | periphery |
| YBR056W | | 1.06549 | 0.1 | 4.5 | | Putative glycoside hydrolase of the mitochondrial |
| | | | | | | intermembrane space |
| YBR280C | SAF1 | 1.18006 | 0.1 | 4.5 | ALS2, RCC1 | F-Box protein involved in proteasome-dependent |
| | | | | | | degradation of Aah1p; involved in proteasome- |
| | | | | | | dependent degradation of Aah1p during entry of |
| | | | | | | cells into quiescence; interacts with Skp1 |
| YDR016C | DAD1 | 1.10875 | 0.1 | 4.5 | | Essential subunit of the Dam1 complex (aka DASH |
| | | | | | | complex); complex couples kinetochores to the |
| | | | | | | force produced by MT depolymerization thereby |
| | | | | | | aiding in chromosome segregation; is transferred to |
| | | | | | | the kinetochore prior to mitosis |
| YDR101C | ARX1 | −1.04375 | 0.1 | 4.5 | PA2G4, | Nuclear export factor for the ribosomal pre-60S |
| | | | | | XPNPEP1, | subunit; shuttling factor which directly binds FG |
| | | | | | XPNPEP2 | rich nucleoporins and facilities translocation |
| | | | | | | through the nuclear pore complex; interacts directly |
| | | | | | | with Alb1p; responsible for Tif6p recycling defects |
| | | | | | | in the absence of Rei1; associated with the |
| | | | | | | ribosomal export complex |
| YDR106W | ARP10 | 1.16556 | 0.1 | 4.5 | | Component of the dynactin complex; localized to |
| | | | | | | the pointed end of the Arp1p filament; may regulate |
| | | | | | | membrane association of the complex |
| YER058W | PET117 | 1.23464 | 0.1 | 4.5 | | Protein required for assembly of cytochrome c |
| | | | | | | oxidase |
| YGR008C | STF2 | 2.00423 | 0.1 | 4.5 | HABP4, | Protein involved in resistance to desiccation stress; |
| | | | | | SERBP1 | Stf2p exhibits antioxidant properties, and its |
| | | | | | | overexpression prevents ROS accumulation and |
| | | | | | | apoptosis; binds to F0 sector of mitochondrial F1F0 |
| | | | | | | ATPase in vitro and may modulate the inhibitory |
| | | | | | | action of Inh1p and Stf1p; protein abundance |
| | | | | | | increases in response to DNA replication stress; |
| | | | | | | STF2 has a paralog, TMA10, that arose from the |
| | | | | | | whole genome duplication |
| YHR136C | SPL2 | −1.26719 | 0.1 | 4.5 | | Protein with similarity to cyclin-dependent kinase |
| | | | | | | inhibitors; downregulates low-affinity phosphate |
| | | | | | | transport during phosphate limitation by targeting |
| | | | | | | Pho87p to the vacuole; upstream region harbors |
| | | | | | | putative hypoxia response element (HRE) cluster; |
| | | | | | | overproduction suppresses a plc1 null mutation; |
| | | | | | | promoter shows an increase in Snf2p occupancy |
| | | | | | | after heat shock; GFP-fusion protein localizes to the |
| | | | | | | cytoplasm |
| YJL144W | | 1.14196 | 0.1 | 4.5 | | Cytoplasmic hydrophilin essential in desiccation- |
| | | | | | | rehydration process; expression induced by osmotic |
| | | | | | | stress, starvation and during stationary phase; |
| | | | | | | protein abundance increases in response to DNA |
| | | | | | | replication stress |
| YLR043C | TRX1 | 1.07168 | 0.1 | 4.5 | TXN, | Cytoplasmic thioredoxin isoenzyme; part of |
| | | | | | TXNDC2, | thioredoxin system which protects cells against |
| | | | | | TXNDC8 | oxidative and reductive stress; forms LMA1 |
| | | | | | | complex with Pbi2p; acts as a cofactor for Tsa1p; |
| | | | | | | required for ER-Golgi transport and vacuole |
| | | | | | | inheritance; with Trx2p, facilitates mitochondrial |
| | | | | | | import of small Tims Tim9p, Tim10p, Tim13p by |
| | | | | | | maintaining them in reduced form; abundance |
| | | | | | | increases under DNA replication stress; TRX1 has a |
| | | | | | | paralog, TRX2, that arose from the whole genome |
| | | | | | | duplication |
| YLR119W | SRN2 | 1.03094 | 0.1 | 4.5 | | Component of the ESCRT-I complex; ESCRT-I is |
| | | | | | | involved in ubiquitin-dependent sorting of proteins |
| | | | | | | into the endosome; suppressor of rna1-1 mutation; |
| | | | | | | may be involved in RNA export from nucleus |
| YLR164W | SHH4 | 1.63076 | 0.09 | 4.5 | SDHD | Mitochondrial inner membrane protein of unknown |
| | | | | | | function; similar to Tim18p; a fraction copurifies |
| | | | | | | with Sdh3p, but Shh4p is neither a stoichiometric |
| | | | | | | subunit of succinate dehydrogenase nor of the |
| | | | | | | TIM22 translocase; expression induced by nitrogen |
| | | | | | | limitation in a GLN3, GAT1-dependent manner; |
| | | | | | | SHH4 has a paralog, SDH4, that arose from the |
| | | | | | | whole genome duplication |
| YLR390W | ECM19 | 1.2306 | 0.1 | 4.5 | | Putative protein of unknown function; the authentic, |
| | | | | | | non-tagged protein is detected in highly purified |
| | | | | | | mitochondria in high-throughput studies |
| YMR322C | SNO4 | 2.03475 | 0.1 | 4.5 | PARK7 | Possible chaperone and cysteine protease; required |
| | | | | | | for transcriptional reprogramming during the |
| | | | | | | diauxic shift and for survival in stationary phase; |
| | | | | | | similar to bacterial Hsp31 and yeast Hsp31p, |
| | | | | | | Hsp32p, and Hsp33p; DJ-1/ThiJ/PfpI superfamily |
| | | | | | | member; predicted involvement in pyridoxine |
| | | | | | | metabolism; induced by mild heat stress and copper |
| | | | | | | deprivation |
| YNL007C | SIS1 | 1.15439 | 0.1 | 4.5 | DNAJ (B1-B9), | Type II HSP40 co-chaperone that interacts with the |
| | | | | | DNAJC5, | HSP70 protein Ssa1p; shuttles between cytosol and |
| | | | | | DNAJC5B, | nucleus; mediates delivery of misfolded proteins |
| | | | | | DNAJC5G | into the nucleus for degradation; involved in |
| | | | | | | proteasomal degradation of misfolded cytosolic |
| | | | | | | proteins; protein abundance increases in response to |
| | | | | | | DNA replication stress; polyQ aggregates sequester |
| | | | | | | Sis1p and interfere with clearance of misfolded |
| | | | | | | proteins; similar to bacterial DnaJ proteins and |
| | | | | | | mammalian DnaJB1 |
| YNL112W | DBP2 | −1.69614 | 0.1 | 4.5 | DDX17, | ATP-dependent RNA helicase of the DEAD-box |
| | | | | | DDX41, | protein family; has a strong preference for dsRNA; |
| | | | | | DDX43, | interacts with YRA1; required for the assembly of |
| | | | | | DDX5, | Yra1p, Nab2p and Mex67p onto mRNA and |
| | | | | | DDX53, | formation of nuclear mRNP; involved in mRNA |
| | | | | | DDX59 | decay and rRNA processing; may be involved in |
| | | | | | | suppression of transcription from cryptic initiation |
| | | | | | | sites |
| YOR054C | VHS3 | 1.07053 | 0.1 | 4.5 | PPCDC | Negative regulatory subunit of protein phosphatase |
| | | | | | | 1 Ppz1p; involved in coenzyme A biosynthesis; |
| | | | | | | subunit of the phosphopantothenoylcysteine |
| | | | | | | decarboxylase (PPCDC; Cab3p, Sis2p, Vhs3p) |
| | | | | | | complex and the CoA-Synthesizing Protein |
| | | | | | | Complex (CoA-SPC: Cab2p, Cab3p, Cab4p, Cab5p, |
| | | | | | | Sis2p and Vhs3p) |
| YPL280W | HSP32 | −9.59341 | 0.06 | 4.5 | PARK7 | Possible chaperone and cysteine protease; required |
| | | | | | | for transcriptional reprogramming during the |
| | | | | | | diauxic shift and for survival in stationary phase; |
| | | | | | | similar to E. coli Hsp31 and S. cerevisiae Hsp31p, |
| | | | | | | Hsp33p, and Sno4p; member of the DJ-1/ThiJ/PfpI |
| | | | | | | superfamily, which includes human DJ-1 involved |
| | | | | | | in Parkinson's disease and cancer |
| YDR358W | GGA1 | 1.24093 | 0.1 | 4.5 | GGA1 | Golgi-localized protein with homology to gamma- |
| | | | | | GGA2, | adaptin; interacts with and regulates Arf1p and |
| | | | | | GGA3, | Arf2p in a GTP-dependent manner in order to |
| | | | | | TOM1, | facilitate traffic through the late Golgi; GGA1 has a |
| | | | | | TOM1L1, | paralog, GGA2, that arose from the whole genome |
| | | | | | TOM1L2, | duplication |
| | | | | | WDFY1, | |
| | | | | | WDYF2 | |
| YEL020W-A | TIM9 | 3.84556 | 0.09 | 4.5 | TIMM9 | Essential protein of the mitochondrial |
| | | | | | | intermembrane space; forms a complex with |
| | | | | | | Tim10p (TIM10 complex) that delivers hydrophobic |
| | | | | | | proteins to the TIM22 complex for insertion into the |
| | | | | | | inner membrane |
| YDR171W | HSP42 | 1.43394 | 0.1 | 4 | CRYAA, | Small heat shock protein (sHSP) with chaperone |
| | | | | | CRYAB, | activity; forms barrel-shaped oligomers that |
| | | | | | HSPB1, | suppress unfolded protein aggregation; involved in |
| | | | | | HSPB3, | cytoskeleton reorganization after heat shock; protein |
| | | | | | HSPB6, | abundance increases and forms cytoplasmic foci in |
| | | | | | HSPB7, | response to DNA replication stress |
| | | | | | HSPB8, | |
| | | | | | HSPB9 | |
| YER121W | | 1.43396 | 0.1 | 4 | | Putative protein of unknown function; may be |
| | | | | | | involved in phosphatase regulation and/or |
| | | | | | | generation of precursor metabolites and energy |
| YGL258W-A | | 1.04446 | 0.1 | 4 | | Putative protein of unknown function |
| YGR247W | CPD1 | 1.06796 | 0.1 | 4 | | Cyclic nucleotide phosphodiesterase; hydrolyzes |
| | | | | | | ADP-ribose 1″, 2″-cyclic phosphate to ADP-ribose |
| | | | | | | 1″-phosphate; may have a role in tRNA splicing; no |
| | | | | | | detectable phenotype is conferred by null mutation |
| | | | | | | or by overexpression; protein abundance increases |
| | | | | | | in response to DNA replication stress |
| YLR149C | | 1.1335 | 0.1 | 4 | | Protein of unknown function; overexpression causes |
| | | | | | | a cell cycle delay or arrest; null mutation results in a |
| | | | | | | decrease in plasma membrane electron transport; |
| | | | | | | YLR149C is not an essential gene; protein |
| | | | | | | abundance increases in response to DNA replication |
| | | | | | | stress |
| YNL036W | NCE103 | 1.21279 | 0.1 | 4 | | Carbonic anhydrase; metalloenzyme that catalyzes |
| | | | | | | CO2 hydration to bicarbonate, which is an important |
| | | | | | | metabolic substrate, and protons; not expressed |
| | | | | | | under conditions of high CO2, such as inside a |
| | | | | | | growing colony, but transcription is induced in |
| | | | | | | response to low CO2 levels, such as on the colony |
| | | | | | | surface in ambient air; poorly transcribed under |
| | | | | | | aerobic conditions and at an undetectable level |
| | | | | | | under anaerobic conditions; abundance increases in |
| | | | | | | response to DNA replication stress |
| YOL114C | | 1.49153 | 0.1 | 4 | ICT1 | Putative protein of unknown function with similarity |
| | | | | | | to human ICT1; has prokaryotic factors that may |
| | | | | | | function in translation termination; YOL114C is not |
| | | | | | | an essential gene |
| YPL196W | OXR1 | 1.00292 | 0.1 | 4 | NCOA7, | Protein of unknown function required for oxidative |
| | | | | | OXR1, | damage resistance; required for normal levels of |
| | | | | | TLDC2 | resistance to oxidative damage; null mutants are |
| | | | | | | sensitive to hydrogen peroxide; member of a |
| | | | | | | conserved family of proteins found in eukaryotes |
| YBL039C | URA7 | −1.0346 | 0.1 | 3.5 | CTPS1, | Major CTP synthase isozyme (see also URA8); |
| | | | | | CTPS2 | catalyzes the ATP-dependent transfer of the amide |
| | | | | | | nitrogen from glutamine to UTP, forming CTP, the |
| | | | | | | final step in de novo biosynthesis of pyrimidines; |
| | | | | | | involved in phospholipid biosynthesis; capable of |
| | | | | | | forming cytoplasmic filaments termed |
| | | | | | | cytoophidium, especially during conditions of |
| | | | | | | glucose depletion; URA7 has a paralog, URA8, that |
| | | | | | | arose from the whole genome duplication |
| YDL199C | | 1.14338 | 0.09 | 3.5 | | Putative transporter; member of the sugar porter |
| | | | | | | family |
| YKL100C | | 1.01091 | 0.1 | 3.5 | HM13, | Putative protein of unknown function; has similarity |
| | | | | | SPPL2A, | to a human minor histocompatibility antigen and |
| | | | | | SPPL2C, | signal peptide peptidases; YKL100C is not an |
| | | | | | SPPL3 | essential gene |
| YMR244W | | −1.8109 | 0.1 | 3.5 | | Putative protein of unknown function |
| YNR002C | ATO2 | 1.08266 | 0.1 | 3.5 | | Putative transmembrane protein involved in export |
| | | | | | | of ammonia; ammonia is a starvation signal that |
| | | | | | | promotes cell death in aging colonies; |
| | | | | | | phosphorylated in mitochondria; member of the TC |
| | | | | | | 9.B.33 YaaH family; homolog of Y. lipolytica |
| | | | | | | Gpr1p; ATO2 has a paralog, ADY2, that arose from |
| | | | | | | the whole genome duplication |
| YOL084W | PHM7 | 1.58682 | 0.1 | 3.5 | TMEM63 (A-C) | Protein of unknown function; expression is |
| | | | | | | regulated by phosphate levels; green fluorescent |
| | | | | | | protein (GFP)-fusion protein localizes to the cell |
| | | | | | | periphery and vacuole; protein abundance increases |
| | | | | | | in response to DNA replication stress |
| YOR161C | PNS1 | 1.0314 | 0.1 | 3.5 | SLC44 (A1-A5) | Protein of unknown function; has similarity to |
| | | | | | | Torpedo californica tCTL1p, which is postulated to |
| | | | | | | be a choline transporter, neither null mutation nor |
| | | | | | | overexpression affects choline transport |
| YPL247C | | 1.49053 | 0.1 | 3.5 | DCAF7 | Putative protein of unknown function; green |
| | | | | | | fluorescent protein (GFP)-fusion protein localizes to |
| | | | | | | the cytoplasm and nucleus; similar to the petunia |
| | | | | | | WD repeat protein an11; overexpression causes a |
| | | | | | | cell cycle delay or arrest |
| YHR075C | PPE1 | 1.05839 | 0.09 | 3 | PPME1 | Protein with carboxyl methyl esterase activity; may |
| | | | | | | have a role in demethylation of the phosphoprotein |
| | | | | | | phosphatase catalytic subunit; also identified as a |
| | | | | | | small subunit mitochondrial ribosomal protein |
| YPL093W | NOG1 | −1.14212 | 0.1 | 3 | GTPBP4 | Putative GTPase; associates with free 60S ribosomal |
| | | | | | | subunits in the nucleolus and is required for 60S |
| | | | | | | ribosomal subunit biogenesis; constituent of 66S |
| | | | | | | pre-ribosomal particles; member of the ODN family |
| | | | | | | of nucleolar G-proteins |
| YFL012W | | 1.49125 | 0.1 | 2.5 | | Putative protein of unknown function; transcribed |
| | | | | | | during sporulation; null mutant exhibits increased |
| | | | | | | resistance to rapamycin |
| YGR128C | UTP8 | −1.00839 | 0.1 | 2.5 | | Nucleolar protein required for export of tRNAs |
| | | | | | | from the nucleus; also copurifies with the small |
| | | | | | | subunit (SSU) processome containing the U3 |
| | | | | | | snoRNA that is involved in processing of pre-18S |
| | | | | | | rRNA |
| YNL173C | MDG1 | 1.19461 | 0.1 | 2.5 | | Plasma membrane protein; involved in G-protein |
| | | | | | | mediated pheromone signaling pathway; |
| | | | | | | overproduction suppresses bem1 mutations; MDG1 |
| | | | | | | has a paralog, CRP1, that arose from the whole |
| | | | | | | genome duplication |
| YBR147W | RTC2 | 1.07812 | 0.1 | 2 | C3orf55, | Putative vacuolar membrane transporter for cationic |
| | | | | | PQLC2, | amino acids; likely contributes to amino acid |
| | | | | | TMEM44 | homeostasis by exporting cationic amino acids from |
| | | | | | | the vacuole; positive regulation by Lys14p suggests |
| | | | | | | that lysine may be the primary substrate; member of |
| | | | | | | the PQ-loop family, with seven transmembrane |
| | | | | | | domains; similar to mammalian PQLC2 vacuolar |
| | | | | | | transporter; RTC2 has a paralog, YPQ1, that arose |
| | | | | | | from the whole genome duplication |
| YCR098C | GIT1 | −1.01065 | 0.1 | 2 | | Plasma membrane permease; mediates uptake of |
| | | | | | | glycerophosphoinositol and glycerophosphocholine |
| | | | | | | as sources of the nutrients inositol and phosphate; |
| | | | | | | expression and transport rate are regulated by |
| | | | | | | phosphate and inositol availability |
| YDR074W | TPS2 | 1.10559 | 0.09 | 2 | | Phosphatase subunit of the trehalose-6-P |
| | | | | | | synthase/phosphatase complex; involved in |
| | | | | | | synthesis of the storage carbohydrate trehalose; |
| | | | | | | expression is induced by stress conditions and |
| | | | | | | repressed by the Ras-cAMP pathway; protein |
| | | | | | | abundance increases in response to DNA replication |
| | | | | | | stress |
| YDR345C | HXT3 | −1.5735 | 0.1 | 2 | | Low affinity glucose transporter of the major |
| | | | | | | facilitator superfamily; expression is induced in low |
| | | | | | | or high glucose conditions; HXT3 has a paralog, |
| | | | | | | HXT5, that arose from the whole genome |
| | | | | | | duplication |
| YGL101W | | 1.16132 | 0.1 | 2 | HDDC2 | Protein of unknown function; non-essential gene; |
| | | | | | | interacts with the DNA helicase Hpr5p; YGL101W |
| | | | | | | has a paralog, YBR242W, that arose from the whole |
| | | | | | | genome duplication |
| YHL021C | AIM17 | 1.00773 | 0.1 | 2 | BBOX1, | Putative protein of unknown function; the authentic, |
| | | | | | TMLHE | non-tagged protein is detected in highly purified |
| | | | | | | mitochondria in high-throughput studies; null |
| | | | | | | mutant displays reduced frequency of mitochondrial |
| | | | | | | genome loss |
| YIL101C | XBP1 | 1.02357 | 0.1 | 2 | | Transcriptional repressor; binds to promoter |
| | | | | | | sequences of the cyclin genes, CYS3, and SMF2; |
| | | | | | | expression is induced by stress or starvation during |
| | | | | | | mitosis, and late in meiosis; member of the |
| | | | | | | Swi4p/Mbp1p family; potential Cdc28p substrate; |
| | | | | | | relative distribution to the nucleus increases upon |
| | | | | | | DNA replication stress |
| YJL109C | UTP10 | −1.26035 | 0.1 | 2 | HEATR1 | Nucleolar protein; component of the small subunit |
| | | | | | | (SSU) processome containing the U3 snoRNA that |
| | | | | | | is involved in processing of pre-18S rRNA; mutant |
| | | | | | | has increased aneuploidy tolerance |
| YKR067W | GPT2 | 1.05353 | 0.1 | 2 | | Glycerol-3-phosphate/dihydroxyacetone phosphate |
| | | | | | | sn-1 acyltransferase; located in lipid particles and |
| | | | | | | the ER; involved in the stepwise acylation of |
| | | | | | | glycerol-3-phosphate and dihydroxyacetone in lipid |
| | | | | | | biosynthesis; the most conserved motifs and |
| | | | | | | functionally relevant residues are oriented towards |
| | | | | | | the ER lumen |
| YMR049C | ERB1 | −1.03363 | 0.1 | 2 | BOP1 | Constituent of 66S pre-ribosomal particles; forms a |
| | | | | | | complex with Nop7p and Ytm1p that is required for |
| | | | | | | maturation of the large ribosomal subunit; required |
| | | | | | | for maturation of the 25S and 5.8S ribosomal |
| | | | | | | RNAs; homologous to mammalian Bop1 |
| YMR290C | HAS1 | −1.23137 | 0.1 | 2 | DDX18 | ATP-dependent RNA helicase; involved in the |
| | | | | | | biogenesis of 40S and 60S ribosome subunits; |
| | | | | | | localizes to both the nuclear periphery and |
| | | | | | | nucleolus; highly enriched in nuclear pore complex |
| | | | | | | fractions; constituent of 66S pre-ribosomal particles |
| YNL305C | BXI1 | 1.07108 | 0.1 | 2 | FAIM2, | Protein involved in apoptosis; variously described as |
| | | | | | GRINA, | containing a BCL-2 homology (BH3) domain or as |
| | | | | | TMBIM1, | a member of the BAX inhibitor family; reported to |
| | | | | | TMBIM4 | promote apoptosis under some conditions and to |
| | | | | | | inhibit it in others; localizes to ER and vacuole; may |
| | | | | | | link the unfolded protein response to apoptosis via |
| | | | | | | regulation of calcium-mediated signaling; |
| | | | | | | translocates to mitochondria under apoptosis- |
| | | | | | | inducing conditions in a process involving Mir1p |
| | | | | | | and Cor1p |
| YPL230W | USV1 | 1.19881 | 0.1 | 2 | KLF (1-17), | Putative transcription factor containing a C2H2 zinc |
| | | | | | SP (5-7) | finger; mutation affects transcriptional regulation of |
| | | | | | | genes involved in growth on non-fermentable |
| | | | | | | carbon sources, response to salt stress and cell wall |
| | | | | | | biosynthesis; USV1 has a paralog, RGM1, that |
| | | | | | | arose from the whole genome duplication |
| YGR230W | BNS1 | 1.08088 | 0.09 | 2 | | Protein of unknown function; overexpression |
| | | | | | | bypasses need for Spo12p, but not required for |
| | | | | | | meiosis; BNS1 has a paralog, SPO12, that arose |
| | | | | | | from the whole genome duplication |
| YPL123C* | RNY1 | 1.3355 | 0.1 | 2 | RNASET2 | Vacuolar RNase of the T(2) family; relocalizes to |
| | | | | | | the cytosol where it cleaves tRNAs upon oxidative |
| | | | | | | or stationary phase stress; promotes apoptosis under |
| | | | | | | stress conditions and this function is independent of |
| | | | | | | its catalytic activity |
| YBR126W-A | | 1.27059 | 0.1 | 1 | | Protein of unknown function; identified by gene- |
| | | | | | | trapping, microarray analysis, and genome-wide |
| | | | | | | homology searches; mRNA identified as translated |
| | | | | | | by ribosome profiling data; partially overlaps the |
| | | | | | | dubious ORF YBR126W-B |
| YCL073C | GEX1 | 5.11103 | 0.1 | 1 | | Proton:glutathione antiporter; localized to the |
| | | | | | | vacuolar and plasma membranes; imports |
| | | | | | | glutathione from the vacuole and exports it through |
| | | | | | | the plasma membrane; has a role in resistance to |
| | | | | | | oxidative stress and modulation of the PKA |
| | | | | | | pathway; GEX1 has a paralog, GEX2, that arose |
| | | | | | | from a segmental duplication |
| YCR021C* | HSP30 | 1.3464 | 0.1 | 1 | | Negative regulator of the H(+)-ATPase Pma1p; |
| | | | | | | stress-responsive protein; hydrophobic plasma |
| | | | | | | membrane localized; induced by heat shock, ethanol |
| | | | | | | treatment, weak organic acid, glucose limitation, |
| | | | | | | and entry into stationary phase |
| YDL110C | TMA17 | 1.27099 | 0.1 | 1 | | Protein of unknown function that associates with |
| | | | | | | ribosomes; heterozygous deletion demonstrated |
| | | | | | | increases in chromosome instability in a rad9 |
| | | | | | | deletion background; protein abundance is |
| | | | | | | decreased upon intracellular iron depletion |
| YDR516C | EMI2 | 1.40186 | 0.1 | 1 | GCK, HK1, | Non-essential protein of unknown function; required |
| | | | | | HK2, HK3, | for transcriptional induction of the early meiotic- |
| | | | | | HKDC1 | specific transcription factor IME1; required for |
| | | | | | | sporulation; expression regulated by glucose- |
| | | | | | | repression transcription factors Mig1/2p; EMI2 has |
| | | | | | | a paralog, GLK1, that arose from the whole genome |
| | | | | | | duplication; protein abundance increases in response |
| | | | | | | to DNA replication stress |
| YEL012W* | UBC8 | 1.07261 | 0.1 | 1 | UBE2H | Ubiquitin-conjugating enzyme that regulates |
| | | | | | | gluconeogenesis; negatively regulates |
| | | | | | | gluconeogenesis by mediating the glucose-induced |
| | | | | | | ubiquitination of fructose-1,6-bisphosphatase |
| | | | | | | (FBPase); cytoplasmic enzyme that catalyzes the |
| | | | | | | ubiquitination of histones in vitro |
| YER053C-A | | 1.01554 | 0.1 | 1 | | Protein of unknown function; green fluorescent |
| | | | | | | protein (GFP)-fusion protein localizes to the |
| | | | | | | endoplasmic reticulum; protein abundance increases |
| | | | | | | in response to DNA replication stress |
| YFR042W | KEG1 | 1.02711 | 0.1 | 1 | | Integral membrane protein of the ER; physically |
| | | | | | | interacts with Kre6p; has a role in the synthesis of |
| | | | | | | beta-1,6-glucan in the cell wall; required for cell |
| | | | | | | viability |
| YGR130C* | | 1.177 | 0.1 | 1 | | Component of the eisosome with unknown function; |
| | | | | | | GFP-fusion protein localizes to the cytoplasm; |
| | | | | | | specifically phosphorylated in vitro by mammalian |
| | | | | | | diphosphoinositol pentakisphosphate (IP7) |
| YGR131W | FHN1 | 1.11873 | 0.1 | 1 | | Protein of unknown function; induced by |
| | | | | | | ketoconazole; promoter region contains sterol |
| | | | | | | regulatory element motif, which has been identified |
| | | | | | | as a Upc2p-binding site; overexpression |
| | | | | | | complements function of Nce102p in NCE102 |
| | | | | | | deletion strain; FHN1 has a paralog, NCE102, that |
| | | | | | | arose from the whole genome duplication |
| YHR171W | ATG7 | 1.26014 | 0.1 | 1 | ATG7 | Autophagy-related protein and dual specificity |
| | | | | | | member of the E1 family; mediates the conjugation |
| | | | | | | of Atg12p with Atg5p and Atg8p with |
| | | | | | | phosphatidylethanolamine which are required steps |
| | | | | | | in autophagosome formation; E1 enzymes are also |
| | | | | | | known as ubiquitin-activating enzymes; involved in |
| | | | | | | methionine restriction extension of chronological |
| | | | | | | lifespan in an autophagy-dependent manner |
| YHR197W | RIX1 | −1.00962 | 0.1 | 1 | | Component of the Rix1 complex and possibly pre- |
| | | | | | | replicative complexes; required for processing of |
| | | | | | | ITS2 sequences from 35S pre-rRNA; component of |
| | | | | | | the pre-60S ribosomal particle with the dynein- |
| | | | | | | related AAA-type ATPase Mdn1p; required for pre- |
| | | | | | | replicative complex (pre-RC) formation and |
| | | | | | | maintenance during DNA replication licensing; |
| | | | | | | relocalizes to the cytosol in response to hypoxia; |
| | | | | | | essential gene |
| YJL161W | FMP33 | 1.16756 | 0.1 | 1 | | Putative protein of unknown function; the authentic, |
| | | | | | | non-tagged protein is detected in highly purified |
| | | | | | | mitochondria in high-throughput studies |
| YJL163C | | 1.51658 | 0.1 | 1 | SLC46 (A1-A3) | Putative protein of unknown function |
| YKL221W | MCH2 | 1.07998 | 0.09 | 1 | SLC16 (A1-A14) | Protein with similarity to mammalian |
| | | | | | | monocarboxylate permeases; monocarboxylate |
| | | | | | | permeases are involved in transport of |
| | | | | | | monocarboxylic acids across the plasma membrane |
| | | | | | | but mutant is not deficient in monocarboxylate |
| | | | | | | transport |
| YLR257W | | 1.00554 | 0.1 | 1 | | Protein of unknown function; protein abundance |
| | | | | | | increases in response to DNA replication stress |
| YML052W | SUR7 | 1.00544 | 0.1 | 1 | | Plasma membrane protein of unknown function |
| | | | | | | involved with endocytosis; associated with |
| | | | | | | endocytosis along with Pil1p and Lsp1p; component |
| | | | | | | of eisosomes; sporulation and plasma membrane |
| | | | | | | sphingolipid content are altered in mutants; localizes |
| | | | | | | to furrow-like invaginations (MCC patches) |
| YMR128W | ECM16 | −1.03422 | 0.1 | 1 | DHX37 | Essential DEAH-box ATP-dependent RNA helicase |
| | | | | | | specific to U3 snoRNP; predominantly nucleolar in |
| | | | | | | distribution; required for 18S rRNA synthesis |
| YNL141W | AAH1 | −1.67229 | 0.1 | 1 | ADA, ADAL | Adenine deaminase (adenine aminohydrolase); |
| | | | | | | converts adenine to hypoxanthine; involved in |
| | | | | | | purine salvage; transcriptionally regulated by |
| | | | | | | nutrient levels and growth phase; Aah1p degraded |
| | | | | | | upon entry into quiescence via SCF and the |
| | | | | | | proteasome |
| YOL032W | OPI10 | 1.67029 | 0.1 | 1 | C11orf73 | Protein with a possible role in phospholipid |
| | | | | | | biosynthesis; null mutant displays an inositol- |
| | | | | | | excreting phenotype that is suppressed by |
| | | | | | | exogenous choline; protein abundance increases in |
| | | | | | | response to DNA replication stress |
| YOL048C | RRT8 | 1.17182 | 0.1 | 1 | | Protein involved in spore wall assembly; shares |
| | | | | | | similarity with Lds1p and Lds2p and a strain mutant |
| | | | | | | for all 3 genes exhibits reduced dityrosine |
| | | | | | | fluorescence relative to the single mutants; |
| | | | | | | identified in a screen for mutants with increased |
| | | | | | | levels of rDNA transcription; green fluorescent |
| | | | | | | protein (GFP)-fusion protein localizes to lipid |
| | | | | | | particles; protein abundance increases in response to |
| | | | | | | DNA replication stress |
| YOR280C | FSH3 | 1.01108 | 0.1 | 1 | OVCA2 | Putative serine hydrolase; likely target of Cyc8p- |
| | | | | | | Tup1p-Rfx1p transcriptional regulation; sequence is |
| | | | | | | similar to S. cerevisiae Fsh1p and Fsh2p and the |
| | | | | | | human candidate tumor suppressor OVCA2 |
| YPL012W | RRP12 | −1.25061 | 0.1 | 1 | RRP12 | Protein required for export of the ribosomal |
| | | | | | | subunits; associates with the RNA components of |
| | | | | | | the pre-ribosomes; has a role in nuclear import in |
| | | | | | | association with Pse1p; also plays a role in the cell |
| | | | | | | cycle and the DNA damage response; contains |
| | | | | | | HEAT-repeats |
| YPL226W | NEW1 | −1.07183 | 0.1 | 1 | | ATP binding cassette protein; cosediments with |
| | | | | | | polysomes and is required for biogenesis of the |
| | | | | | | small ribosomal subunit; Asn/Gln-rich rich region |
| | | | | | | supports [NU+] prion formation and susceptibility |
| | | | | | | to [PSI+] prion induction |
| YBR238C | | −1.27286 | 0.1 | 0 | | Mitochondrial membrane protein; not required for |
| | | | | | | respiratory growth but causes a synthetic respiratory |
| | | | | | | defect in combination with rmd9 mutations; |
| | | | | | | transcriptionally up-regulated by TOR; deletion |
| | | | | | | increases life span; YBR238C has a paralog, |
| | | | | | | RMD9, that arose from the whole genome |
| | | | | | | duplication |
| YBR296C | PHO89 | −2.36723 | 0.1 | 0 | SLC20A1, | Plasma membrane Na+/Pi cotransporter; active in |
| | | | | | SLC20A2 | early growth phase; similar to phosphate |
| | | | | | | transporters of Neurospora crassa; transcription |
| | | | | | | regulated by inorganic phosphate concentrations and |
| | | | | | | Pho4p; mutations in related human transporter genes |
| | | | | | | hPit1 and hPit2 are associated with |
| | | | | | | hyperphosphatemia-induced calcification of |
| | | | | | | vascular tissue and familial idiopathic basal ganglia |
| | | | | | | calcification |
| YDL018C | ERP3 | 1.21249 | 0.1 | 0 | TMED1, | Protein with similarity to Emp24p and Erv25p; |
| | | | | | TMED2, | member of the p24 family involved in ER to Golgi |
| | | | | | TMED3, | transport |
| | | | | | TMED4, | |
| | | | | | TMED5, | |
| | | | | | TMED6, | |
| | | | | | TMED7, | |
| | | | | | TMED- | |
| | | | | | TICAM2 | |
| YDR100W | TVP15 | 1.38301 | 0.1 | 0 | | Integral membrane protein; localized to late Golgi |
| | | | | | | vesicles along with the v-SNARE Tlg2p |
| YEL039C | CYC7 | 1.2486 | 0.1 | 0 | CYC5 | Cytochrome c isoform 2, expressed under hypoxic |
| | | | | | | conditions; electron carrier of the mitochondrial |
| | | | | | | intermembrane space that transfers electrons from |
| | | | | | | ubiquinone-cytochrome c oxidoreductase to |
| | | | | | | cytochrome c oxidase during cellular respiration; |
| | | | | | | protein abundance increases in response to DNA |
| | | | | | | replication stress; CYC7 has a paralog, CYC1, that |
| | | | | | | arose from the whole genome duplication |
| YER054C | GIP2 | 1.13455 | 0.1 | 0 | PPP1R3 (A-G) | Putative regulatory subunit of protein phosphatase |
| | | | | | | Glc7p; involved in glycogen metabolism; contains a |
| | | | | | | conserved motif (GVNK motif) that is also found in |
| | | | | | | Gac1p, Pig1p, and Pig2p; GIP2 has a paralog, PIG2, |
| | | | | | | that arose from the whole genome duplication |
| YFR003C | YPI1 | 1.05663 | 0.1 | 0 | PPP1R11 | Regulatory subunit of the type I protein phosphatase |
| | | | | | | (PP1) Glc7p; Glc7p participates in the regulation of |
| | | | | | | a variety of metabolic processes including mitosis |
| | | | | | | and glycogen metabolism; in vitro evidence |
| | | | | | | suggests Ypi1p is an inhibitor of Glc7p while in |
| | | | | | | vivo evidence suggests it is an activator; |
| | | | | | | overproduction causes decreased cellular content of |
| | | | | | | glycogen; partial depletion causes lithium |
| | | | | | | sensitivity, while overproduction confers lithium- |
| | | | | | | tolerance |
| YGL120C | PRP43 | −1.12706 | 0.1 | 0 | DHX15, | RNA helicase in the DEAH-box family; functions in |
| | | | | | DHX32, | both RNA polymerase I and polymerase II transcript |
| | | | | | DQX1 | metabolism; catalyzes removal of U2, U5, and U6 |
| | | | | | | snRNPs from the postsplicing lariat-intron |
| | | | | | | ribonucleoprotein complex; required for efficient |
| | | | | | | biogenesis of both small- and large-subunit rRNAs; |
| | | | | | | acts with Sqs1p to promote 20S to 18S rRNA |
| | | | | | | processing catalyzed by endonuclease Nob1p |
| YHR126C | ANS1 | −1.85683 | 0.1 | 0 | | Putative GPI protein; transcription dependent upon |
| | | | | | | Azf1p |
| YIL053W | GPP1 | −1.13681 | 0.1 | 0 | | Constitutively expressed DL-glycerol-3-phosphate |
| | | | | | | phosphatase; also known as glycerol-1-phosphatase; |
| | | | | | | involved in glycerol biosynthesis, induced in |
| | | | | | | response to both anaerobic and osmotic stress; |
| | | | | | | GPP1 has a paralog, GPP2, that arose from the |
| | | | | | | whole genome duplication |
| YLL052C | AQY2 | −1.6217 | 0.1 | 0 | AQP(1-10), | Water channel that mediates water transport across |
| | | | | | MIP | cell membranes; only expressed in proliferating |
| | | | | | | cells; controlled by osmotic signals; may be |
| | | | | | | involved in freeze tolerance; disrupted by a stop |
| | | | | | | codon in many S. cerevisiae strains |
| YML123C | PHO84 | −2.08859 | 0.09 | 0 | | High-affinity inorganic phosphate (Pi) transporter; |
| | | | | | | also low-affinity manganese transporter; regulated |
| | | | | | | by Pho4p and Spt7p; mutation confers resistance to |
| | | | | | | arsenate; exit from the ER during maturation |
| | | | | | | requires Pho86p; cells overexpressing Pho84p |
| | | | | | | accumulate heavy metals but do not develop |
| | | | | | | symptoms of metal toxicity |
| YOR292C | | 1.25053 | 0.1 | 0 | MPV17 | Putative protein of unknown function; green |
| | | | | | | fluorescent protein (GFP)-fusion protein localizes to |
| | | | | | | the vacuole; YOR292C is not an essential gene |
| YPR151C | SUE1 | 1.05337 | 0.1 | 0 | | Protein required for degradation of unstable forms |
| | | | | | | of cytochrome c; located in the mitochondria |
| YAL028W | FRT2 | 1.09066 | 0.09 | N/A | | Tail-anchored ER membrane protein of unknown |
| | | | | | | function; interacts with homolog Frt1p; promotes |
| | | | | | | growth in conditions of high Na+, alkaline pH, or |
| | | | | | | cell wall stress, possibly via a role in |
| | | | | | | posttranslational translocation; potential Cdc28p |
| | | | | | | substrate; FRT2 has a paralog, FRT1, that arose |
| | | | | | | from the whole genome duplication |
| YBR230W-A | | 1.1175 | 0.1 | N/A | | Putative protein of unknown function; YBR230W-A |
| | | | | | | has a paralog, COQ8, that arose from the whole |
| | | | | | | genome duplication |
| YBR285W | | 1.34955 | 0.1 | N/A | | Putative protein of unknown function; YBR285W is |
| | | | | | | not an essential gene |
| YBR302C | COS2 | 1.10159 | 0.1 | N/A | | Protein of unknown function; member of the |
| | | | | | | DUP380 subfamily of conserved, often |
| | | | | | | subtelomerically-encoded proteins |
| YDR169C-A | | 8.81987 | 0.09 | N/A | | Putative protein of unknown function; identified by |
| | | | | | | fungal homology and RT-PCR |
| YDR258C | HSP78 | 1.305 | 0.1 | N/A | CLPB | Oligomeric mitochondrial matrix chaperone; |
| | | | | | | cooperates with Ssc1p in mitochondrial |
| | | | | | | thermotolerance after heat shock; able to prevent the |
| | | | | | | aggregation of misfolded proteins as well as |
| | | | | | | resolubilize protein aggregates |
| YDR342C | HXT7 | 1.10241 | 0.09 | N/A | | High-affinity glucose transporter; member of the |
| | | | | | | major facilitator superfamily, nearly identical to |
| | | | | | | Hxt6p, expressed at high basal levels relative to |
| | | | | | | other HXTs, expression repressed by high glucose |
| | | | | | | levels; HXT7 has a paralog, HXT4, that arose from |
| | | | | | | the whole genome duplication |
| YGR027W-B | | −6.3627 | 0.07 | N/A | | Retrotransposon TYA Gag and TYB Pol genes; |
| | | | | | | transcribed/translated as one unit; polyprotein is |
| | | | | | | processed to make a nucleocapsid-like protein |
| | | | | | | (Gag), reverse transcriptase (RT), protease (PR), |
| | | | | | | and integrase (IN); similar to retroviral genes |
| YHR086W-A | | 1.75662 | 0.1 | N/A | | Putative protein of unknown function; identified by |
| | | | | | | fungal homology and RT-PCR |
| YHR087W | RTC3 | 1.27268 | 0.1 | N/A | | Protein of unknown function involved in RNA |
| | | | | | | metabolism; has structural similarity to SBDS, the |
| | | | | | | human protein mutated in Shwachman-Diamond |
| | | | | | | Syndrome (the yeast SBDS ortholog = SDO1); null |
| | | | | | | mutation suppresses cdc13-1 temperature |
| | | | | | | sensitivity; protein abundance increases in response |
| | | | | | | to DNA replication stress |
| YJR005C-A | | 1.37524 | 0.1 | N/A | CCDC124 | Putative protein of unknown function; originally |
| | | | | | | identified as a syntenic homolog of an <i>Ashbya |
| | | | | | | gossypii</i> gene; YJR005C-A has a paralog, |
| | | | | | | YGR169C-A, that arose from the whole genome |
| | | | | | | duplication |
| YLR401C | DUS3 | −1.02504 | 0.07 | N/A | DUS3L | Dihydrouridine synthase; member of a widespread |
| | | | | | | family of conserved proteins including Smm1p, |
| | | | | | | Dus1p, and Dus4p; contains a consensus oleate |
| | | | | | | response element (ORE) in its promoter region; |
| | | | | | | forms nuclear foci upon DNA replication stress |
| YML132W | COS3 | 1.10159 | 0.1 | N/A | | Protein involved in salt resistance; interacts with |
| | | | | | | sodium:hydrogen antiporter Nha1p; member of the |
| | | | | | | DUP380 subfamily of conserved, often |
| | | | | | | subtelomerically-encoded proteins |
| YMR247W-A | | 1.41759 | 0.1 | N/A | | Putative protein of unknown function |
| YMR262W | | 1.37104 | 0.1 | N/A | TATDN3 | Protein of unknown function; interacts weakly with |
| | | | | | | Knr4p; YMR262W is not an essential gene |
| YOL161C | PAU20 | 9.02934 | 0.09 | N/A | | Protein of unknown function; member of the |
| | | | | | | seripauperin multigene family encoded mainly in |
| | | | | | | subtelomeric regions; expression induced by low |
| | | | | | | temperature and also by anaerobic conditions; |
| | | | | | | induced during alcoholic fermentation |
| YOL164W-A | | 1.0321 | 0.09 | N/A | | Putative protein of unknown function; identified by |
| | | | | | | fungal homology and RT-PCR |
| YOR341W | RPA190 | −1.11917 | 0.1 | N/A | POLR1A | RNA polymerase I largest subunit A190 |
| YPR010C | RPA135 | −1.28417 | 0.1 | N/A | POLR1B | RNA polymerase I second largest subunit A135 |

Neurodegenerative Disorders Associated with α-Synuclein Dysfunction

Aspects of the disclosure provide methods for treating neurodegenerative disorders associated with α-synuclein dysfunction by administering an agent that modulates expression and/or activity of a human homolog of any of the genes set forth in Table 1. The term “neurodegenerative disorders” encompasses many disorders that are characterized by progressive nervous system dysfunction and/or death of neurons and may include both hereditary and sporadic disorders. Neurodegenerative disorders may affect a subject's movement, sensory function, and/or mental function, such as memory.

A subset of neurodegenerative disorders is associated with α-synuclein dysfunction. As used herein, a neurodegenerative disorder is “associated” with α-synuclein dysfunction, if the disorder involves or is characterized by α-synuclein dysfunction, such as α-synuclein aggregation. A neurodegenerative disorder associated with α-synuclein dysfunction may also be referred to as a synucleinopathy.

Alpha-synuclein, also used interchangeably with α-synuclein or αSyn, is an abundant protein found in the brain. Alpha-synuclein is encoded by the gene SCNA (also referred to as NACP, PARK1, PARK4, or PD1) and may be present in any of three distinct isoforms generated due to alternative splicing of the α-synuclein-encoding transcript. Under normal conditions, α-synuclein is thought to be important in synaptic activity, neuronal golgi function, and/or vesicle trafficking and essential for normal cognitive function. Although the specific function of α-synuclein has not been determined, it is generally present as a soluble cytoplasmic protein that is capable of binding cellular membranes. Snead et al. Experimental Neurology (2014) 23(4): 292-313.

As used herein, the term “α-synuclein dysfunction” refers to α-synuclein in an altered state, thereby disrupting any of the functions in which α-synuclein may be involved. In some embodiments, dysfunctional α-synuclein may have a reduced function (activity) or the α-synuclein may be non-functional. For example, in some instances, α-synuclein may be misfolded and form aggregates of insoluble fibrils within a cell (e.g., a neural cell), referred to as Lewy bodies or Lewy neurites. The insoluble α-synuclein aggregates are deposited and accumulate in neurons, nerve fibers, and/or glial cells. Lewy bodies and Lewy neurites may include additional proteins, such as ubiquitin. The presence of Lewy bodies and/or Lew neurites may be visualized by microscopy and is considered a pathological hallmark of disorders associated with α-synuclein dysfunction, such as Parkinson's disease. Disorders associated with α-synuclein dysfunction may be also be referred to as synucleinopathies.

Examples of neurodegenerative disorders associated with α-synuclein dysfunction include, without limitation. Parkinson's disease (PD). Lewy body variant of Alzheimer's disease, diffuse Lewy body disease, dementia with Lewy bodies, multiple system atrophy, and neurodegeneration with brain iron accumulation type 1.

Aspects of the present disclosure provide methods of treating a neurodegenerative disorder associated with α-synuclein dysfunction by administering an agent to a subject having the disorder associated with α-synuclein dysfunction. In some embodiments, the subject is assessed to determine whether the subject has a disorder associated with α-synuclein dysfunction or to determine the severity of the disorder associated with α-synuclein dysfunction prior to administering the one or more agent. Methods for diagnosing a disorder, determining whether a subject may be at risk of developing a disorder, or assessing the severity of disorders associated with α-synuclein dysfunction are known in the art and may include, for example, sequencing or analyzing the SCNA loci for multiplications of the α-synuclein-encoding gene and/or mutations (e.g., single nucleotide polymorphisms) in the SCNA open reading frame; evaluating the subject's family history; evaluating the subject's neurological history; and/or performing a neurological examination, which may include evaluation of the subject's physical movement. Symptoms vary between subject but may include motor symptoms, such as shaking or tremor, slowness of movement (bradykinesia); stiffness in the arms, legs, or trunk; problems with balance.

In some embodiments of the methods described herein, the neurodegenerative disorder associated with α-synuclein dysfunction is Parkinson's disease. The incidence of Parkinson's Disease has been associated with misfiling and/or loss of function of α-synuclein. In general, Parkinson's Disease may be classified as familial (hereditary) Parkinson's Disease or idiopathic (sporadic) Parkinson's Disease. Familial Parkinson's disease has been associated with mutations in the SNCA gene encoding α-synuclein, for example the single nucleotide polymorphisms (snp) A53T, A30P, E46K, H50Q, and G51D. Mutant forms of α-synuclein have been found to form insoluble fibrils more rapidly and may have an increase propensity to aggregate as compared to wild-type α-synuclein. In some instances, familial Parkinson's disease has been associated with duplication or triplication of the SNCA locus.

Although there are currently no curative therapies for Parkinson's disease, conventional therapies aim to treat (ameliorate) the symptoms associated with Parkinson's disease.

### Agents

The methods described herein involve administering a therapeutically effective amount of an agent that enhances expression and/or activity of a human homolog of one or more genes set forth in Table 1. An agent that enhances the expression and/or activity of a human homolog of one or more genes set forth in Table 1 may be administered according to any of the methods described herein. An agent may selectively enhance expression and/or activity of one or a small number of related genes (e.g., genes encoding proteins with related functions, structures, or belonging to the same protein family).

In general, expression of a gene (e.g., a nucleic acid that may encode a protein) can be enhanced by any of a variety of methods, for example by modulating transcription, mRNA localization, mRNA degradation, mRNA stability, and/or translation of the protein. In some embodiments, the agent enhances expression of a gene by promoting or inhibiting transcription of the nucleic acid. In other embodiments, the agent enhances expression of a nucleic acid by promoting or inhibiting mRNA localization, mRNA degradation or mRNA stability. In other embodiments, the agent enhances expression of a nucleic acid by promoting or inhibiting translation of the nucleic acid. In other embodiments, an agent enhances protein levels by modulating protein stability or protein degradation.

In some embodiments, the agent enhances expression of human homolog of at least one gene provided in Table 1 such that the amount of the protein or the amount of a nucleic acid is enhanced relative to the amount of the protein or the amount of the nucleic acid in the absence of the agent. In some embodiments, the amount of the protein or the amount of a nucleic acid is enhanced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the amount of the protein or the amount of the nucleic acid in the absence of the agent. In some embodiments, the amount of the protein or the amount of a nucleic acid in the presence of the agent is about 5%, 10%, 15%, 20%, 25%, 30%, 35%, 40%, 45%, 50%, 55%, 60%, 65%, 70%, 75%, 80%, 85%, 90%, or about 95% more than the amount of protein or nucleic acid that is produced in the absence of the agent.

The agent can enhance the activity of a human homolog of one or more genes provided in Table 1 with or without modulating the nucleic acid, for example by enhancing the activity of a protein encoded by the gene. In some embodiments, the agent interacts with the protein directly or indirectly, thereby enhancing the activity of the protein. In some embodiments, the agent may enhance the activity of a protein by modulating protein stability, protein degradation, one or more protein interactions, enzymatic activity, conformation, and or signaling activity. In other embodiments, an agent renders a protein constitutively active.

In some embodiments, the agent enhances activity of the protein such that the activity of the protein is enhanced relative to the activity of the protein in the absence of the agent. In some embodiments, the activity of the protein is enhanced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the activity of the protein in the absence of the agent.

Methods for assessing the expression and/or activity of a gene or gene product (e.g., a protein) will be evident to one of ordinary skill in the art and can be conducted in vitro or in vivo. Methods may involve collecting one or more biological samples from a subject. In some embodiments, expression and/or activity of the gene or gene product is assessed prior to and/or after administration of the agent to the subject. Methods can involve measuring the level of mRNA and/or protein, and/or measuring the activity of a gene product, such as an enzymatic activity or signaling activity.

An agent that enhances the expression and/or activity of a human homolog of one or more genes provided in Table 1 may be in any form known in the art. For example, in some embodiments, the agent is a small molecule, a protein, or a nucleic acid. In some embodiments, more than one agent is administered to the subject (e.g., 1, 2, 3, 4, 5, or more) agents. In some embodiments, more than one agent is administered to the subject, each of which enhances the expression and/or activity of a human homolog of a different gene provided by Table 1. In some embodiments, more than one agent is administered to the subject, each of which enhances the expression and/or activity of a human homolog of the same gene provided by Table 1.

In some embodiments, the agent is a protein that enhances the expression and/or activity of a human homolog of one or more genes presented in Table 1. In some embodiments, the protein is a recombinant protein. In some embodiments, the protein or fusion protein enhances the expression of the protein by enhancing transcription of the gene, for example by interacting with one or more components involved in the transcription process. In some embodiments, the protein or fusion protein enhances the expression of the protein by reducing degradation of a transcript of the gene. In some embodiments, the protein enhances the activity of a protein (encoded by the gene), for example by interacting with the protein directly or indirectly.

In some embodiments, the protein is a protein encoded by a human homolog of a gene provided in Table 1. In general, administering a protein that is a protein encoded by a human homolog of a gene provided in Table 1 may enhance the activity of such a protein by increasing the abundance of the protein in the subject or in a cell. Also within the scope of the present invention are modified proteins, such as proteins encoding one or more mutations relative to the wild-type protein. In some embodiments, a protein maybe modified to modulate activity of the protein. In some embodiments, the modified proteins are proteins encoded by a human homolog of a gene provided in Table 1, wherein the protein has been modified (e.g., mutated) to have enhanced activity.

In some embodiments, the agent is a small molecule that enhances the expression and/or activity of a human homolog of one or more genes presented in Table 1. As used herein, a “small molecule,” including small molecule inhibitors and small molecule activators, refers to a compound having a low molecular weight (i.e., less than 900 Daltons). In some embodiments, the small molecule enhances expression and/or activity of a human homolog of a gene presented in Table 1. In some embodiments, the small molecule modulates expression of the protein by inhibiting or preventing transcription or translation of an inhibitor that prevents or reduces expression and/or activity of the targeted gene. In some embodiments, the small molecule enhances the expression of the targeted gene by promoting the transcription or translation of the gene, e.g., by interacting with a component of the transcription or translation machinery. In some embodiments, the small molecule enhances the activity of the target gene by promoting the activity of the gene product encoded by the targeted gene. For example, the small molecule may interact with a protein encoded by the gene and maintain the protein in an active conformation. In some embodiments, the small molecule enhances the activity of a protein, for example by interacting with the protein encoded by the target gene directly or indirectly. In one example, the small molecule is sulforaphane, an inducer of TXN.

In some embodiments, the agent is a nucleic acid that enhances the expression and/or activity of a human homolog of at least one gene presented in Table 1. In some embodiments, the nucleic acid enhances expression of the targeted gene(s) by inhibiting or preventing transcription of a nucleic acid encoding an inhibitor of the targeted gene or the gene product encoded thereby. In some embodiments, the nucleic acid enhances expression of the targeted gene(s) by inhibiting or preventing translation of an inhibitor of the gene or gene product and/or by modulating mRNA degradation. In some embodiments, the nucleic acid modulates the activity of a gene product encoded by the gene, for example through protein-nucleic acid interactions. Examples of nucleic acids that may enhance the expression and/or activity of a human homolog of one or more genes presented in Table 1 include, without limitation, CRISPR/Cas guideRNAs (gRNAs), siRNAs, miRNA, shRNAs, and nucleic acids (DNA or RNA) encoding a protein, such as a protein encoded by a human homolog of any of the genes provided in Table 1.

In some embodiments, the agent is a CRISPR/Cas guide RNA (gRNA). The terms “gRNA,” “guide RNA” and “CRISPR guide sequence” may be used interchangeably throughout and refer to a nucleic acid comprising a sequence that determines the specificity of a Cas DNA binding protein (or variant thereof) of a CRISPR/Cas system. A gRNA has a level of complementary to one or more nucleic acid sequences in a cell that is sufficient for the gRNA to hybridize to the nucleic acid sequence. The gRNA or portion thereof that is complementary to the a nucleic acid sequence may be between 15-25 nucleotides, 18-22 nucleotides, or 19-21 nucleotides in length. In some embodiments, the gRNA sequence that is complementary to the target nucleic acid is 15, 16, 17, 18, 19, 20, 21, 22, 23, 24, or 25 nucleotides in length. In some embodiments, the gRNA sequence that is complementary to the target nucleic acid is 20 nucleotides in length.

In some embodiments, the gRNA has one or more mismatches relative to the nucleic acid sequence but retains sufficient complementarity such that the gRNA is capable of hybridizing to a target nucleic acid sequence. In some embodiments, one or more (e.g., 2, 3, 4, 5, or more) mismatches may be incorporated into the gRNA, or into a portion of the gRNA, such that the gRNA may hybridize at multiple genetic loci in the cell. In some embodiments, the gRNA is capable of hybridizing to multiple, non-identical target nucleic acid sequences in the cell. In some embodiments, the target nucleic acid sequence is present at multiple genetic loci in the cell.

In addition to a sequence that is sufficiently complementary a target nucleic acid, in some embodiments, the gRNA also comprises a scaffold sequence. Expression of a gRNA encoding both a sequence with complementarity to a target nucleic acid and scaffold sequence has the dual function of both binding (hybridizing) to the target nucleic acid and recruiting a Cas protein (or variant thereof) to the target nucleic acid, which may result in site-specific CRISPR activity. In some embodiments, such a chimeric gRNA may be referred to as a single guide RNA (sgRNA).

As used herein, a “scaffold sequence,” also referred to as a tracrRNA, refers to a nucleic acid sequence that recruits a CRISPR protein (or variant thereof, e.g., a CRISPR-transcription factor) to a target nucleic acid bound (hybridized) to a complementary gRNA sequence. Any scaffold sequence that comprises at least one stem loop structure and recruits a CRISPR protein may be used in the methods and agents described herein. Exemplary scaffold sequences will be evident to one of skill in the art and can be found for example in Jinek, et al. Science (2012) 337(6096):816-821, Ran, et al. Nature Protocols (2013) 8:2281-2308. PCT Application No. WO2014/093694, and PCT Application No. WO2013/176772.

In some embodiments, the gRNA sequence does not comprises a scaffold sequence and a scaffold sequence is expressed as a separate transcript. In some embodiments, the scaffold sequence is encoded on a nucleic acid (e.g., a vector) that also encodes the gRNA. In such embodiments, the gRNA sequence further comprises an additional sequence that is complementary to a portion of the scaffold sequence and functions to bind (hybridize) the scaffold sequence and recruit the CRISPR protein to the target nucleic acid.

It will be appreciated that a gRNA sequence, or portion thereof, is complementary to a target nucleic acid (e.g., a human homolog of a gene presented in Table 1) in a host cell if the gRNA sequence is capable of hybridizing to the target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid.

The region of the gRNA (approximately 12 nucleotides) that is adjacent to the protospacer adjacent motif (PAM) sequence, as described herein, may be referred to as a “seed region” of the gRNA. In some embodiments, the seed region of the gRNA sequence is at least 50%, 55%, 60%, 65%, 70%, 75%, 80%, 85%, 90%, 95%, 96%, 97%, 98%, 99%, or at least 100% complementary to the target nucleic acid. The remaining portion of the gRNA may be referred to as the “non-seed region” of the gRNA. In some embodiments, the non-seed region of the gRNA sequence is at least 50%, 55%, 60%, 65%, 70%, 75%, 80%, 85%, 90%, 95%, 96%, 97%, 98%, 99%, or at least 100% complementary to the target nucleic acid.

The gRNA sequence may be obtained from any source known in the art. For example, the gRNA sequence may be any nucleic acid sequence of the indicated length present in the nucleic acid of a host cell (e.g., genomic nucleic acid and/or extra-genomic nucleic acid). In some embodiments, gRNA sequences may be designed and synthesized to target desired nucleic acids, such as nucleic acids encoding transcription factors, signaling proteins, transporters, or proteins involved in a particular cellular process or belonging to a particular protein family.

In some embodiments, the gRNAs of the present disclosure have a length of 10 to 500 nucleotides. In some embodiments, a gRNA has a length of 10 to 20 nucleotides, 10 to 30 nucleotides, 10 to 40 nucleotides, 10 to 250 nucleotides, 10 to 300 nucleotides, 10 to 350 nucleotides, 10 to 400 nucleotides or 10 to 450 nucleotides. In some embodiments, a gRNA has a length of more than 500 nucleotides. In some embodiments, a gRNA has a length of 10, 15, 20, 15, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more nucleotides.

The terms “target nucleic acid,” “target site,” and “target sequence” may be used interchangeably throughout and refer to any nucleic acid sequence in a host cell that may be targeted by the gRNA sequences described herein. In some embodiments, the target nucleic acid is within the coding sequence of a human homolog of a gene provided in Table 1. In some embodiments, the target nucleic acid is not within the coding sequence of a human homolog of a gene provided in Table 1, such as within a regulatory region. In some embodiments, the target nucleic acid is not within the coding sequence of a human homolog of a gene provided in Table 1 and is not within a regulatory region. In some embodiments, the target nucleic acid is within an inhibitor of a human homolog of a gene provided in Table 1. In general, targeting of the target nucleic acid with the gRNAs described herein results in an enhancement of the expression and/or activity of a human homolog of a gene provided in Table 1.

The target nucleic acids are flanked on the 3′ side by a protospacer adjacent motif (PAM) that may interact with the CRISPR protein and be further involved in targeting the activity of the CRISPR protein to the target nucleic acid. It is generally thought that the nucleotide sequence of the PAM flanking the target nucleic acid depends on the CRISPR protein used and the source from which the endonuclease is derived. For example, for CRISPR protein that is a Cas9 endonuclease, or a variant of a Cas9 endonuclease, that is derived from Streptococcus pyogenes, the PAM sequence is NGG. For Cas9 endonucleases that are derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TIN.

In some embodiments, the agent is a gRNA and one or more additional agents, such as a CRISPR protein or nucleic acid encoding a CRISPR protein, may be administered to the subject and/or provided to a cell. In some embodiments, the gRNA and the CRISPR protein are administered as a preformed complex. In some embodiments, the CRISPR protein is a Cas endonuclease is a Cas9 enzyme or variant thereof. In some embodiments, the Cas9 endonuclease is derived from Streptococcus pyogenes, Staphylococcus aureus, Neisseria meningitidis, Streptococcus thermophilus, or Treponema denticola. In some embodiments, the nucleotide sequence encoding the Cas endonuclease may be codon optimized for expression in a host cell. In some embodiments, the endonuclease is a Cas9 homolog or ortholog.

In some embodiments, the nucleotide sequence encoding the Cas9 endonuclease is further modified to alter the activity of the protein. In some embodiments, the Cas9 endonuclease is a catalytically inactive Cas9. For example, dCas9 contains mutations of catalytically active residues (D10 and H840) and does not have nuclease activity. Alternatively or in addition, the Cas9 endonuclease may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g. CRISPR interference (CRISPRi)). In some embodiments, dCas9 is fused to a transcription factor or an activator domain therefrom, such as VP64 or VPR. CRISPR proteins comprising dCas9 fused to a transcription factor or domain therefrom are generally referred to as CRISPR-TF or CRISPR-transcription factors. Variant CRISPR-TF are also known in the art and may confer stronger transcriptional activation of a gene, as compared to a CRISPR-TF comprising, for example, dCas9-VP64. See, e.g., Chavez et al. Nat. Methods (2015) 12: 326-328; Farzadfard et al. ACS Synth. Biol. (2015) 517: 583-588; Tanenbaum Cell (2014) 159: 635-646. In some embodiments, such dCas9 fusion proteins are used with the constructs described herein for gene activation (e.g., CRISPR activation (CRISPRa)). See, e.g., Gilber et al. Cell. (2014) 159(3): 647-661. In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation. In some embodiments, dCas9 or Cas9 is fused to a Fok1 nuclease domain. In some embodiments, Cas9 or dCas9 fused to a Fok1 nuclease domain is used for genome editing.

Alternatively or in addition, the Cas endonuclease is a Cpf1 nuclease. In some embodiments, the host cell expresses a Cpf1 nuclease derived from Provetella spp. or Francisella spp. In some embodiments, the nucleotide sequence encoding the Cpf1 nuclease may be codon optimized for expression in a host cell.

Any of the nucleic acids, including nucleic acids encoding the proteins described herein, may be associated with or expressed from a recombinant expression vector. As used herein, a “vector” may be any of a number of nucleic acids into which a desired sequence or sequences may be inserted by restriction digestion and ligation or by recombination for transport between different genetic environments or for expression in a host cell. Vectors are typically composed of DNA, although RNA vectors are also available. Vectors include, but are not limited to: plasmids, fosmids, phagemids, virus genomes, and artificial chromosomes. In some embodiments, the vector is a lentiviral vector.

A recombinant expression vector is one into which a desired DNA sequence may be inserted, for example, by restriction digestion and ligation or recombination such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., galactosidase, fluorescence, luciferase or alkaline phosphatase), and genes which visibly affect the phenotype of transformed or transfected cells, hosts, colonies or plaques (e.g., green fluorescent protein, red fluorescent protein). Preferred vectors are those capable of autonomous replication and expression of the structural gene products present in the DNA segments to which they are operably joined.

In some embodiments, a gRNA, such as a gRNA that enhances expression and/or activity of a human homolog of any of the genes provided in Table 1, and a CRISPR protein are expressed on the same recombinant expression vector. In some embodiments, a gRNA and a CRISPR protein are expressed on two or more recombinant expression vectors.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule (e.g., nutrient, metabolite or drug). In some embodiments, the promoter is a galactose-inducible promoter (e.g., GAL1 promoter0. In some embodiments, the promoter is a doxycycline-inducible promoter.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator 5 sequences as desired. The vectors of the invention may optionally include 5′ leader or signal sequences. The choice and design of an appropriate vector is within the ability and discretion of one of ordinary skill in the art.

Recombinant expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. A nucleic acid molecule associated with the invention can be introduced into a cell or cells using methods and techniques that are standard in the art. For example, nucleic acid molecules can be introduced by standard protocols such as transformation including chemical transformation and electroporation, viral transduction, particle bombardment, etc. In some embodiments, the recombinant expression vector is introduced by viral transduction. In some embodiments, the viral transduction is achieved using a lentivirus. Expressing the nucleic acid molecule may also be accomplished by integrating the nucleic acid molecule into the genome.

Such a vector may be administered to a subject by a suitable method. Methods of delivering vectors are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6): 2082-2087); or viral transduction. In some embodiments, the vectors are administered to a subject, and thereby to the cells of the subject, by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of an agent, such as a nucleic acid agent or a protein agent, are retroviruses. In some embodiments, the vectors for expression of an agent, such as a nucleic acid agent or a protein agent, are lentiviruses. In some embodiments, the vectors for expression of an agent, such as a nucleic acid agent or a protein agent, are adeno-associated viruses.

In examples in which the vectors encoding an agent, such as a nucleic acid agent or a protein agent, are administered to the subject using a viral vector, viral particles that am capable of infecting cells of a subject and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to administration of the viral particles.

### Therapeutically Effective Amounts

In one aspect, the disclosure provides methods of treating a disorder associated with α-synuclein dysfunction with a therapeutically effective amount of an agent that enhances the expression and/or activity of a human homolog of one or more genes provided in Table 1. As used herein, a “therapeutically effective amount” and “effective amount,” which are used interchangeably herein, refer to an amount of an agent that is sufficient to improve or enhance at least one aspect of the disease or disorder. In some embodiments, the therapeutically effective amount is an amount that reduces one or more symptoms of the disorder, and/or enhances the survival of the subject having the disease or disorder. In some embodiments, the therapeutically effective amount of the agent is an amount effective in preventing or delaying the onset of a disorder associated with α-synuclein dysfunction or one or more symptoms thereof.

In some embodiments, the therapeutically effective amount is an amount that confers a neuroprotective effect in the subject. As used herein, the term “neuroprotective” or a “neuroprotective effect” refers to a reduction in the amount or rate of neurodegeneration. In some embodiments, the neuroprotective effect is suppression of cellular toxicity due to α-synuclein dysfunction.

An therapeutically effective amount of an agent can be selected by choosing among the various active compounds and weighing factors such as potency, relative bioavailability, subject body weight, severity of adverse side-effects and preferred mode of administration, in order to reduce or avoid inducing substantial toxicity and yet be effective in treating the particular subject.

The therapeutically effective amount of an agent can vary depending on such factors as the disorder or condition being treated, the particular agent(s) to be administered and properties thereof, the size of the subject, the gender of the subject, or the severity of the disorder. One of ordinary skill in the art can empirically determine the therapeutically effective amount of an agent without necessitating undue experimentation. In some embodiments, it is preferred that a maximum dose be used, that is, the highest safe dose according to some medical judgment. Multiple doses per day, week or month may be contemplated to achieve appropriate levels of the agent (e.g., systemic levels and/or local levels). In some embodiments, the agent that enhances the expression and/or activity of a human homolog of one or more genes provided in Table 1 is administered in a single dose. In some embodiments, the agent that enhances the expression and/or activity of a human homolog of one or more genes provided in Table 1 is administered in multiple doses, such as multiple doses administered concomitantly or sequentially. In some embodiments, 1, 2, 3, 4, 5, 6, 7, 8, 9, 10, or more than 10 doses of the agent are administered. In some embodiments, one or more loading doses of the agent is administered, following by one or more maintenance doses of the agent. In some embodiments, doses are administered at regular intervals while in other embodiments doses are administered at irregular intervals. In some embodiments, the agent is administered for an indefinite. Appropriate systemic levels of the agent can be determined by, for example, quantification of the agent in a blood or serum sample from the subject, assessing expression and/or activity of the gene enhanced by the agent. Any of the methods of administration can include monitoring levels of the agent, monitoring activity and/or expression, assessing any one or more symptoms of the disorder, and dose adjustment as needed.

In some embodiments, the agent is administered with one or more additional agents, such as therapeutic agents. The additional agents can be administered before, simultaneously, or after administration of the agent. In some embodiments, 2, 3, 4, 5, or more additional agents are administered.

In some embodiments, more than one agent that enhances the expression and/or activity of a human homolog of one or more genes provided in Table 1 are administered to the subject. In some embodiments, at least 2, 3, 4, 5, or more agents that enhance the expression and/or activity of a human homolog of one or more genes provided in Table 1 are administered to the subject. In some embodiments, the more than one agents are administered to the subject at the same time, for example in a combined dose.

In some embodiments, when more than one agent is administered to the subject at different times, for example a first agent is administered to the subject and a second agent is administered to the subject at a subsequent time. In some embodiments, the amount of a therapeutically effective amount of an agent administered in combination with one or more additional agents is less than the therapeutically effective amount of the agent when administered in the absence of an additional agent.

In methods for treating neurodegenerative disorders associated with α-synuclein dysfunction in a subject, a therapeutically effective amount of an agent is any amount that provides a beneficial effect in the subject, such as a neuroprotective effect. In some embodiments, the therapeutically effective amount of the agent reduces or prevents neurodegeneration, including cell death of neurons. In some embodiments, the therapeutically effective amount of an agent that enhances expression and/or activity of any the genes described herein is reduced when the agent is administered concomitantly or sequentially with any one or more additional agents as compared to the effective amount of the agent when administered in the absence of the additional agent(s). In some embodiments, the effective amount of an agent that enhances expression and/or activity of a human homolog of one or more genes provided in Table 1 is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, 5.0-, 10.0-, 15.0-, 20.0-, 25.0-, 30.0-, 35.0-, 40.0-, 45.0-, 50.0-, 55.0-, 60.0-, 65.0-, 70.0-, 75.0-, 80.0-, 85.0-, 90.0-, 95.0-, 100-, 200-, 300-, 400-, or at least 500-fold or more when the agent is concomitantly or sequentially administered with one or more additional agents (e.g., combinations of two agents that enhance expression and/or activity of human homologs of the same or different genes presented in Table 1).

In some embodiments, the therapeutically effective amount of an agent is an amount sufficient to reduce neurodegeneration, including cell death of neurons, by at least 10%, at least 20%, at least 30%, at least 40% at least 50%, at least 60%, at least 70%, at least 80%, at least 90%, at least 95%, at least 96%, at least 97%, at least 98%, or at least 99% compared to neurodegeneration in the absence of the agent. In some embodiments, the therapeutically effective amount of an agent is an amount sufficient to reduce neurodegeneration or one or more symptoms of the neurodegenerative disorder by at least 10%, at least 20%, at least 30%, at least 40% at least 50%, at least 60%, at least 70%, at least 80%, at least 90%, at least 95%, at least 96%, at least 97%, at least 98%, or at least 99% compared to the severity of the symptom in the absence of the agent.

### Administration

The methods described herein involve treating a neurodegenerative disorder associated with α-synuclein dysfunction comprising administering to the subject an agent that enhances the expression and/or activity of a human homolog of one or more of the genes provided in Table 1. As used herein “treating” can include: improving one or more symptoms of a disorder; curing a disorder, preventing a disorder from becoming worse; slowing the rate of progression of a disorder; or preventing a disorder from re-occurring.

Aspects of the present disclosure provide methods of treating a neurodegenerative disorder associated with α-synuclein dysfunction in a subject. In some aspects, the methods provide a neuroprotective and disease-modifying treatment of a neurodegenerative disorder associated with α-synuclein dysfunction. In some embodiments, the subject is a subject having, suspected of having, or at risk of developing a disorder associated with α-synuclein dysfunction. In some embodiments, the subject is a subject having, suspected of having, or at risk of developing a neurodegenerative disorder associated with α-synuclein dysfunction. In some embodiments, the subject is a mammalian subject, including but not limited to a dog, cat, horse, cow, pig, sheep, goat, rodent, or primate. In some embodiments, the subject is a human subject, such as a human patient. The terms “patient,” “subject,” or “individual” may be used interchangeably and refer to a subject who is in need of the treatment as described herein. Such a subject may exhibit one or more symptoms associated with the neurodegenerative disorder. Alternatively or in addition, such a subject may carry or exhibit one or more risk factors for the neurodegenerative disorder. In some embodiments, the subject has been diagnosed with a disorder associated with α-synuclein dysfunction. In some embodiments, the subject has been diagnosed with Parkinson's disease.

In some embodiments, the agent is administered orally, parenterally, intravenously, topically, intraperitoneally, subcutaneously, intracranially, intrathecally, or by inhalation. In some embodiments, the agent is administered by continuous infusion. Selection of an appropriate route of administration will depend on various factors not limited to the particular disorder and/or severity of the disorder.

In some embodiments, the agent is administered in one dose. In some embodiments, the agent is administered in multiple doses. In some embodiments, more than one agent (e.g., 2, 3, 4, 5, or more agents) are administered together in one dose. In some embodiments, more than one agent (e.g., 2, 3, 4, 5, or more agents) are administered in separate doses. In some embodiments, the multiple or separate doses are administered by the same route of administration (e.g., each dose is administered intravenously). In some embodiments, the multiple or separate doses are administered by different routes of administrations (e.g., one dose is administered intravenously and another dose(s) is administered orally).

Any agent that enhances expression and/or activity of a human homolog of one or more of the genes provided in Table 1 can be administered to a subject as a pharmaceutical compositions, which may routinely contain pharmaceutically acceptable concentrations of salt, buffering agents, preservatives, compatible carriers, adjuvants, pharmaceutically acceptable excipients, and optionally other therapeutic ingredients. The nature of the pharmaceutical carrier, excipient, and other components of the pharmaceutical composition will depend on the mode of administration. The pharmaceutical compositions of the disclosure may be administered by any means and route known to the skilled artisan in carrying out the treatment methods described herein.

Any of the agents, described herein, that enhances expression and/or activity of a human homolog of one or more of the genes provided in Table 1 may be delivered systemically. In some embodiments, the agent is formulated for parenteral administration by injection. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form.

Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes.

Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In some embodiments, the agent is formulated for oral administration. In some embodiments, the agent is formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated.

Pharmaceutical preparations for oral administration can be obtained as solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on Tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

Any of the agents described herein may be provided in the formulation as fine multiparticulates in the form of granules or pellets. The formulation of the material for capsule administration could also be as a powder, lightly compressed plugs or even as tablets. The pharmaceutical composition could be prepared by compression. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500. Emcompress and Avicell. Disintegrants may be included in the formulation of the pharmaceutical composition, such as in a solid dosage form. Materials used as disintegrants include but are not limited to starch, including the commercial disintegrant based on starch, Explotab®, sodium starch glycolate, Amberlite, sodium carboxymethylcellulose, ultramylopectin, sodium alginate, gelatin, orange peel, acid carboxymethyl cellulose, natural sponge and bentonite may also be used. Binders may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Glidants that might improve the flow properties of the drug during formulation and to aid rearrangement during compression might be added. The glidants may include starch, talc, pyrogenic silica and hydrated silicoaluminate.

For administration by inhalation, the agent may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas.

Also contemplated herein is pulmonary delivery of an agent that enhances expression and/or activity of a human homolog of one or more genes provided in Table 1. The agent may be delivered to the lungs of a mammal for local or systemic delivery. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13:143-146 (endothelin-1); Hubbard et al., 1989, Annals of Internal Medicine, Vol. III, pp. 206-212 (a1-antitrypsin): Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569.

Nasal delivery of a pharmaceutical composition comprising an agent that enhances the expression and/or activity of a human homolog of one or more genes provided in Table 1 is also contemplated. Nasal delivery allows the passage of a pharmaceutical composition to the blood stream directly after administering the composition to the nose, without the necessity for deposition of the product in the lung.

In some embodiments, the agent is administered locally. Local administration methods are known in the art and will depend on the target area or target organ. Local administration routes include the use of standard topical administration methods such by inhalation, intracranially, and/or intrathecally. In some embodiments, any of the agents described herein may be delivered locally, for example to the site of cells having α-synuclein dysfunction. In some embodiments, any of the agents described herein may be delivered to the nervous system. In some embodiments, any of the agents described herein may be delivered by intracranial injection. In some embodiments, any of the agents described herein may be delivered through the spinal cord. The agents may also be formulated in rectal or vaginal compositions such as suppositories or retention enemas, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble analogs, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose analogs, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or one or more auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference. The agents and compositions described herein may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The pharmaceutical compositions of the disclosure contain an effective amount of an agent with a pharmaceutically-acceptable carrier or excipient. The term pharmaceutically acceptable excipient means one or more compatible solid or liquid filler, diluents or encapsulating substances which are suitable for administration to a human or other vertebrate animal. The term excipient denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

Both non-biodegradable and biodegradable polymeric materials can be used in the manufacture of particles for delivering the compositions of the disclosure. Such polymers may be natural or synthetic polymers. The polymer is selected based on the period of time over which release is desired. Bioadhesive polymers of particular interest include bioerodible hydrogels described by Sawhney et al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(5 octadecyl acrylate). The agents described herein may be contained in controlled release systems. The term “controlled release” is intended to refer to any agents and compositions described herein containing formulation in which the manner and profile of agents and compositions described herein release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound therefrom. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Screening Methods

Also provided herein are methods for identifying a genetic network involved in regulating a cellular response, such as suppressing α-synuclein toxicity. In some embodiments, the methods may be used to identify a genetic network involved in a complex genetic disorder (e.g., Alzheimer's disease) or a cellular stress response that involves a genetic network.

The methods involve expressing a plurality of randomized guide RNAs and a CRISPR protein, such as any of the CRISPR proteins described herein, in a population of cells and culturing the population of cells under conditions that induce the cellular response. Subpopulations of cells having an altered readout of the cellular response from the population of cells may be isolated and used to identify randomized gRNAs that are present in the subpopulation of cells as gRNAs that regulates a transcriptional network involved in the cellular response. In some embodiments, the CRISPR protein is CRISPR-Cas-based transcription factor, such as dCas9-VP64 or variants thereof.

Any cellular response that can be assessed may be used in the methods described herein. In some embodiments, the cellular response is a cellular response to induction of synuclein protein, such as α-synuclein, β-synuclein, or γ-synuclein. Each of α-synuclein, β-synuclein, or γ-synuclein are thought to be involved in the pathogenesis and/or pathology of neurodegenerative diseases. High levels of expression of synuclein proteins or expression of mutated synuclein proteins may result in aggregation of synclein protein, which, at least in the case of α-synuclein, may induce toxicity (cell death) of cells, including neurons. Assessing such a cellular response may involve subjecting the population of cells expressing the plurality of randomized gRNA to the cellular response (e.g., synuclein toxicity) and isolating cells that survive. The gRNAs that are expressed in the cells that survived are identified as gRNAs that conferred a protective effect and suppressed toxicity. In some embodiments, the synuclein toxicity may be induced by enhancing expression of a synuclein protein or by expressing a mutant synuclein protein.

The methods described herein involve identifying and/or isolating subpopulations of cells having an altered readout of the cellular response. As used herein, an “altered readout” refers to an enhanced or a reduced response to the cellular response as compared to a control cell or a control population of cells. A readout encompasses any observable and/or quantifiable phenotype of a cellular response. In some embodiments, the cellular response is α-synuclein toxicity and the altered readout is reduced α-synuclein toxicity, as compared to α-synuclein toxicity in a control population of cells.

As used herein, a nucleotide sequence of a gRNA or a portion thereof (e.g., the seed region or non-seed region) is considered to be randomized, if each the nucleotide present (A, T, C, or G) at each position of the sequence is selected in an unbiased manner. In some embodiments, a portion of the gRNA is randomized and a portion of the gRNA is not randomized. For a portion of a gRNA that is not randomized, the nucleotide sequence may be selected to have desired characteristics or binding or structural properties.

In some embodiments, the nucleotide sequence of a gRNA, or a portion thereof, is pseudo-randomized. As used herein, the term “pseudo-randomized” refers to a process of selecting particular positions of the gRNA that are randomized and other positions are not randomized. In some embodiments, one or more particular nucleotides are weighted at a particular position of the gRNA, meaning the particular nucleotides are present more frequently at the particular position(s) are compared to other nucleotides.

In some embodiments, the content of guanine and cytosine nucleotides (the GC content) of the randomized gRNAs may be selected depending on the GC content of the genome of the cell (or organism from which the cell was derived). In some embodiments, the GC content of the gRNA is between 50%-70%, 60%-70%, 55%-65%, 50%-55%, 65%-75%. In some embodiments, the GC content of the gRNA is about 50%, 51%, 52%, 53%, 54%, 55%, 56%, 57%, 58%, 59%, 60%, 61%, 62%, 63%, 64%, 65%, 66%, 67%, 68%, 69%, 70% or more.

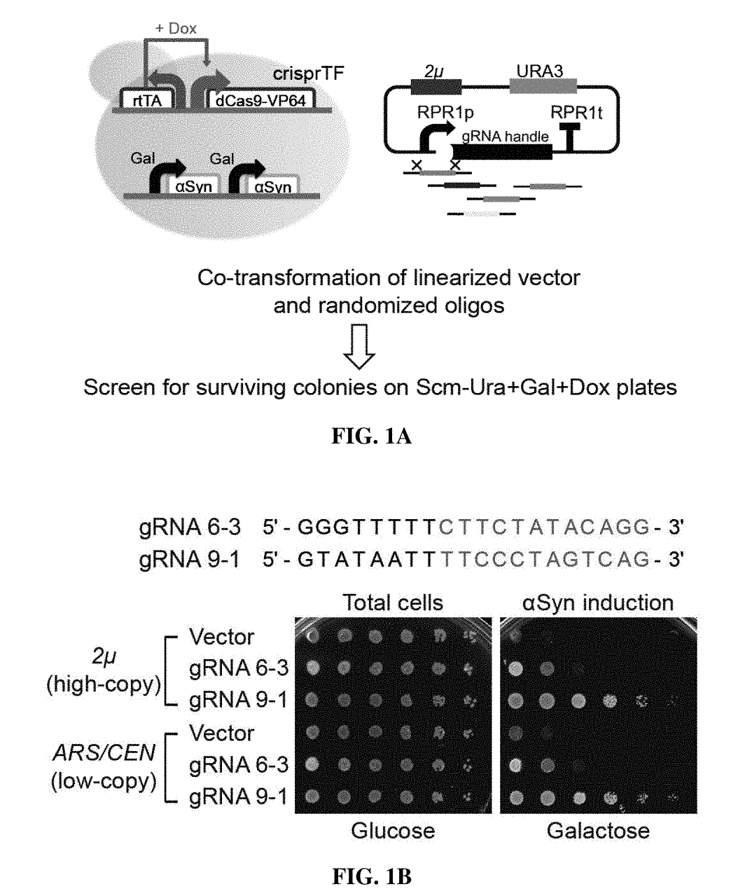

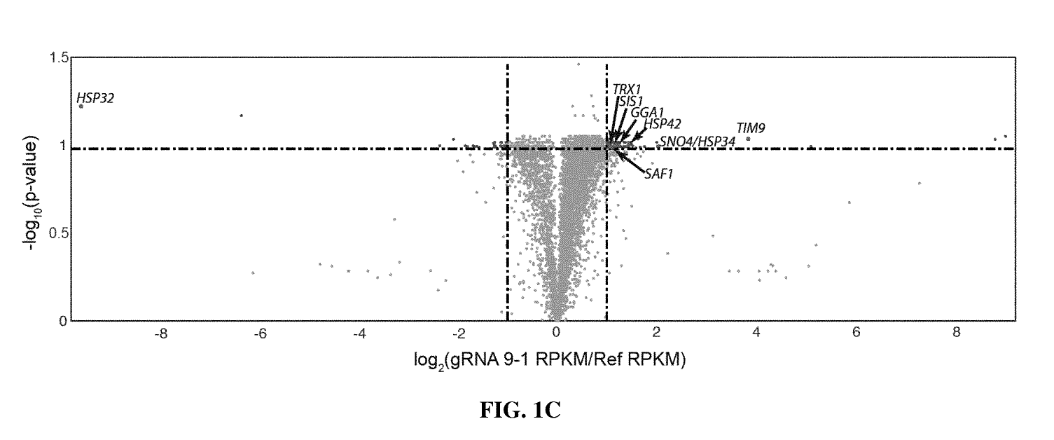

To identify target genes perturbed by the randomized gRNA, transcriptome profiling was performed (see, for example, FIG. 1C and description thereof). This method enriched for genes differentially expressed in cells exposed to the gRNA versus control cells not exposed to the gRNA.

The invention is not limited in its application to the details of construction and the arrangement of components set forth in the description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, particularly for the teachings referenced herein.

### Example

Randomized CRISPR-Cas Transcriptional Perturbation Screening Identifies Individual and Combinations of Genes that Protect Against Alpha-Synuclein Toxicity

The genome-wide perturbation of transcriptional networks with CRISPR-Cas technology has primarily involved systematic and targeted gene modulation. As described herein, a complementary and distinct high-throughput screening platform was developed based on randomized CRISPR-Cas transcription factors (crisprTFs) that introduce global perturbations within transcriptional networks. This technology was applied to a yeast model of Parkinson's disease (PD) and used to identify guide RNAs (gRNAs) that modulated transcriptional networks and protected cells from alpha-synuclein (αSyn) toxicity. Global gene expression profiling revealed a substantial number of genes that were differentially modulated by a strong protective gRNA. These genes were validated to rescue yeast from αSyn toxicity and associated defects when over-expressed. The genes identified as regulated by the protective gRNA belong to families involved in a diverse set of processes, including protein quality control, ER/Golgi trafficking, lipid metabolism, mitochondrial function, and stress response. Human homologs of highly ranked hits were further verified in a human neuronal PD model to synergistically protect against αSyn-induced cell death. These results demonstrate that the methods described herein, such as the high-throughput and unbiased perturbation of transcriptional networks via randomized crisprTFs, are effective tools for studying complex biological phenotypes and discovering novel disease modulators.

Due to conserved molecular mechanisms and the availability of genetic tools, Saccharomyces cerevisiae is a useful model system to systematically study and identify genes involved in neurodegenerative diseases such as PD and Alzheimer's Disease (39, 44-53). Aggregation of misfolded αSyn in intraneuronal Lewy bodies has been shown to be one of the pathological hallmarks of Parkinson's Disease (PD) (34, 35). Overexpression of αSyn in different eukaryotic model organisms has been used to elucidate the complex cellular processes associated with PD (36-44). The methods described herein can be used to identify genetic networks, such as transcriptional networks, involved in complex genetic disorders like Parkinson's Disease using a S. cerevisiae model of the disorder.

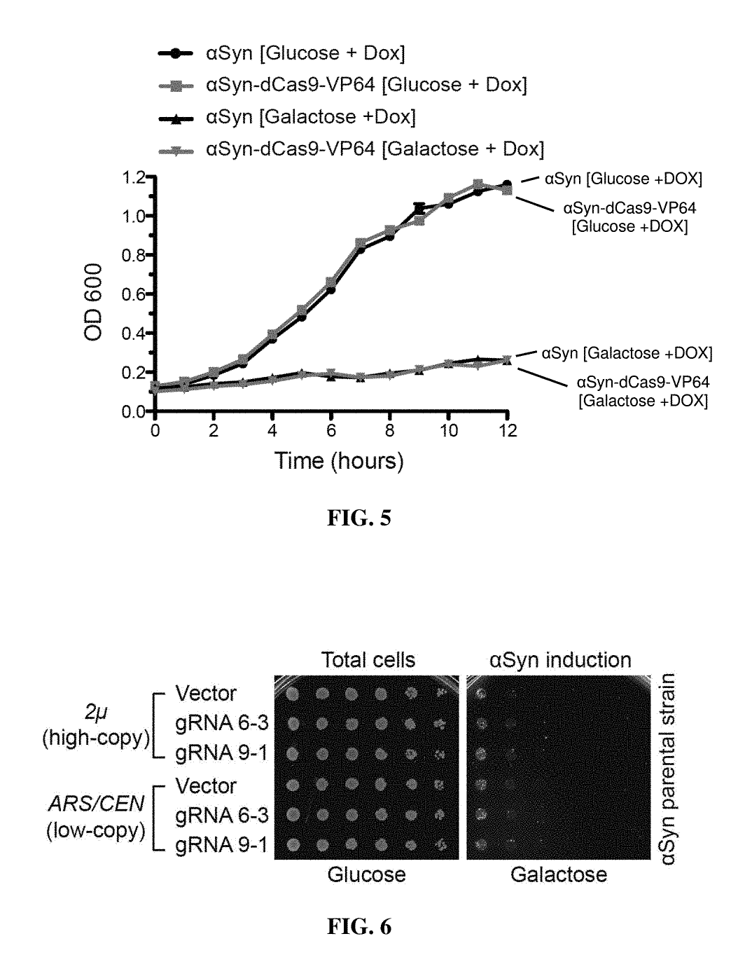

A crisprTF (dCas9-VP64) expression cassette was cloned under the control of a Doxycycline (Dox)-inducible (Tet-ON) promoter. To build the yeast strain used in the screening methods described herein, the crisprTF construct was integrated into the genome of an αSyn-expressing S. cerevisiae strain (referred to as the yeast parental strain), which over-expresses two copies of human wild-type αSyn (SNCA) gene fused to yellow fluorescent protein (YFP) under the control of a galactose (Gal)-inducible promoter (54) (FIG. 1A). Both the parental and the screen strains showed significant cellular growth defects in presence of galactose due to over-expression of αSyn. The expression of dCas9-VP64 with no gRNA in the screen strain did not interfere with normal cellular growth or αSyn-associated toxicity (FIG. 5).

A randomized gRNA-expressing plasmid library was constructed by co-transforming into a S. cerevisiae strain a linearized high-copy 2μ plasmid, flanked by the RPR1 promoter (RPR1p), and gRNA handle at the ends, with a randomized oligo library encoding 20-mer randomized nucleotides flanked by homology arms to the ends of the vector. After transformation of the library, cells were recovered in liquid culture with Dox (1 μg/mL) for 12 hours to amplify the library and induce crisprTF expression. The cultures were then plated on synthetic complete media (Scm)−Uracil (Ura)+Gal+Dox plates, and gRNAs from surviving colonies were characterized by colony PCR followed by Sanger sequencing (FIG. 1A).

To validate activity of the identified gRNAs, each candidate gRNA was re-cloned in both high-copy 2μ and low-copy ARS/CEN plasmids, and transformed back into both the parental and screen strain. Two gRNAs (designated as gRNA 6-3 and 9-1) expressed from either high-copy and low-copy plasmids were validated and found to rescue the screen strain from αSyn toxicity (FIG. 1B). gRNA 6-3 (SEQ ID NO: 2) is a moderate suppressor of αSyn toxicity whereas gRNA 9-1 (SEQ ID NO: 1), which was identified in two independent screens, is a strong αSyn suppressor and was thus chosen for further characterization.

Although no perfect match was predicted between the identified gRNAs and the yeast genome, a relaxed search criteria (up to two mismatches inside the seed region) revealed the presence of a few dozen sites that could potentially serve as off-target binding sites of these gRNAs, including one in the GAL4 gene (Table 2).

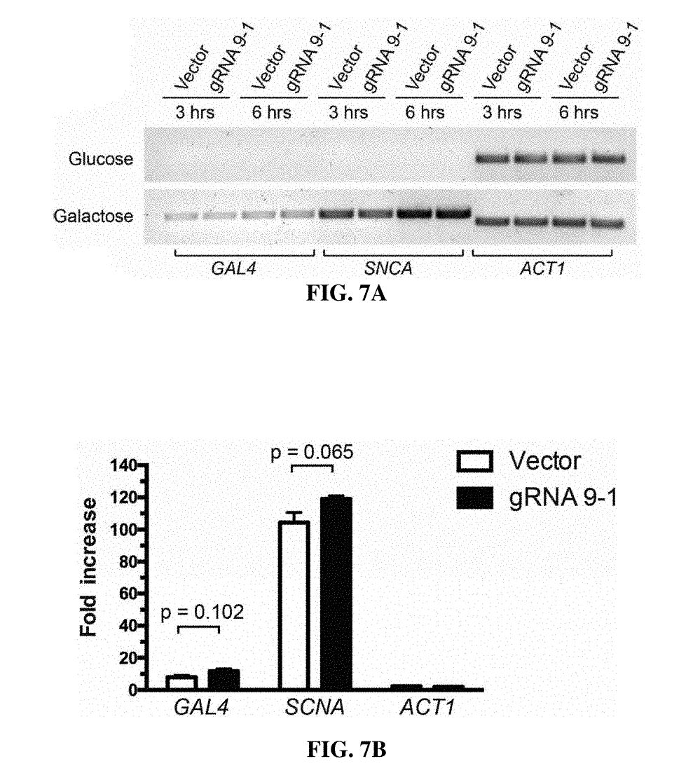

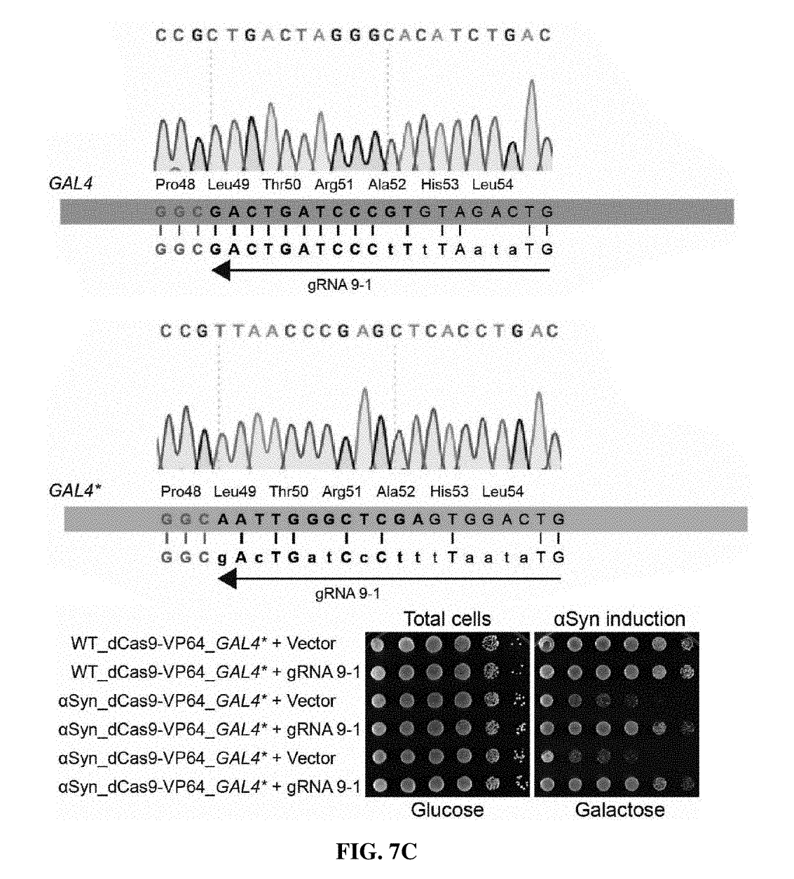

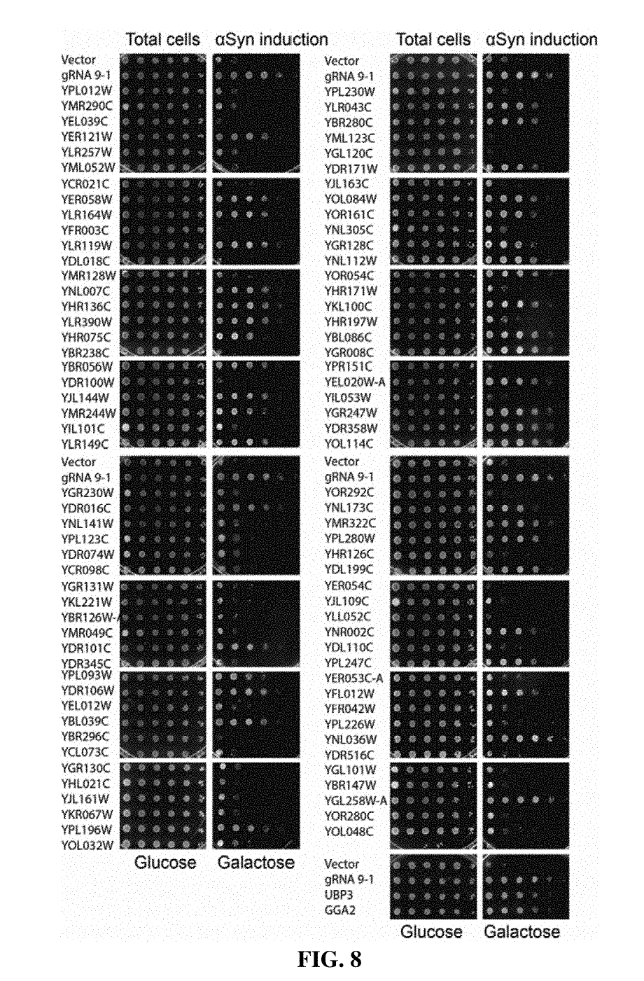

As additional controls, it was confirmed that the gRNA-mediated suppression of αSyn toxicity depended on the presence of dCas9-VP64 (FIG. 6) and that GAL4 and αSyn (SNCA) expression levels were not directly affected by gRNA 9-1/crisprTF (FIGS. 7A and 7B). GAL4 acts as the activator of the GAL) promoter, which drives expression of αSyn. To further confirm that the protective effect observed with gRNA 9-1 was not due to repression of GAL4, the putative gRNA 9-1 off-target binding site predicted in GAL4 was modified such that there were only five matches in the seed sequence (GAL4*). Even with the modified GAL4 locus, gRNA 9-1 preserved its ability to rescue the screen yeast strain from αSyn toxicity (FIG. 7C).

TABLE 2
Predicted binding sites for gRNA 6-3 and gRNA 9-1 in the S. cerevisiae genome

| | | | | Number of | | | |
|---|---|---|---|---|---|---|---|
| | | | | Mismatch in | Total Number | | Systematic |
| gRNA | Target Site | Target Sequence* | PAM | Seed Region | of Mismatch | Gene Name | Name |
| gRNA6-3 | I: 115314- | GGtaaTgaCTTCTtgAC | NGG | 2 | 7 | YAL019W-A | YAL019W-A |
| | 115337: − | AGG-TGGC (SEQ ID | | | | | |
| | | NO: 17) | | | | | |
| gRNA6-3 | II: 141581- | ttcaacaT_CTTCTgTACg | NAG | 2 | 9 | PRE7 | YBL041W |
| | 141604: + | GG-AAGA (SEQ ID | | | | | |
| | | NO: 18) | | | | | |
| gRNA6-3 | II: 212896- | atGaaTac_CTTCaATAC | NGG | 2 | 8 | SLA1 | YBL007C |
| | 212919: − | tGG-TGGT (SEQ ID | | | | | |
| | | NO: 19) | | | | | |
| gRNA6-3 | II: 447874- | aGGggaaT_CTTgTATA | NAG | 2 | 7 | SIF2 | YBR103W |
| | 447897: + | CAGa-AAGT (SEQ ID | | | | | |
| | | NO: 20) | | | | | |
| gRNA6-3 | II: 524119- | GatTaTTg_CTTCTATAt | NNGG | 2 | 6 | IRA1 | YBR140C |
| | 524142: − | tGG-ATGG (SEQ ID | | | | | |
| | | NO: 21) | | | | | |
| gRNA6-3 | III: 164910- | tttaaaga_CTTCTATAgA | NAG | 2 | 10 | NPP1 | YCR026C |
| | 164933: − | tG-AAGA (SEQ ID NO: | | | | | |
| | | 22) | | | | | |
| gRNA6-3 | III: 210476- | ttcTTcTT_CTTCTtTACt | NAG | 2 | 6 | IMG1 | YCR046C |
| | 210499: + | GG-GAGT (SEQ ID NO: | | | | | |
| | | 23) | | | | | |
| gRNA6-3 | IV: 1723- | GctTTTcg_CTTtTATAC | NGG | 2 | 6 | COS7 | YDL248W |
| | 1746: − | AGc-AGGA (SEQ ID | | | | | |
| | | NO: 24) | | | | | |
| gRNA6-3 | IV: 17896- | ttcgggTa_CTTCTAaAC | NGG | 2 | 9 | AAD4 | YDL243C |
| | 17919: − | AGa-CGGA (SEQ ID | | | | | |
| | | NO: 25) | | | | | |
| gRNA6-3 | IV: 112128- | tatcggaa_aTTCTtTACA | NAG | 2 | 10 | SNF3 | YDL194W |
| | 112151: + | GG-TAGG (SEQ ID NO: | | | | | |
| | | 26) | | | | | |
| gRNA6-3 | IV: 145433- | tGtagcac_CTTCTATAg | NAG | 1 | 8 | AIR2 | YDL175C |
| | 145456: − | AGG-AAGT (SEQ ID | | | | | |
| | | NO: 27) | | | | | |
| gRNA6-3 | IV: 309773- | tGcaaTTT_CTTCTAaA | NNGG | 2 | 6 | RPL13A | YDL082W |
| | 309796: + | CAGt-GCGG (SEQ ID | | | | | |
| | | NO: 28) | | | | | |
| gRNA6-3 | IV: 354026- | GatTggag_CaTCTATAt | NAG | 2 | 8 | MBP1 | YDL056W |
| | 354049: − | AGG-GAGC (SEQ ID | | | | | |
| | | NO: 29) | | | | | |
| gRNA6-3 | IV: 579322- | GatTTcca_CTTCTgTAC | NGG | 2 | 7 | AIM7 | YDR063W |
| | 579345: + | AGa-TGGA (SEQ ID | | | | | |
| | | NO: 30) | | | | | |
| gRNA6-3 | IV: 588552- | tcccTTag_aTTCTgTAC | NAG | 2 | 8 | EMP16 | YDR070C |
| | 588575: + | AGG-AAGA (SEQ ID | | | | | |
| | | NO: 31) | | | | | |
| gRNA6-3 | IV: 700228- | tttcTcag_CTTaTATAaA | NNGG | 2 | 9 | YDR124W | YDR124W |
| | 700251: + | GG-ATGG (SEQ ID NO: | | | | | |
| | | 32) | | | | | |
| gRNA6-3 | IV: 754827- | cGGaggaa_CTTCaATAg | NAG | 2 | 8 | KGD2 | YDR148C |
| | 754850: + | AGG-TAGA (SEQ ID | | | | | |
| | | NO: 33) | | | | | |
| gRNA6-3 | IV: 780586- | GttTgTcT_CTTaTATAC | NGG | 2 | 6 | YDR161W | YDR161W |
| | 780609: − | AGc-CGGA (SEQ ID | | | | | |
| | | NO: 34) | | | | | |
| gRNA6-3 | IV: 825659- | ataTccgg_CTTCTtTgCA | NAG | 2 | 9 | SCC2 | YDR180W |
| | 825682: − | GG-GAGT (SEQ ID NO: | | | | | |
| | | 35) | | | | | |
| gRNA6-3 | IV: 868491- | GttTaacT_CTTCaATAg | NNGG | 2 | 7 | MSS4 | YDR208W |
| | 868514: − | AGG-TCGG (SEQ ID | | | | | |
| | | NO: 36) | | | | | |
| gRNA6-3 | IV: 1032570- | cGtgggTT_CTTCTATA | NAG | 1 | 6 | ZIP1 | YDR285W |
| | 1032593: + | gAGG-GAGA (SEQ ID | | | | | |
| | | NO: 37) | | | | | |
| gRNA6-3 | IV: 1174819- | ctcaTaTa_tTTgTATACA | NAG | 2 | 8 | | |
| | 1174842: + | GG-AAGA (SEQ ID | | | | | |
| | | NO: 38) | | | | | |
| gRNA6-3 | IV: 188425- | tGaaaTca_CTTCTtTAC | NGG | 2 | 8 | SPC110 | YDR356W |
| | 1188448: 4 | AaG-AGGA (SEQ ID | | | | | |
| | | NO: 39) | | | | | |
| gRNA6-3 | IV: 1195737- | aacgTaaT_CTTgTATAa | NGG | 2 | 8 | BCP1 | YDR361C |
| | 1195760: + | AGG-TGGA (SEQ ID | | | | | |
| | | NO: 40) | | | | | |
| gRNA6-3 | IV: 1197663- | tttgaaaa_tTTgTATACA | NGG | 2 | 10 | TFC6 | YDR362C |
| | 1197686: + | GG-AGGA (SEQ ID | | | | | |
| | | NO: 41) | | | | | |
| gRNA6-3 | IV: 1266351- | tttagaag_CTTCTATtCAa | NAG | 2 | 10 | SXM1 | YDR395W |
| | 1266374: + | G-AAGA (SEQ ID NO: | | | | | |
| | | 42) | | | | | |
| gRNA6-3 | IV: 1320005- | taaaTaga_CaTCTATAC | NAG | 2 | 9 | DYN2 | YDR424C |
| | 1320028: + | AcG-TAGT (SEQ ID | | | | | |
| | | NO: 43) | | | | | |
| gRNA6-3 | IV: 1385972- | tatgcTTc_CcTCcATAC | NAG | 2 | 8 | YDR461C-A | YDR461C-A |
| | 1385995: − | AGG-CAGG (SEQ ID | | | | | |
| | | NO: 44) | | | | | |
| gRNA6-3 | IX: 102306- | cctTcaaT_CTTCTATAg | NGG | 2 | 8 | FKH1 | YIL131C |
| | 102329: + | AGC-CGGT (SEQ ID | | | | | |
| | | NO: 45) | | | | | |
| gRNA6-3 | IX: 187934- | tGtTTTTa_aTTaTATAC | NNGG | 2 | 5 | LYS12 | YIL094C |
| | 187957: + | AGG-TTGG (SEQ ID | | | | | |
| | | NO: 46) | | | | | |
| gRNA6-3 | IX: 237025- | acGagTca_CTTCTATAa | NAG | 2 | 8 | YIL067C | YIL067C |
| | 237048: + | gGG-TAGG (SEQ ID | | | | | |
| | | NO: 47) | | | | | |
| gRNA6-3 | V: 20258- | aaccTTgT_CTTCaATcC | NGG | 2 | 7 | DSF1 | YEL070W |
| | 20281: − | AGG-CGGC (SEQ ID | | | | | |
| | | NO: 48) | | | | | |
| gRNA6-3 | V: 133587- | taaggaaa_CTTCTAaAC | NNGG | 2 | 10 | GLC3 | YEL011W |
| | 133610: + | AGt-TCGG (SEQ ID | | | | | |
| | | NO: 49) | | | | | |
| gRNA6-3 | V: 192917- | tttcTgac_CTTCaATACA | NGG | 2 | 9 | SPC25 | YER018C |
| | 192940: + | tG-GGGG (SEQ ID NO: | | | | | |
| | | 50) | | | | | |
| gRNA6-3 | V: 263157- | cctgacTT_tTTaTATACA | NGG | 2 | 8 | GIP2 | YER054C |
| | 263180: + | GG-TGGC (SEQ ID NO: | | | | | |
| | | 51) | | | | | |
| gRNA6-3 | V: 282867- | tcGaggcT_CTTCTtTAC | NAG | 2 | 8 | YER064C | YER064C |
| | 282890: + | cGG-GAGT (SEQ ID | | | | | |
| | | NO: 52) | | | | | |
| gRNA6-3 | V: 391439- | cctTaaac_CTTCTATAa | NAG | 2 | 9 | BOI2 | YER114C |
| | 391462: + | AtG-CAGA (SEQ ID | | | | | |
| | | NO: 53) | | | | | |
| gRNA6-3 | V: 393983- | tttcaacg_CTTCTAaAtA | NAG | 2 | 10 | BOI2 | YER114C |
| | 394006: + | GG-GAGA (SEQ ID | | | | | |
| | | NO: 54) | | | | | |
| gRNA6-3 | VI: 79298- | atacaTaT_tTTCTATAC | NGG | 1 | 7 | CAK1 | YFL029C |
| | 79321: + | AGG-GGGT (SEQ ID | | | | | |
| | | NO: 55) | | | | | |
| gRNA6-3 | VI: 232258- | atGaTTcT_CTTCTATAt | NAG | 1 | 5 | IRC5 | YFR038W |
| | 232281: + | AGG-CAGG (SEQ ID | | | | | |
| | | NO: 56) | | | | | |
| gRNA6-3 | VI: 243249- | aacgTggT_CTaCTATAC | NGG | 1 | 7 | YFR045W | YFR045W |
| | 243272: − | AGG-AGGA (SEQ ID | | | | | |
| | | NO: 57) | | | | | |
| gRNA6-3 | VII: 15025- | taaTaacc_CTTtTATACA | NNGG | 2 | 9 | ADH4 | YGL256W |
| | 15048: − | tG-TTGG (SEQ ID NO: | | | | | |
| | | 58) | | | | | |
| gRNA6-3 | VII: 15750- | aacaTaag_CTTCgATAC | NAG | 2 | 9 | ADH4 | YGL256W |
| | 15773: − | AGt-GAGT (SEQ ID | | | | | |
| | | NO: 59) | | | | | |
| gRNA6-3 | VII: 158407- | tctcaTTc_tTTCTATAaA | NGG | 2 | 8 | GTS1 | YGL181W |
| | 158430: + | GG-GGGC (SEQ ID | | | | | |
| | | NO: 60) | | | | | |
| gRNA6-3 | VII: 385070- | cataTaTa_CTTaTATAC | NAG | 2 | 8 | PUS2 | YGL063W |
| | 385093: − | AGc-GAGA (SEQ ID | | | | | |
| | | NO: 61) | | | | | |
| gRNA6-3 | VII: 780897- | aaccaaaT_CTTCaAcAC | NAG | 2 | 9 | THI4 | YGR144W |
| | 780920: − | AGG-TAGC (SEQ ID | | | | | |
| | | NO: 62) | | | | | |
| gRNA6-3 | VII: 1023603- | tGtcagTg_CTTCTAaAC | NNGG | 2 | 8 | YGR266W | YGR266W |
| | 1023626: + | AaG-ATGG (SEQ ID | | | | | |
| | | NO: 63) | | | | | |
| gRNA6-3 | VII: 1030947- | GaaTcTcc_tTTtTATAC | NNGG | 2 | 7 | YTA7 | YGR270W |
| | 1030970: − | AGG-TTGG (SEQ ID | | | | | |
| | | NO: 64) | | | | | |
| gRNA6-3 | VII: 1045389- | atccaaga_CTTCTgTAC | NGG | 2 | 10 | RNH70 | YGR276C |
| | 1045412: − | AaG-AGGA (SEQ ID | | | | | |
| | | NO: 65) | | | | | |
| gRNA6-3 | VIII: 19909- | atccaaTT_CTTCcATAtA | NNGG | 2 | 8 | ARN1 | YHL040C |
| | 19932: − | GG-CTGG (SEQ ID NO: | | | | | |
| | | 66) | | | | | |
| gRNA6-3 | VIII: 76355- | ttaaTTag_CTTCTtTACA | NGG | 2 | 8 | YLF2 | YHL014C |
| | 76378: − | tG-CGGC (SEQ ID NO: | | | | | |
| | | 67) | | | | | |
| gRNA6-3 | VIII: 145978- | tGaccTTc_aTTCaATAC | NNGG | 2 | 7 | YHR020W | YHR020W |
| | 146001: − | AGG-TTGG (SEQ ID | | | | | |
| | | NO: 68) | | | | | |
| gRNA6-3 | VIII: 231050- | ttcgTTgc_CTTtgATACA | NAG | 2 | 8 | SSF1 | YHR066W |
| | 231073: − | GG-GAGT (SEQ ID NO: | | | | | |
| | | 69) | | | | | |
| gRNA6-3 | VIII: 300415- | ccGgaagT_CaTCTcTAC | NNGG | 2 | 8 | SFB3 | YHR098C |
| | 300438: + | AGG-ATGG (SEQ ID | | | | | |
| | | NO: 70) | | | | | |
| gRNA6-3 | VIII: 397328- | aGtgTTgg_aTTCTATA | NGG | 2 | 7 | PEX28 | YHR150W |
| | 397351: + | CtGG-AGGC (SEQ ID | | | | | |
| | | NO: 71) | | | | | |
| gRNA6-3 | X: 36038- | cGacaggc_aTTCcATAC | NGG | 2 | 9 | OPT1 | YJL212C |
| | 36061: − | AGG-AGGA (SEQ ID | | | | | |
| | | NO: 72) | | | | | |
| gRNA6-3 | X: 53270- | ttGaaaca_CTTaaATACA | NAG | 2 | 9 | RCY1 | YJL204C |
| | 53293: + | GG-AAGA (SEQ ID | | | | | |
| | | NO: 73) | | | | | |
| gRNA6-3 | X: 98173- | aGcTgggT_CTTCTATA | NGG | 2 | 7 | CPS1 | YJL172W |
| | 98196: + | CAca-TGGG (SEQ ID | | | | | |
| | | NO: 74) | | | | | |
| gRNA6-3 | X: 146252- | tGcgTTgT_CTTtTATAt | NGG | 2 | 6 | SFH5 | YJL145W |
| | 146275: − | AGG-CGGA (SEQ ID | | | | | |
| | | NO: 75) | | | | | |
| giRNA6-3 | X: 544977- | cGacgaaa_CTcaTATAC | NGG | 2 | 9 | CDC8 | YJR057W |
| | 545000: − | AGG-AGGT (SEQ ID | | | | | |
| | | NO: 76) | | | | | |
| gRNA6-3 | X: 632972- | taaaaTTa_gTTCTATAa | NAG | 2 | 8 | CPA2 | YJR109C |
| | 632995: − | AGG-AAGA (SEQ ID | | | | | |
| | | NO: 77) | | | | | |
| gRNA6-3 | XI: 36621 - | tctaTagg_CTaCTATAC | NAG | 2 | 9 | SAC1 | YKL212W |
| | 36644: + | AtG-AAGG (SEQ ID | | | | | |
| | | NO: 78) | | | | | |
| gRNA6-3 | XI: 208050- | caaaaTaT_CTTtTATAC | NAG | 2 | 8 | YVK1 | YKL126W |
| | 208073: − | AaG-GAGA (SEQ ID | | | | | |
| | | NO: 79) | | | | | |
| gRNA6-3 | XI: 247133- | GtGgTcgc_CTTCTtTAC | NAG | 2 | 7 | LAP4 | YIKL103C |
| | 247156: − | AaG-AAGA (SEQ ID | | | | | |
| | | NO: 80) | | | | | |
| gRNA6-3 | XI: 304194- | aaGgTgca_CTTtTATAC | NNGG | 2 | 8 | | |
| | 304217: − | AaG-CTGG (SEQ ID | | | | | |
| | | NO: 81) | | | | | |
| gRNA6-3 | XI: 339579- | tatTTTTT_CTTCgATAt | NAG | 2 | 5 | MDM35 | YKL053C-A |
| | 339602: − | AGG-GAGA (SEQ ID | | | | | |
| | | NO: 82) | | | | | |
| gRNA6-3 | XI: 528075- | tGcTggag_CgTCTAcAC | NNGG | 2 | 8 | TRK2 | YKR050W |
| | 528098: + | AGG-GCGG (SEQ ID | | | | | |
| | | NO: 83) | | | | | |
| gRNA6-3 | XI: 582181- | ttccaTTT_CTTgTATAa | NAG | 2 | 7 | ECM4 | YKR076W |
| | 582204: + | AGG-TAGT (SEQ ID | | | | | |
| | | NO: 84) | | | | | |
| gRNA6-3 | XII: 248557- | atGTgcag_CTTCTAaAC | NNGG | 2 | 8 | YLR053C | YLR053C |
| | 248580: − | AGc-ACGG (SEQ ID | | | | | |
| | | NO: 85) | | | | | |
| gRNA6-3 | XII: 260469- | aaacggaT_CTTCTgTAC | NAG | 2 | 9 | REX2 | YLR059C |
| | 260492: + | AGc-GAGA (SEQ ID | | | | | |
| | | NO: 86) | | | | | |
| gRNA6-3 | XII: 322130- | tataTaTa_CaTaTATACA | NAG | 2 | 8 | XDJ1 | YLR090W |
| | 322153: − | GG-TAGG (SEQ ID NO: | | | | | |
| | | 87) | | | | | |
| gRNA6-3 | XII: 490870- | atGaTaaa_CTTCTAcAC | NAG | 2 | 8 | RRT15 | YLR162W-A |
| | 490893: + | tGG-AAGG (SEQ ID | | | | | |
| | | NO: 88) | | | | | |
| gRNA6-3 | XII: 531094- | GatcagTT_CTTtTATgC | NAG | 2 | 7 | ATG26 | YLR189C |
| | 531117: − | AGG-TAGA (SEQ ID | | | | | |
| | | NO: 89) | | | | | |
| gRNA6-3 | XII: 546408- | cttaggTc_CTTCTATtaA | NAG | 2 | 9 | NOP56 | YLR197W |
| | 546431: + | GG-AAGA (SEQ ID | | | | | |
| | | NO: 90) | | | | | |
| gRNA6-3 | XII: 708601- | aatTTcac_CTTCagTAC | NAG | 2 | 8 | NNT1 | YLR285W |
| | 708624: + | AGG-TAGA (SEQ ID | | | | | |
| | | NO: 91) | | | | | |
| gRNA6-3 | XII: 892092- | tGcTgTTT_CTTCTgTA | NGG | 2 | 5 | IKI3 | YLR384C |
| | 892115: − | gAGG-AGGT (SEQ ID | | | | | |
| | | NO: 92) | | | | | |
| gRNA6-3 | XIII: 150756- | cacaacaT_tTTtTATACA | NAG | 2 | 9 | PIF1 | YML061C |
| | 150779: − | GG-GAGT (SEQ ID NO: | | | | | |
| | | 93) | | | | | |
| gRNA6-3 | XIII: 559528- | cacaagTg_CTgCaATAC | NGG | 2 | 9 | YMR147W | YMR147W |
| | 559551: + | AGG-AGGA (SEQ ID | | | | | |
| | | NO: 94) | | | | | |
| gRNA6-3 | XIII: 647508- | tGcagaTT_CTTCTATgC | NAG | 2 | 7 | GYL1 | YMR192W |
| | 647531: − | AGt-CAGC (SEQ ID | | | | | |
| | | NO: 95) | | | | | |
| gRNA6-3 | XIII: 647736- | taaaggaT_CTTCTATAC | NNGG | 2 | 9 | GYL1 | YMR192W |
| | 647759: − | gGc-GTGG (SEQ ID | | | | | |
| | | NO: 96) | | | | | |
| gRNA6-3 | XIII: 804718- | GtagcTca_CcTCTATAC | NGG | 1 | 7 | PRP24 | YMR268C |
| | 804741: + | AGG-TGGT (SEQ ID | | | | | |
| | | NO: 97) | | | | | |
| gRNA6-3 | XIII: 919017- | cacggTcc_CTTCTATAa | NGG | 2 | 9 | SNO4 | YMR322C |
| | 919040: − | AGa-TGGT (SEQ ID | | | | | |
| | | NO: 98) | | | | | |
| gRNA6-3 | XIV: 30102- | GaGcaggg_CTTCTAaA | NNGG | 2 | 8 | FIG4 | YNL325C |
| | 30125: − | CAaG-ATGG (SEQ ID | | | | | |
| | | NO: 99) | | | | | |
| gRNA6-3 | XIV: 291671- | cataTTaT_CTcCTATAC | NGG | 2 | 7 | UBP10 | YNL186W |
| | 291694: + | AcG-AGGC (SEQ ID | | | | | |
| | | NO: 100) | | | | | |
| gRNA6-3 | XIV: 318849- | tcagaaTa_tTTCTATAtA | NAG | 2 | 9 | FMP41 | YNL168C |
| | 318872: + | GG-AAGT (SEQ ID NO: | | | | | |
| | | 101) | | | | | |
| gRNA6-3 | XIV: 476045- | acaaaTac_aTTaTATAC | NAG | 2 | 9 | PMS1 | YNL082W |
| | 476068: + | AGG-GAGT (SEQ ID | | | | | |
| | | NO: 102) | | | | | |
| gRNA6-3 | XIV: 504894- | atGggTTc_CTTCTtcAC | NAG | 2 | 7 | AQR1 | YNL065W |
| | 504917: + | AGG-TAGA (SEQ ID | | | | | |
| | | NO: 103) | | | | | |
| gRNA6-3 | XIV: 676546- | ttccgTTT_CTTCaATAg | NGG | 2 | 7 | CPR8 | YNR028W |
| | 676569: − | AGG-AGGA (SEQ ID | | | | | |
| | | NO: 104) | | | | | |
| gRNA6-3 | XIV: 775608- | aaccTTgT_CTTCaATcC | NGG | 2 | 7 | YNR073C | YNR073C |
| | 775631: + | AGG-CGGC (SEQ ID | | | | | |
| | | NO: 105) | | | | | |
| gRNA6-3 | XV: 9645- | aaGagTTc_tTTCTATAC | NAG | 2 | 7 | YOL163W | YOL163W |
| | 9668: + | AtG-TAGA (SEQ ID | | | | | |
| | | NO: 106) | | | | | |
| gRNA6-3 | XV: 124102- | tGaTcaaT_CTTCTAcgC | NAG | 1 | 7 | ITR2 | YOL103W |
| | 124125: + | AGG-GAGA (SEQ ID | | | | | |
| | | NO: 107) | | | | | |
| gRNA6-3 | XV: 251169- | tGtagcTT_CcTCTATAC | NNGG | 2 | 7 | NGL1 | YOL042W |
| | 251192: − | AtG-CTGG (SEQ ID | | | | | |
| | | NO: 108) | | | | | |
| gRNA6-3 | XV: 390765- | GtGTTgTT_CTTaTATA | NGG | 1 | 3 | HMS1 | YOR032C |
| | 390788: + | CAGG-AGGC (SEQ ID | | | | | |
| | | NO: 109) | | | | | |
| gRNA6-3 | XV: 787651- | attTcaca_CTTtTATACA | NGG | 2 | 9 | MET7 | YOR241W |
| | 787674: + | aG-AGGA (SEQ ID NO: | | | | | |
| | | 110) | | | | | |
| gRNA6-3 | XV: 1052491- | aacgaagT_CTTCTATAC | NAG | 2 | 9 | RDR1 | YOR380W |
| | 1052514: − | Aaa-GAGA (SEQ ID | | | | | |
| | | NO: 111) | | | | | |
| gRNA6-3 | XVI: 11926- | cacggTcc_CTTCTATAa | NGG | 2 | 9 | HSP32 | YPL280W |
| | 11949: + | AGa-TGGT (SEQ ID | | | | | |
| | | NO: 112) | | | | | |
| gRNA6-3 | XVI: 117820- | atcggTTT_CTTCTATtC | NAG | 2 | 7 | YPL229W | YPL229W |
| | 117843: + | AtG-TAGT (SEQ ID | | | | | |
| | | NO: 113) | | | | | |
| gRNA6-3 | XVI: 175377- | GaGcaacg_tTaCTATAC | NAG | 2 | 8 | OXR1 | YPL196W |
| | 175400: − | AGG-GAGT (SEQ ID | | | | | |
| | | NO: 114) | | | | | |
| gRNA6-3 | XVI: 415443- | ataTaTaT_tTTCTATAa | NAG | 2 | 7 | GCR1 | YPL075W |
| | 415466: − | AGG-TAGT (SEQ ID | | | | | |
| | | NO: 115) | | | | | |
| gRNA6-3 | XVI: 552238- | GccaTTgg_CTTCTAaA | NAG | 2 | 7 | ULA1 | YPL003W |
| | 552261: + | CAGc-TAGA (SEQ ID | | | | | |
| | | NO: 116) | | | | | |
| gRNA6-3 | XVI: 609128- | GtaaTaTg_CTTtTATAt | NNGG | 2 | 7 | EAF3 | YPR023C |
| | 609151: − | AGG-TTGG (SEQ ID | | | | | |
| | | NO: 117) | | | | | |
| gRNA6-3 | XVI: 771985- | tGcgcTaa_CTTCTATAa | NGG | 1 | 7 | CLB2 | YPR119W |
| | 772008: + | AGG-AGGG (SEQ ID | | | | | |
| | | NO: 118) | | | | | |
| gRNA9-1 | II: 124775- | aacacgcT_TTCCCTAGT | NAG | 1 | 8 | PIN4 | YBL051C |
| | 124798: + | CtG-TAGC (SEQ ID | | | | | |
| | | NO: 119) | | | | | |
| gRNA9-1 | II: 141015- | GccTAtgc_TTCaCTAG | NGG | 2 | 7 | PRE7 | YBL041W |
| | 141038: − | TCAc-AGGC (SEQ ID | | | | | |
| | | NO: 120) | | | | | |
| gRNA9-1 | II1: 190562- | tcActtcT_TTCCCTAcTC | NGG | 2 | 8 | PEP1 | YBL017C |
| | 190585: + | At-GGGC (SEQ ID NO: | | | | | |
| | | 121) | | | | | |
| gRNA9-1 | III: 174520- | agggcAaT_TTCCCcAaT | NNGG | 2 | 8 | SYP1 | YCR030C |
| | 174543: + | CAG-TTGG (SEQ ID | | | | | |
| | | NO: 122) | | | | | |
| gRNA9-1 | IV: 187150- | GTtTtcgT_TgCCCTcGT | NNGG | 2 | 6 | ATG9 | YDL149W |
| | 187173: + | CAG-CCGG (SEQ ID | | | | | |
| | | NO: 123) | | | | | |
| gRNA9-1 | IV: 203591- | tTgaccTT_TTCCtgAGT | NAG | 2 | 7 | BPL1 | YDL141W |
| | 203614: − | CAG-AAGA (SEQ ID | | | | | |
| | | NO: 124) | | | | | |
| gRNA9-1 | IV: 361768- | tgATAcag_TaCCCcAGT | NGG | 2 | 7 | MCH1 | YDL054C |
| | 361791: + | CAG-TGGC(SEQ ID | | | | | |
| | | NO: 125) | | | | | |
| gRNA9-1 | IV: 373436- | aatgAtaT_TTCCCcAtTC | NGG | 2 | 8 | FAD1 | YDL045C |
| | 373459: + | AG-TGGA (SEQ ID NO: | | | | | |
| | | 126) | | | | | |
| gRNA9-1 | IV: 512002- | tctccAgc_TTCaCTAGaC | NGG | 2 | 9 | LYS14 | YDR034C |
| | 512025: − | AG-TGGT (SEQ ID NO: | | | | | |
| | | 127) | | | | | |
| gRNA9-1 | IV: 1216099- | agcagcTg_TTtCaTAGT | NGG | 2 | 9 | XRS2 | YDR369C |
| | 1216122: + | CAG-CGGA (SEQ ID | | | | | |
| | | NO: 128) | | | | | |
| gRNA9-1 | IV: 1222881- | acAaAATc_TTCCCTA | NNGG | 2 | 6 | FRQ1 | YDR373W |
| | 1222904: − | GctAG-TTGG (SEQ ID | | | | | |
| | | NO: 129) | | | | | |
| gRNA9-1 | IV: 1453832- | aagTtgag_TTCtCaAGTC | NGG | 2 | 9 | SAM2 | YDR502C |
| | 1453855: + | AG-CGGT (SEQ ID NO: | | | | | |
| | | 130) | | | | | |
| gRNA9-1 | IX: 341811- | Gctatgca_TTCCCaAtTC | NAG | 2 | 9 | FAA3 | YIL009W |
| | 341834: − | AG-AAGA (SEQ ID | | | | | |
| | | NO: 131) | | | | | |
| gRNA9-1 | V: 77444- | tccTgcgT_TTCCgTAGT | NGG | 2 | 8 | YEF1 | YEL041W |
| | 77467: + | CAa-GGGT(SEQ ID | | | | | |
| | | NO: 132) | | | | | |
| gRNA9-1 | V: 339086- | catctggT_TTCtCaAGTC | NGG | 2 | 9 | TRP2 | YER090W |
| | 339109: + | AG-CGGT (SEQ ID NO: | | | | | |
| | | 133) | | | | | |
| gRNA9-1 | V: 394153- | aaAggtga_TTCtCTAGT | NNGG | 2 | 9 | BOI2 | YER114C |
| | 394176: − | CAc-GTGG (SEQ ID | | | | | |
| | | NO: 134) | | | | | |
| gRNA9-1 | VI: 190133- | catTttaT_TTCtaTAGTC | NAG | 2 | 8 | FAB1 | YFR019W |
| | 190156: − | AG-AAGT (SEQ ID NO: | | | | | |
| | | 135) | | | | | |
| gRNA9-1 | VI: 191084- | aTggAtTa_TTCCtTAGT | NGG | 2 | 7 | FAB1 | YFR019W |
| | 191107: + | CAt-TGGT (SEQ ID | | | | | |
| | | NO: 136) | | | | | |
| gRNA9-1 | VIII: 89453- | cacattTg_TTCCaTtGTC | NNGG | 2 | 9 | YHL009W-B | YHL009W-B |
| | 89476: − | AG-TTGG (SEQ ID NO: | | | | | |
| | | 137) | | | | | |
| gRNA9-1 | VIII: 239668- | ccAaAtTT_TTCCCcAG | NGG | 2 | 6 | YHR071C-A | YHR071C-A |
| | 239691: − | TgAG-GGGA (SEQ ID | | | | | |
| | | NO: 138) | | | | | |
| gRNA9-1 | VIII: 294172- | tcAgttcT_TTCCCTAGT | NAG | 2 | 8 | HXT5 | YHR096C |
| | 294195: + | atG-TAGT (SEQ ID NO: | | | | | |
| | | 139) | | | | | |
| gRNA9-1 | X: 201460- | cacattTg_TTCCaTtGTC | NNGG | 2 | 9 | YJL113W | YJL113W |
| | 201483: − | AG-TTGG (SEQ ID NO: | | | | | |
| | | 140) | | | | | |
| gRNA9-1 | X: 361346- | actcggaT_TTCCCTgGT | NAG | 2 | 9 | YJL043W | YJL043W |
| | 361369: − | CtG-GAGC (SEQ ID | | | | | |
| | | NO: 141) | | | | | |
| gRNA9-1 | X: 377567- | tagTAATa_TTtaCTAGT | NGG | 2 | 6 | IRC18 | YJL037W |
| | 377590: − | CAG-TGGG (SEQ ID | | | | | |
| | | NO: 142) | | | | | |
| gRNA9-1 | X: 389949- | tctTtgaa_TTCCCTttTCA | NAG | 2 | 9 | VPS53 | YJL029C |
| | 389972: + | G-AAGT (SEQ ID NO: | | | | | |
| | | 143) | | | | | |
| gRNA9-1 | X: 716216- | GatcAcTT_TTtCCcAGT | NAG | 2 | 6 | DAN4 | YJR151C |
| | 716239: + | CAG-TAGA (SEQ ID | | | | | |
| | | NO: 144) | | | | | |
| gRNA9-1 | XI: 101766- | GTtgAAaT_TTCtCTAG | NGG | 2 | 5 | FAS1 | YKL182W |
| | 101789: + | TcAa-TGGT (SEQ ID | | | | | |
| | | NO: 145) | | | | | |
| gRNA9-1 | XI: 290574- | cTtgACTT_TTCCCTAG | NAG | 2 | 6 | DHR2 | YKL078W |
| | 290597: − | TtcG-TAGA (SEQ ID | | | | | |
| | | NO: 146) | | | | | |
| gRNA9-1 | XI: 451589- | aacTcTg_TTCCCTgGc | NAG | 2 | 8 | MRPL13 | YKR006C |
| | 451612: − | CAG-CAGT (SEQ ID | | | | | |
| | | NO: 147) | | | | | |
| gRNA9-1 | XI: 609043- | GTAatccg_TTCagTAGT | NGG | 2 | 7 | PXL1 | YKR090W |
| | 609066: + | CAG-AGGA (SEQ ID | | | | | |
| | | NO: 148) | | | | | |
| gRNA9-1 | XII: 329488- | tgccgtcT_gaCCCTAGTC | NAG | 2 | 9 | GIS3 | YLR094C |
| | 329511: + | AG-GAGC (SEQ ID | | | | | |
| | | NO: 149) | | | | | |
| gRNA9-1 | XII: 689622- | ccAgtgcT_TTCCCTAG | NGG | 1 | 7 | PIG1 | YLR273C |
| | 689645: + | TCcG-TGGT (SEQ ID | | | | | |
| | | NO: 150) | | | | | |
| gRNA9-1 | XII: 780325- | aTATAtaa_aTCCCTcGT | NGG | 2 | 6 | PEX30 | YLR324W |
| | 780348: − | CAG-GGGA (SEQ ID | | | | | |
| | | NO: 151) | | | | | |
| gRNA9-1 | XII: 820724- | aaATAAaT_TgCCCgAG | NGG | 2 | 5 | YLR345W | YLR345W |
| | 820747: − | TCAG-TGGA (SEQ ID | | | | | |
| | | NO: 152) | | | | | |
| gRNA9-1 | XIII: 471225- | agtgtATT_TTCCCTccT | NGG | 2 | 7 | YMR102C | YMR102C |
| | 471248: + | CAG-GGGA (SEQ ID | | | | | |
| | | NO: 153) | | | | | |
| gRNA9-1 | XIII: 794455- | GagggAga_TgCCCTgG | NAG | 2 | 8 | YMR262W | YMR262W |
| | 794478: + | TCAG-GAGC (SEQ ID | | | | | |
| | | NO: 154) | | | | | |
| gRNA9-1 | XIV: 48908- | aaATgtca_TTCCaTAGc | NGG | 2 | 8 | RFA2 | YNL312W |
| | 48931: + | CAG-TGGA (SEQ ID | | | | | |
| | | NO: 155) | | | | | |
| gRNA9-1 | XIV: 176427- | cTtTctaa_TTCCCTcaTC | NNGG | 2 | 8 | RAD50 | YNL250W |
| | 176450: + | AG-GCGG (SEQ ID | | | | | |
| | | NO: 156) | | | | | |
| gRNA9-1 | XIV: 197156- | accggtcT_TTCCaTAGT | NAG | 2 | 9 | ZWF1 | YNL241C |
| | 197179: + | CAa-GAGA (SEQ ID | | | | | |
| | | NO: 157) | | | | | |
| gRNA9-1 | XIV: 660306- | cgActAgT_TTCCCcAG | NNGG | 2 | 7 | ACC1 | YNR016C |
| | 660329: + | TCtG-ACGG (SEQ ID | | | | | |
| | | NO: 158) | | | | | |
| gRNA9-1 | XV: 660238- | tTcTcATT_TTtCCTAtT | NGG | 2 | 5 | ALE1 | YOR175C |
| | 660261: − | CAG-AGGA (SEQ ID | | | | | |
| | | NO: 159) | | | | | |
| gRNA9-1 | XV: 729770- | tccaAgcT_TTCtCTtGTC | NNGG | 2 | 8 | NOC2 | YOR206W |
| | 729793: + | AG-CTGG (SEQ ID NO: | | | | | |
| | | 160) | | | | | |
| gRNA9-1 | XVI: 82193- | GTcagATg_TgCCCTAG | NGG | 1 | 5 | GAL4 | YPL248C |
| | 82216: + | TCAG-CGGA (SEQ ID | | | | | |
| | | NO: 161) | | | | | |
| gRNA9-1 | XVI: 142896- | tTgattcg_gTCCCTcGTC | NAG | 2 | 9 | BMS1 | YPL217C |
| | 142919: − | AG-GAGA (SEQ ID | | | | | |
| | | NO: 162) | | | | | |
| gRNA9-1 | XVI: 237718- | tccTgtcT_TTCCgTgGTC | NGG | 2 | 8 | ATG29 | YPL166W |
| | 237741: − | AG-TGGG (SEQ ID NO: | | | | | |
| | | 163) | | | | | |
| gRNA9-1 | XVI: 337889- | tcATtcTa_TTCCtTtGTC | NAG | 2 | 7 | PEX25 | YPL112C |
| | 337912: − | AG-TAGA (SEQ ID NO: | | | | | |
| | | 164) | | | | | |
| gRNA9-1 | XVI: 439175- | cacattTg_TTCCaTtGTC | NNGG | 2 | 9 | YPL060C-A | YPL060C-A |
| | 439198: + | AG-TTGG (SEQ ID NO: | | | | | |
| | | 165) | | | | | |
| gRNA9-1 | XVI: 544973- | GacaAAcc_TTCCtTgGT | NAG | 2 | 7 | NCR1 | YPL006W |
| | 544996: − | CAG-CAGC (SEQ ID | | | | | |
| | | NO: 166) | | | | | |
| gRNA9-1 | XVI: 587480- | GTActcTa_cTCCCaAG | NGG | 2 | 6 | YPR014C | YPR014C |
| | 587503: + | TCAG-CGGA (SEQ ID | | | | | |
| | | NO: 167) | | | | | |
| gRNA9-1 | XVI: 619512- | aTtggcTc_TTCtCTcGTC | NAG | 2 | 8 | ATH1 | YPR026W |
| | 619535: + | AG-TAGG (SEQ ID NO: | | | | | |
| | | 168) | | | | | |
| gRNA9-1 | XVI: 883548- | tTccAAgT_TTaCCTAG | NAG | 2 | 6 | YPR170C | YPR170C |
| | 883571: + | cCAG-AAGA (SEQ ID | | | | | |
| | | NO: 169) | | | | | |

*In the Target Sequence, the first dash is used to separate the non-seed (first 8 nucleotides) and seed sequences (the next 12 nucleotides); the second dash is used to separate the gRNA sequences (non-seed and seed) with PAM domain sequences (indicated in the 4th column). Capital nucleotides are matched to the gRNA sequences, and vice versa.

Transcriptional profile of S. cerevisiae screen cells expressing gRNA 9-1 and dCas9-VP64 was compared to cells expressing dCas9-VP64 but no gRNA using RNA-sequencing to map transcriptional perturbations enacted by the αSyn-protective crisprTF (FIG. 1C). 114 genes were identified as differentially expressed with at least two-fold changes in mRNA expression levels compared with the no-gRNA control (FDR-adjusted p-value≤0.1) (Table 1 and summarized in Table 3). The majority of these genes (93%) have not been previously identified in single gene knockout and over-expression screens as suppressors of αSyn toxicity (54, 55). Interestingly, the genes identified as being modulated by gRNA 9-1 were enriched in Gene Ontology (GO) categories including protein quality control, ER/Golgi trafficking, lipid metabolism, mitochondrial function, and stress responses (Table 4). Almost all of the newly identified genes only exhibited modest changes in gene expression (109 out of 114 genes had fold-changes<5).

TABLE 3
Summary of top-ranked genes that were found to be differentially regulated
by gRNA 9-1 and suppressed αSyn toxicity in yeast when overexpressed. *

| | Human | Log₂(fold | Survival | Fluorescent | Biological |
|---|---|---|---|---|---|
| Yeast Gene | Homologs | change) | Score | Foci Score | Function |
| SNO4/HSP34 | PARK7 | 2.035 | 4.5 | 3 | Chaperone and |
| | | | | | cysteine protease |
| HSP32 | PARK7 | −9.593 | 4.5 | 3.5 | Chaperone and |
| | | | | | cysteine protease |
| HSP42 | HSPB1, HSPB3, | 1.434 | 4 | 2.5 | Chaperone |
| | HSPB6, HSPB7, | | | | |
| | HSPB8, HSPB9 | | | | |
| SIS1 | DNAJB1-B9 | 1.154 | 4.5 | 1.5 | Chaperone |
| GGA1 | GGA1, GGA2, | 1.241 | 4.5 | 3 | ER to Golgi |
| | GGA3 | | | | vesicular |
| | | | | | trafficking |
| SRN2 | | 1.031 | 4.5 | 2.5 | Ubiquitin- |
| | | | | | dependent protein |
| | | | | | sorting |
| SAF1 | ALS2, RCC1 | 1.18 | 4.5 | 2 | Proteasome- |
| | | | | | dependent |
| | | | | | degradation |
| TRX1 | TXN, TXNDC2, | 1.072 | 4.5 | 2.5 | Thioredoxin |
| | TXNDC8 | | | | |
| TIM9 | TIMM9 | 3.846 | 4.5 | 2.5 | Mitochondrial |
| | | | | | intermembrane |
| | | | | | protein |
| OXR1 | OXR1, NCOA7, | 1.003 | 4 | 2.5 | Oxidative damage |
| | TLDC2 | | | | resistance |
| STF2 | SERBP1, HABP4 | 2.004 | 4.5 | 2.5 | mRNA |
| | | | | | stablization |
| gRNA 9-1 | | | 5 | 5 | |
| UBP3 | | | 4.5 | 3.5 | |
| Vector | | | 1 | 1 | |

* A complete list of the genes found to be differentially modulated by gRNA 9-1 is provided in Table 1.

TABLE 4
Functional categories of genes regulated by gRNA 9-1

| | | |
|---|---|---|
| Category | p-value | In Category from Cluster |

MIPS Functional Classification

| | | |
|---|---|---|
| enzyme inhibitor | 0.000537 | YPI1 SPL2 VHS3 |
| [18.02.01.02] | | |
| unfolded protein response | 0.001097 | HSP42 HSP78 TIM9 SNO4 SIS1 HSP32 |
| (e.g. ER quality control) | | |
| [32.01.07] | | |
| UNCLASSIFIED | 0.002005 | YBR126W-A RTC2 YBR230W-A SAF1 |
| PROTEINS [99] | | YBR285W COS2 TMA17 TVP15 ARP10 |
| | | YDR169C-A YER053C-A YER121W |
| | | YFL012W KEG1 YGL101W YGL258W- |
| | | A YGR130C AIM17 YHR086W-A RTC3 |
| | | ANS1 FMP33 YJL163C YJR005C-A |
| | | YKL100C YLR149C YLR257W |
| | | YMR247W-A BXI1 OPI10 RRT8 PHM7 |
| | | YOL114C YOL164W-A PNS1 YOR292C |
| | | YPL247C |
| regulation of phosphate | 0.003302 | GIP2 YPI1 VHS3 |
| metabolism [01.04.04] | | |
| ribosome biogenesis [12.01] | 0.004589 | ARX1 RIX1 ECM16 RRP12 NOG1 |
| stress response [32.01] | 0.00638 | HSP30 TPS2 CYC7 STF2 XBP1 |
| | | YJL144W PAU20 VHS3 |
| PROTEIN FATE (folding, | 0.007599 | SNO4 HSP32 |
| modification, destination) | | |
| [14] | | |
| rRNA processing [11.04.01] | 0.008172 | UTP8 RIX1 UTP10 ERB1 ECM16 HAS1 |
| | | DBP2 RRP12 |
| homeostasis of phosphate | 0.009662 | PHO89 PHO84 |
| [34.01.03.03] | | |

GO Molecular Function

| | | |
|---|---|---|
| molecular_function | 6.14E−08 | FRT2 YBL086C YBR056W RTC2 |
| [GO:0003674] | | YBR230W-A YBR238C YBR285W COS2 |
| | | HSP30 ERP3 TMA17 TVP15 ARX1 |
| | | YDR169C-A EMI2 YER053C-A PET117 |
| | | YER121W YFL012W KEG1 YGL101W |
| | | YGL258W-A STF2 YGR130C FHN1 |
| | | BNS1 AIM17 YHR086W-A RTC3 ANS1 |
| | | RIX1 YJL144W FMP33 YJL163C |
| | | YJR005C-A YKL100C YLR149C |
| | | YLR164W YLR257W ECM19 SUR7 |
| | | COS3 ERB1 YMR244W YMR247W-A |
| | | YMR262W MDG1 BXI1 OPI10 RRT8 |
| | | PHM7 YOL114C PAU20 YOL164W-A |
| | | PNS1 FSH3 YOR292C RRP12 OXR1 |
| | | YPL247C SUE1 |
| transporter activity | 0.000827416 | GIT1 YDL199C HXT7 HXT3 MCH2 |
| [GO:0005215] | | AQY2 PHO84 |
| protein phosphatase inhibitor | 0.00170312 | YPI1 VHS3 |
| activity [GO:0004864] | | |
| inorganic phosphate | 0.00280679 | PHO89 PHO84 |
| transmembrane transporter | | |
| activity [GO:0005315] | | |
| symporter activity | 0.00759902 | PHO89 MCH2 |
| [GO:0015293] | | |

GO Cellular Component

| | | |
|---|---|---|
| membrane raft [GO:0045121] | 0.000258487 | YGR130C FHN1 SUR7 MDG1 |
| 90S preribosome | 0.00158222 | PRP43 UTP8 UTP10 ECM16 HAS1 |
| [GO:0030686] | | RRP12 |
| plasma membrane | 0.00244257 | PHO89 GEX1 HSP30 GIT1 HXT7 HXT3 |
| [GO:0005886] | | FHN1 ANS1 AQY2 SUR7 MDG1 ATO2 |
| | | PHM7 PNS1 |
| integral to membrane | 0.00277969 | FRT2 RTC2 PHO89 COS2 GEX1 HSP30 |
| [GO:0016021] | | GIT1 ERP3 YDL199C TVP15 HXT7 |
| | | HXT3 TIM9 YER053C-A KEG1 FHN1 |
| | | UTP10 FMP33 YJL163C YKL100C |
| | | MCH2 GPT2 YLR164W ECM19 SUR7 |
| | | PHO84 COS3 YMR244W BXI1 ATO2 |
| | | RRT8 PHM7 PAU20 PNS1 YOR292C |
| t-UTP complex | 0.00576345 | UTP8 UTP10 |
| [GO:0034455] | | |
| fungal-type vacuole | 0.00584756 | COS2 TRX1 COS3 BXI1 PHM7 |
| [GO:0000324] | | YOR292C |
| membrane [GO:0016020] | 0.00591148 | FRT2 RTC2 YBR238C PHO89 COS2 |
| | | GEX1 HSP30 GIT1 ERP3 YDL199C |
| | | TVP15 HXT7 HXT3 TIM9 YER053C-A |
| | | KEG1 FHN1 ANS1 ATG7 FMP33 |
| | | YJL163C YKL100C MCH2 GPT2 AQY2 |
| | | TRX1 SRN2 YLR164W ECM19 SUR7 |
| | | PHO84 COS3 YMR244W MDG1 BXI1 |
| | | ATO2 RRT8 PHM7 PAU20 PNS1 |
| | | YOR292C |
| cellular_component | 0.00757535 | YBL086C YBR230W-A YBR285W |
| [GO:0005575] | | YDR169C-A YER121W YFL012W |
| | | YGL258W-A BNS1 YHR086W-A ANS1 |
| | | YJR005C-A YLR149C YMR244W |
| | | YMR247W-A YMR262W SNO4 |
| | | YOL114C PAU20 YOL164W-A FSH3 |
| | | HSP32 |
| rDNA heterochromatin | 0.00759902 | UTP8 UTP10 |
| [GO:0033553] | | |
| eisosome [GO:0032126] | 0.00966151 | YGR130C SUR7 |

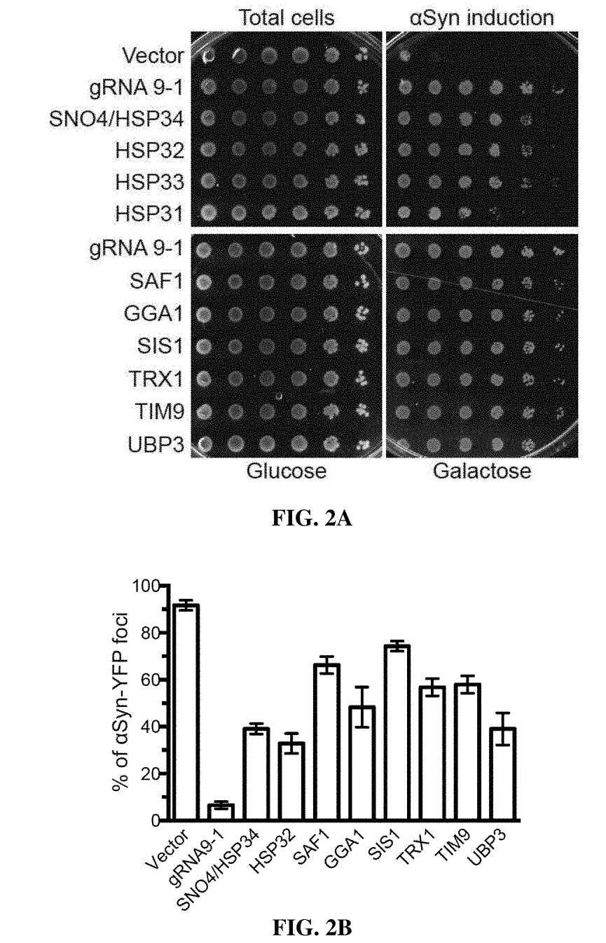

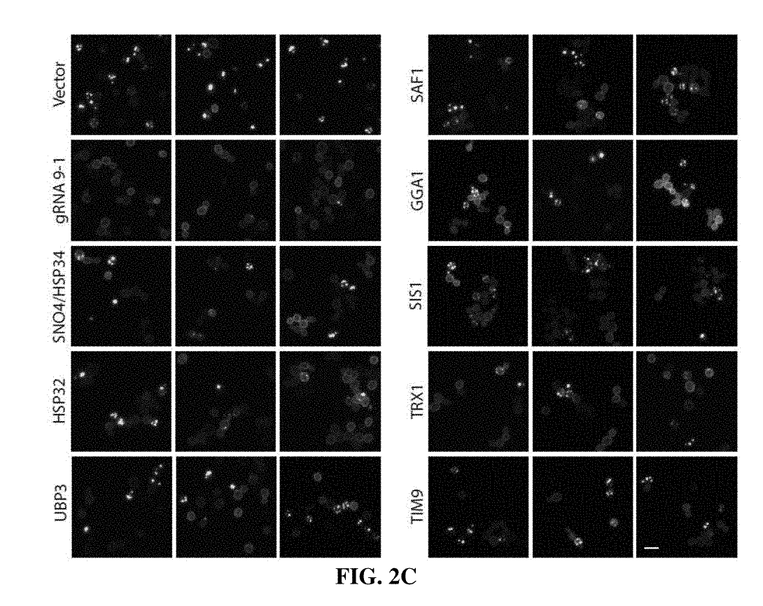

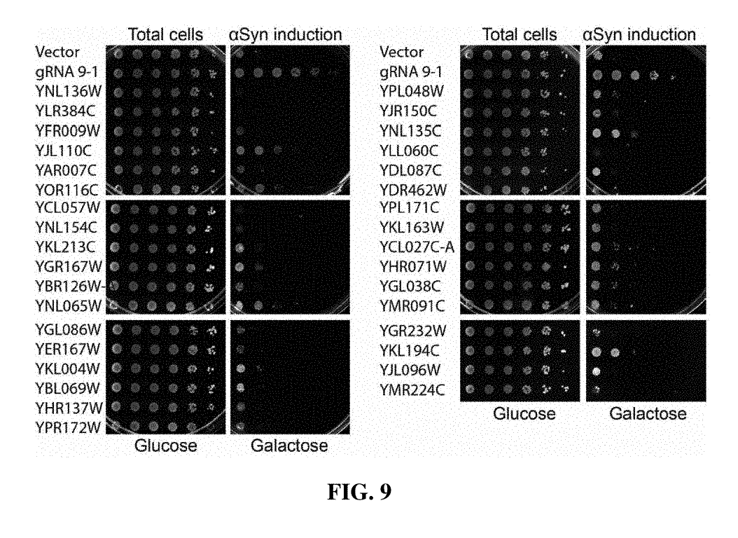

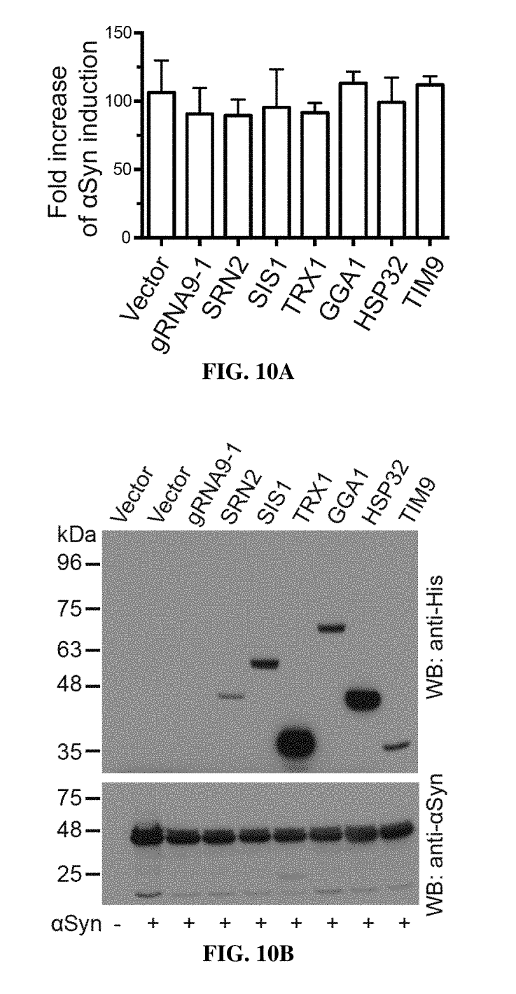

The genes identified as differentially expressed in cells expressing the gRNA 9-1 were systematically tested for their ability to suppress αSyn toxicity in the screen strain. It was found that over-expression of 57 out of 94 (60.4%) genes significantly suppressed αSyn toxicity (FIG. 8. Table 1 and summarized in Table 3, and representative candidates are shown in FIG. 2A). In contrast, only 5 out of 34 (14.7%) genes randomly chosen from the yeast ORF library were able to suppress αSyn toxicity when over-expressed (FIG. 9; Table 5). There was no significant correlation between observed αSyn expression levels and toxicity (FIGS. 10A and 10B). UBP3 (ubiquitin-specific protease), which was previously shown to be a strong suppressor of αSyn toxicity and known to participate in the degradation of misfolded proteins in the vesicular trafficking processes, was used as a positive control (44, 45, 54). It was found that 29 genes, which were identified as being modulated by gRNA 9-1, exhibited αSyn-toxicity protection levels similar to or better (more protective) than UBP3. Notably, gRNA 9-1 alone out-performed (was more protective) the over-expression of any single genes in suppressing αSyn toxicity (based on cell viability assay results shown in FIGS. 2A-2C), suggesting that gRNA 9-1 plays a master role in regulating multiple genes to mitigate αSyn stress.

TABLE 5
List of genes randomly chosen from yeast ORF library
and the αSyn suppressive effects when overexpressed

| | | αSyn | |
|---|---|---|---|
| Systematic | Standard | Suppression | |
| Name | Name | Score | Description |
| YNL136W | EAF7 | 0 | Subunit of the NuA4 histone acetyltransferase |
| | | | complex; NuA4 acetylates the N-terminal tails of |
| | | | histories H4 and H2A |
| YLR384C | IKI3 | 0 | Subunit of Elongator complex; Elongator is |
| | | | required for modification of wobble nucleosides in |
| | | | tRNA; maintains structural integrity of Elongator; |
| | | | homolog of human IKAP, mutations in which cause |
| | | | familial dysautonomia (FD) |
| YFR009W | GCN20 | 1 | Positive regulator of the Gcn2p kinase activity; |
| | | | forms a complex with Gcn1p; proposed to stimulate |
| | | | Gcn2p activation by an uncharged tRNA |
| YJL110C | GZF3 | 2.5 | GATA zinc finger protein; negatively regulates |
| | | | nitrogen catabolic gene expression by competing |
| | | | with Gat1p for GATA site binding; function |
| | | | requires a repressive carbon source; dimerizes with |
| | | | Dal80p and binds to Tor1p; GZF3 has a paralog, |
| | | | DAL80, that arose from the whole genome |
| | | | duplication |
| YAR007C | RFA1 | 1 | Subunit of heterotrimeric Replication Protein A |
| | | | (RPA); RPA is a highly conserved single-stranded |
| | | | DNA binding protein involved in DNA replication, |
| | | | repair, and recombination; RPA protects against |
| | | | inappropriate telomere recombination, and upon |
| | | | telomere uncapping, prevents cell proliferation by a |
| | | | checkpoint-independent pathway; role in DNA |
| | | | catenation/decatenation pathway of chromosome |
| | | | disentangling; relocalizes to the cytosol in response |
| | | | to hypoxia |
| YOR116C | RPO31 | 2* | RNA polymerase III largest subunit C160; part of |
| | | | core enzyme; similar to bacterial beta-prime subunit |
| | | | and to RPA190 and RPO21 |
| YCL057W | PRD1 | 1 | Zinc metalloendopeptidase; found in the cytoplasm |
| | | | and intermembrane space of mitochondria; with |
| | | | Cym1p, involved in degradation of mitochondrial |
| | | | proteins and of presequence peptides cleaved from |
| | | | imported proteins; protein abundance increases in |
| | | | response to DNA replication stress |
| YNL154C | YCK2 | 0 | Palmitoylated plasma membrane-bound casein |
| | | | kinase I (CK1) isoform; shares redundant functions |
| | | | with Yck1p in morphogenesis, proper septin |
| | | | assembly, endocytic trafficking, and glucose |
| | | | sensing; stabilized by Sod1p binding in the |
| | | | presence of glucose and oxygen, causing glucose |
| | | | repression of respiratory metabolism; YCK2 has a |
| | | | paralog, YCK1, that arose from the whole genome |
| | | | duplication |
| YKL213C | DOA1 | 0 | WD repeat protein required for ubiquitin-mediated |
| | | | protein degradation; forms a complex with Cdc48p; |
| | | | plays a role in controlling cellular ubiquitin |
| | | | concentration; also promotes efficient NHEJ in |
| | | | postdiauxic/stationary phase; facilitates N- |
| | | | terminus-dependent proteolysis of centromeric |
| | | | histone H3 (Cse4p) for faithful chromosome |
| | | | segregation; protein increases in abundance and |
| | | | relocalizes from nucleus to nuclear periphery upon |
| | | | DNA replication stress |
| YGR167W | CLC1 | 0 | Clathrin light chain; subunit of the major coat |
| | | | protein involved in intracellular protein transport |
| | | | and endocytosis; regulates endocytic progression; |
| | | | thought to regulate clathrin function; the clathrin |
| | | | triskelion is a trimeric molecule composed of three |
| | | | heavy chains that radiate from a vertex and three |
| | | | light chains which bind noncovalently near the |
| | | | vertex of the triskelion |
| YBR126W- | | 1 | Protein of unknown function; identified by gene- |
| A | | | trapping, microarray analysis, and genome-wide |
| | | | homology searches; mRNA identified as translated |
| | | | by ribosome profiling data; partially overlaps the |
| | | | dubious ORF YBR126W-B |
| YNL065W | AQR1 | 2 | Plasma membrane transporter of the major |
| | | | facilitator superfamily; member of the 12-spanner |
| | | | drug: H(+) antiporter DHA1 family; confers |
| | | | resistance to short-chain monocarboxylic acids and |
| | | | quinidine; involved in the excretion of excess |
| | | | amino acids; AQR1 has a paralog, QDR1, that |
| | | | arose from the whole genome duplication; |
| | | | relocalizes from plasma membrane to cytoplasm |
| | | | upon DNA replication stress |
| YGL086W | MAD1 | 1 | Coiled-coil protein involved in spindle-assembly |
| | | | checkpoint; required for inhibition of |
| | | | karyopherin/importin Pse1p (aka Kap121p) upon |
| | | | spindle assembly checkpoint arrest; phosphorylated |
| | | | by Mps1p upon checkpoint activation which leads |
| | | | to inhibition of anaphase promoting complex |
| | | | activity; forms a complex with Mad2p; gene dosage |
| | | | imbalance between MAD1 and MAD2 leads to |
| | | | chromosome instability |
| YER167W | BCK2 | 1 | Serine/threonine-rich protein involved in PKC1 |
| | | | signaling pathway; protein kinase C (PKC1) |
| | | | signaling pathway controls cell integrity; |
| | | | overproduction suppresses pkc1 mutations |
| YKL004W | AUR1 | 1 | Phosphatidylinositol:ceramide phosphoinositol |
| | | | transferase; required for sphingolipid synthesis; can |
| | | | mutate to confer aureobasidin A resistance; also |
| | | | known as IPC synthase |
| YBL069W | AST1 | 1 | Lipid raft associated protein; interacts with the |
| | | | plasma membrane ATPase Pma1p and has a role in |
| | | | its targeting to the plasma membrane by influencing |
| | | | its incorporation into lipid rafts; sometimes |
| | | | classified in the medium-chain |
| | | | dehydrogenase/reductases (MDRs) superfamily; |
| | | | AST1 has a paralog, AST2, that arose from the |
| | | | whole genome duplication |
| YHR137W | ARO9 | 1 | Aromatic aminotransferase II; catalyzes the first |
| | | | step of tryptophan, phenylalanine, and tyrosine |
| | | | catabolism |
| YPR172W | | 1 | Protein of unknown function; predicted to encode a |
| | | | pyridoxal 5′-phosphate synthase based on sequence |
| | | | similarity but purified protein does not possess this |
| | | | activity, nor does it bind flavin mononucleotide |
| | | | (FMN); transcriptionally activated by Yrm1p along |
| | | | with genes involved in multidrug resistance; |
| | | | YPR172W has a paralog, YLR456W, that arose |
| | | | from the whole genome duplication |
| YPL048W | CAM1 | 1 | One of two isoforms of the gamma subunit of |
| | | | eEF1B; stimulates the release of GDP from eEF1A |
| | | | (Tef1p/Tef2p) post association with the ribosomal |
| | | | complex with eEF1Balpha subunit; nuclear protein |
| | | | required for transcription of MXR1; binds the |
| | | | MXR1 promoter in the presence of other nuclear |
| | | | factors; binds calcium and phospholipids |
| YJR150C | DAN1 | 1 | Cell wall mannoprotein; has similarity to Tir1p, |
| | | | Tir2p, Tir3p, and Tir4p; expressed under anaerobic |
| | | | conditions, completely repressed during aerobic |
| | | | growth |
| YNL135C | FPR1 | 2.5 | Peptidyl-prolyl cis-trans isomerase (PPIase); binds |
| | | | to the drugs FK506 and rapamycin; also binds to |
| | | | the nonhistone chromatin binding protein Hmo1p |
| | | | and may regulate its assembly or function; N- |
| | | | terminally propionylated in vivo; mutation is |
| | | | functionally complemented by human FKBP1A |
| YLL060C | GTT2 | 0 | Glutathione S-transferase capable of |
| | | | homodimerization; functional overlap with Gtt2p, |
| | | | Grx1p, and Grx2p; protein abundance increases in |
| | | | response to DNA replication stress |
| YDL087C | LUC7 | 1 | Essential protein associated with the U1 snRNP |
| | | | complex; splicing factor involved in recognition of |
| | | | 5′ splice site; contains two zinc finger motifs; N- |
| | | | terminal zinc finger binds pre-mRNA; relocalizes to |
| | | | the cytosol in response to hypoxia |
| YDR462W | MRPL28 | 1 | Mitochondrial ribosomal protein of the large |
| | | | subunit; protein abundance increases in response to |
| | | | DNA replication stress |
| YPL171C | OYE3 | 1 | Conserved NADPH oxidoreductase containing |
| | | | flavin mononucleotide (FMN); homologous to |
| | | | Oye2p with different ligand binding and catalytic |
| | | | properties; has potential roles in oxidative stress |
| | | | response and programmed cell death |
| YKL163W | PIR3 | 1 | O-glycosylated covalently-bound cell wall protein; |
| | | | required for cell wall stability; expression is cell |
| | | | cycle regulated, peaking in M/G1 and also subject |
| | | | to regulation by the cell integrity pathway; coding |
| | | | sequence contains length polymorphisms in |
| | | | different strains; PIR3 has a paralog, HSP150, that |
| | | | arose from the whole genome duplication |
| YCL027C-A | HBN1 | 1 | Protein of unknown function; similar to bacterial |
| | | | nitroreductases; green fluorescent protein (GFP)- |
| | | | fusion protein localizes to the cytoplasm and |
| | | | nucleus; protein becomes insoluble upon |
| | | | intracellular iron depletion; protein abundance |
| | | | increases in response to DNA replication stress |
| YHR071W | PCL5 | 1 | Cyclin: interacts with and phosphorylated by |
| | | | Pho85p cyclin-dependent kinase (Cdk), induced by |
| | | | Gcn4p at level of transcription, specifically |
| | | | required for Gcn4p degradation, may be sensor of |
| | | | cellular protein biosynthetic capacity |
| YGL038C | OCH1 | 1 | Mannosyltransferase of the cis-Golgi apparatus; |
| | | | initiates the polymannose outer chain elongation of |
| | | | N-linked oligosaccharides of glycoproteins |
| YMR091C | NPL6 | 1 | Component of the RSC chromatin remodeling |
| | | | complex; interacts with Rsc3p, Rsc30p, Ldb7p, and |
| | | | Htl1p to form a module important for a broad range |
| | | | of RSC functions |
| YGR232W | NAS6 | 1 | Assembly chaperone for the 19S proteasome |
| | | | regulatory particle base; proteasome-interacting |
| | | | protein involved in the assembly of the base |
| | | | subcomplex of the 19S proteasomal regulatory |
| | | | particle (RP); ortholog of human oncoprotein |
| | | | gankyrin, which interacts with the Rb tumor |
| | | | suppressor and CDK4/6 |
| YKL194C | MST1 | 2 | Mitochondrial threonyl-tRNA synthetase; |
| | | | aminoacylates both the canonical threonine tRNA |
| | | | tT(UGU)Q1 and the unusual threonine tRNA |
| | | | tT(UAG)Q2 in vitro; lacks a typical editing domain, |
| | | | but has pre-transfer editing activity stimulated by |
| | | | the unusual tRNA-Thr |
| YJL096W | MRPL49 | 1 | Mitochondrial ribosomal protein of the large |
| | | | subunit |
| YMR224C | MRE11 | 1 | Nuclease subunit of the MRX complex with |
| | | | Rad50p and Xrs2p; complex functions in repair of |
| | | | DNA double-strand breaks and in telomere |
| | | | stability; Mre11p associates with Ser/Thr-rich |
| | | | ORFs in premeiotic phase; nuclease activity |
| | | | required for MRX function; widely conserved; |
| | | | forms nuclear foci upon DNA replication stress |

Alterations in membrane trafficking and localization of αSyn from the plasma membrane into cytoplasmic foci are well-established hallmarks of PD (56). Owing to highly conserved mechanisms involved in membrane trafficking, yeast cells have been used to study αSyn-coupled vesicular trafficking defects, which has led to mechanistic insights into modifiers of αSyn toxicity, such as UBP3 and the Rab family GTPase YPT1 and their human homolog counterparts (44, 45, 54). The effect of gRNA 9-1 on the localization of αSyn-YFP was assessed by microscopy. In this assay, aggregated αSyn-YFP can be detected as cytoplasmic foci, which are distinguishable from the membrane-localized, non-aggregated form of the protein. As shown in FIGS. 2B and 2C, upon 6 hours of αSyn induction, 92% of yeast cells with dCas9-VP64 but no gRNA (negative control) contained aggregated αSyn-YFP foci. Over-expression of dCas9-VP64 along with gRNA 9-1 resulted in localization of αSyn-YFP to the plasma membrane such that αSyn-YFP foci were observed in only ˜7% of cells. This was significantly lower than cells overexpressing UBP3 (˜39% cells with αSyn-YFP foci), which was used as a positive control.

Interestingly, one of the functional categories of genes identified as modulated by gRNA 9-1 was heat shock chaperones. Specifically, HSP31-34 heat shock proteins are homologs of the human DJ-1/PARK7 gene, in which autosomal recessive mutations have been shown to be associated with early onset of familial PD (57-59). DJ-1 is thought to protect neurons from mitochondrial oxidative stress by acting as a redox-dependent chaperone to inhibit αSyn aggregates (58, 60). As homologs of DJ-1, the roles of HSP31-34 in protecting yeast cells from αSyn toxicity have been previously investigated (61); however, these genes have not been identified in previous genome-wide screens for modifiers of αSyn toxicity. SNO4/HSP34 and HSP32 were identified as two of the genes that were differentially expressed in the screen described herein. As shown in FIGS. 2A-2C, expression of both SNO4/HSP34 and HSP32 significantly rescued αSyn-induced growth defects and membrane-trafficking abnormalities when over-expressed. Interestingly, SNO4/HSP34 was moderately up-regulated by gRNA 9-1, whereas HSP32 was extremely down-regulated. (FIG. 1C and Table 3), which could reflect evolutionary conserved functions of these paralog proteins, despite being under control of different gene regulation programs. Furthermore, overexpression of the other two yeast DJ-1 homologs (HSP31 and HSP33) also significantly suppressed αSyn toxicity (FIG. 2A), even though they were not found to be significantly modulated by gRNA 9-1. This further supports the involvement of this class of paralog heat-shock proteins in suppressing αSyn toxicity. Consistently, HSP31 (which is the least conserved gene with DJ-1 among HSP31-34) was recently shown as a chaperone involved in mitigating various protein misfolding stresses, including αSyn (62).

Among other top αSyn-toxicity suppressors (Table 3 and FIGS. 2A-2C), yeast SAF1 encodes an F-Box protein that selectively targets unprocessed vacuolar/lysosomal proteins for proteasome-dependent degradation (63, 64). The homolog of this protein in mice and humans, ALS2/alsin, functions as a guanine nucleotide exchange factor (GEF) that activates the small GTPase Rab5, an evolutionally conserved protein involved in membrane trafficking in endocytic pathways (65). Mutations in human ALS2 have been shown to cause autosomal recessive motor neuron diseases (66). In addition, it was found that GGA1 and its paralog GGA2 could both ameliorate αSyn toxicity (FIGS. 2A-3C and FIG. 8), neither of which had been previously reported to be associated with suppression of αSyn toxicity. Yeast GGA1 protein has been implicated in binding ubiquitin to facilitate the sorting of cargo proteins from the trans-Golgi network to endosomal compartments (67, 68). Human GGA1 over-expression attenuates amyloidogenic processing of the amyloid precursor proteins (APP) in Alzheimer's disease and a rare inherited lipid-storage disease, Niemann-Pick type C (NPC) (69, 70). Finally, the yeast Hsp40 homolog of human DNAJ/HSP40 family proteins, SIS1, was identified as a novel αSyn suppressor via our crisprTF screening approach. DNAJ family proteins play roles in priming the specificity of HSP70 chaperoning complexes. It has been shown that mammalian DNAJ and HSP70 are up-regulated in response to αSyn overexpression (71). In addition, the DNAJB subfamily has been shown to suppress polyglutamine (polyQ) aggregates (72). These results demonstrate that bi-directional transcriptional perturbations with crisprTF enable the discovery of modulators of disease-relevant phenotypes.

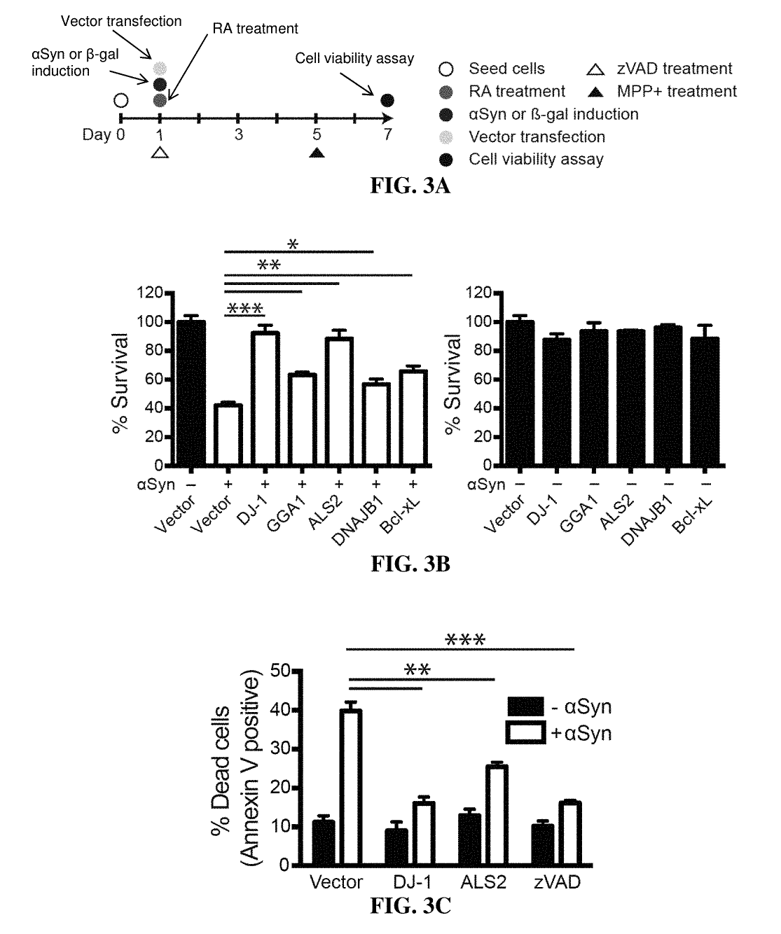

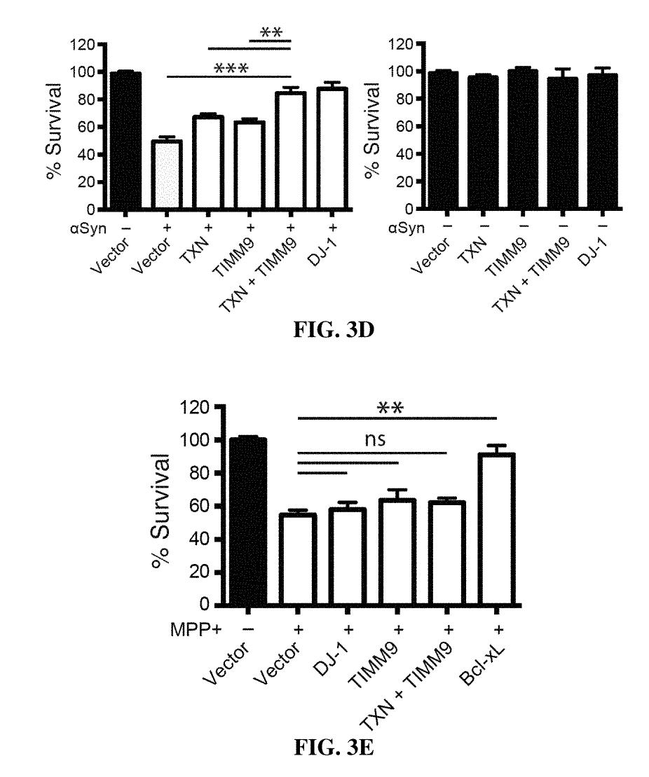

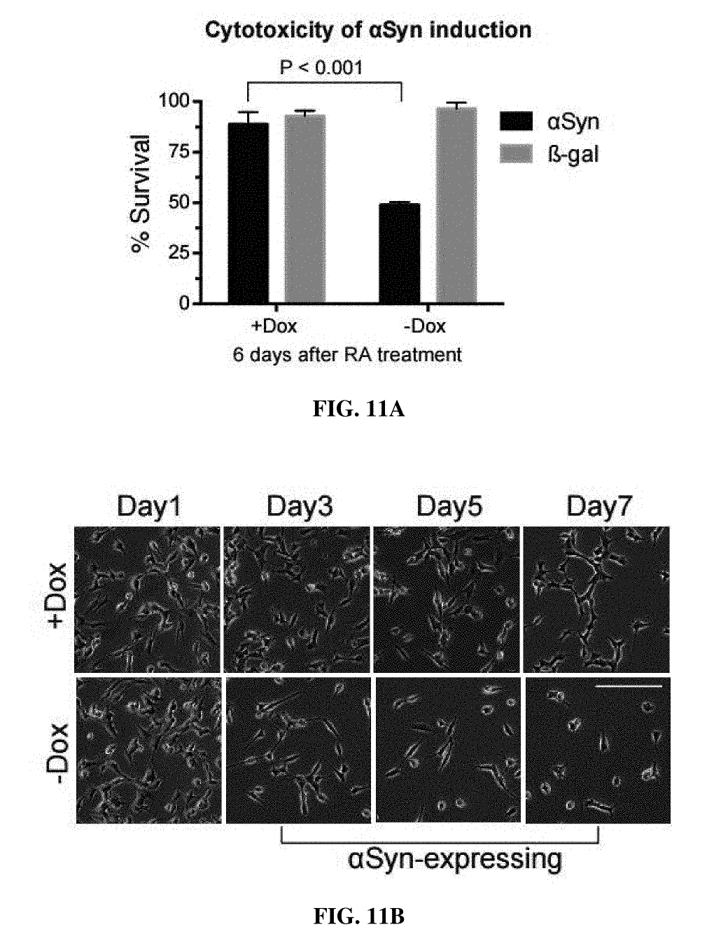

The neuroprotective effects of human homologs of the yeast genes that were identified as having protective effects were investigated. Briefly, DJ-1, ALS2, GGA1, and DNAJB1 were over-expressed in an αSyn-overexpressing human neuroblastoma cell line (SH-SY5Y), an established neural model of PD (73). SH-SY5Y cells were differentiated into cells with dopaminergic neuron-like phenotypes upon retinoic acid (RA) treatment. When ß-galactosidase (ß-gal) was expressed in these cells, no toxicity was observed, however expression of αSyn resulted in gradual neurite retraction and 40-50% viability at 6 days after differentiation (FIGS. 11A and 11B). Expressing DJ-1 or ALS2 alone did not alter cell survival in the absence of αSyn, but strongly suppressed αSyn-inducible cell death (FIG. 3B). αSyn-expressing cells that were transfected with GGA1 or DNAJB1 exhibited approximately 60% viability, which was similar to the effect of expressing the known anti-apoptotic gene, Bcl-xL (positive control). Consistent with these results, overexpression of DJ-1 and ALS2 resulted in a reduction in the population of dead cells, as did treatment with the apoptotic inhibitor zVAD (FIG. 3C).

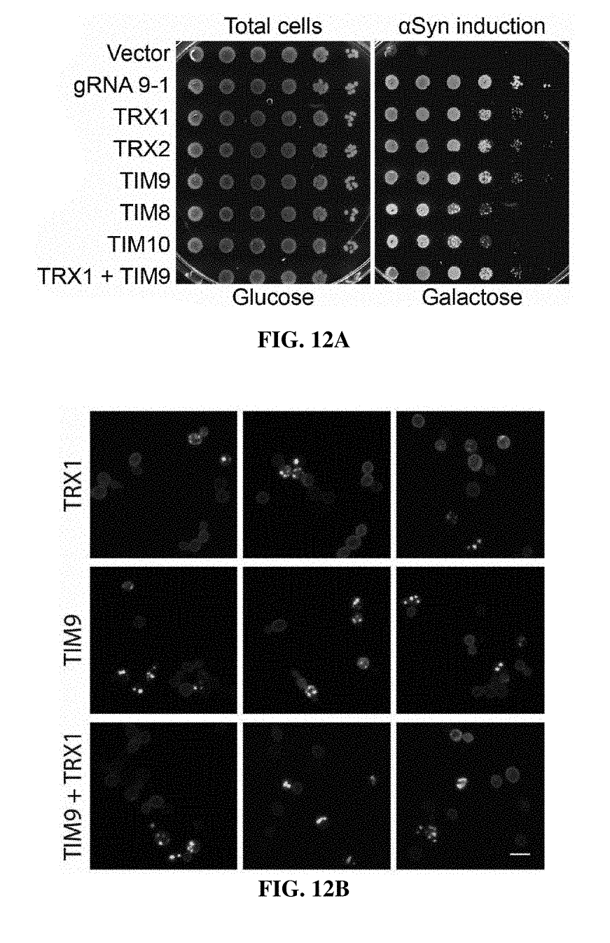

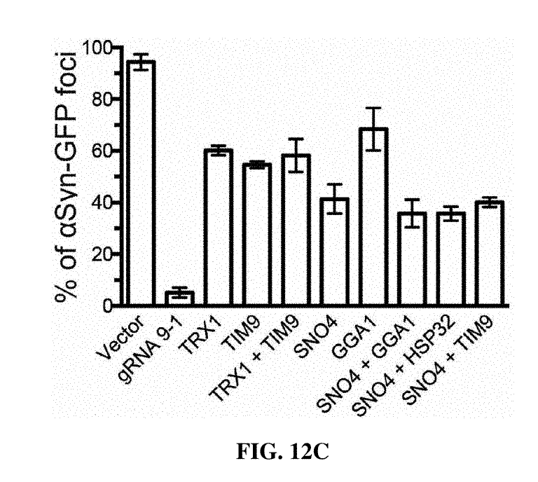

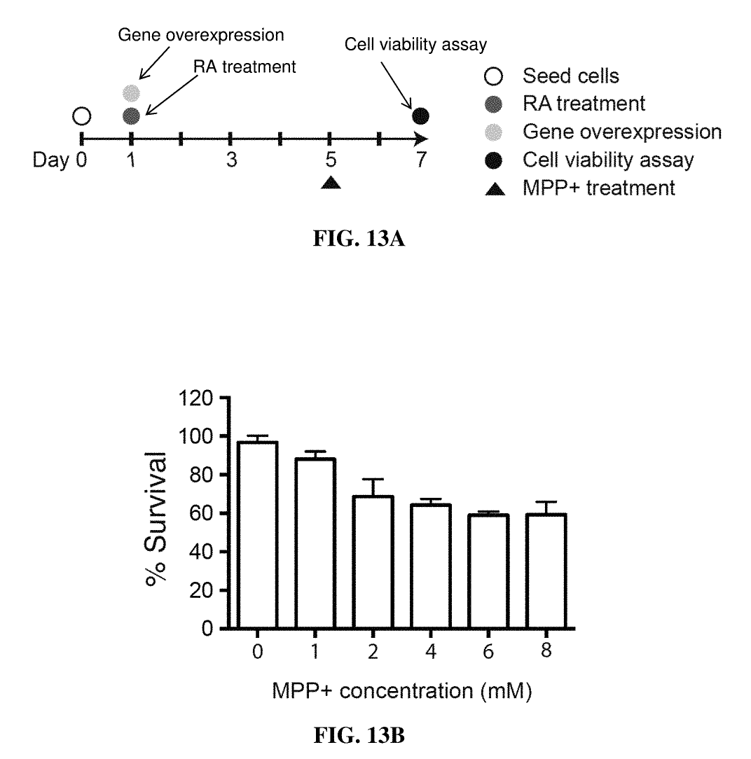

Increased oxidative stresses and defective mitochondrial function are pathological mechanisms involved in sporadic PD (74). The yeast thioredoxin TRX1, a oxidoreductase involved in the maintenance of the cellular redox potential and TIM9, a mitochondrial chaperone involved in the transport of hydrophobic proteins across mitochondrial intermembrane space (75), were both identified as participating in the suppression of αSyn toxicity in yeast cells (FIGS. 2A-2C and FIGS. 12A-12C). Neuronal cells transfected with the human homologs of these genes, TXN or TIMM9, exhibited about ˜60% survival upon αSyn induction as compared with <50% survival observed with the vector control expressing no transgene. Intriguingly, co-expression of TXN and TIMM9 led to enhanced survival in the presence of αSyn induction (˜88% survival) (FIG. 3D). Furthermore, the neuroprotective effects of expressing DJ-1, TXN, and TIMM9 were specific to αSyn-associated toxicity, as these genes did not protect against 1-methyl-4-phenyl pyridinium (MPP+) induced neurodegeneration (76) (FIG. 3E and FIG. 13B).

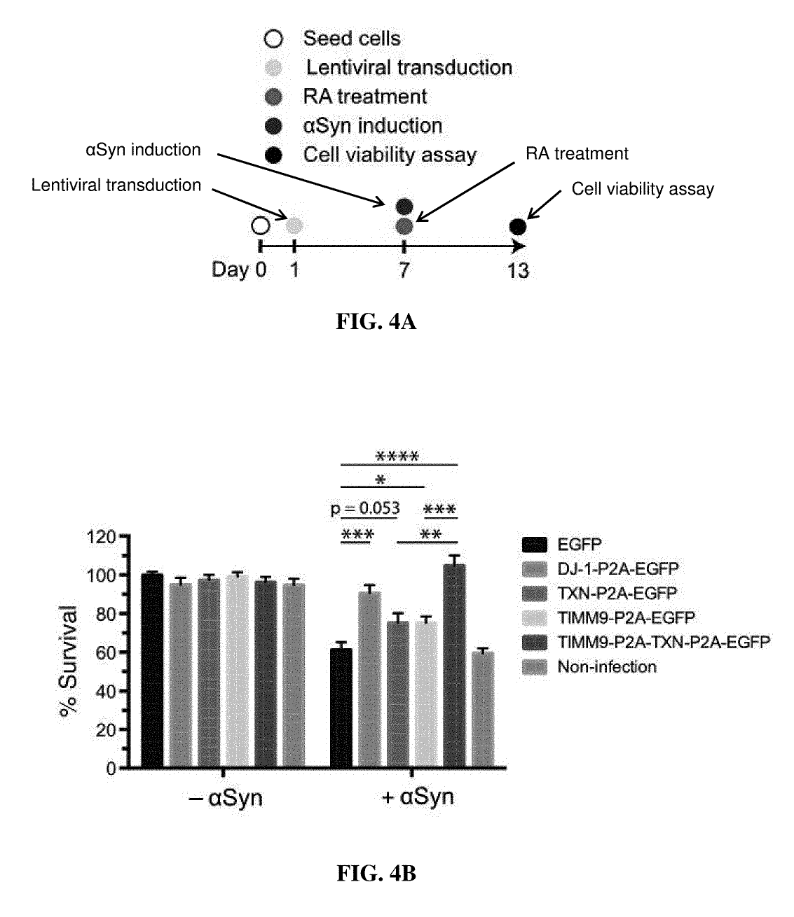

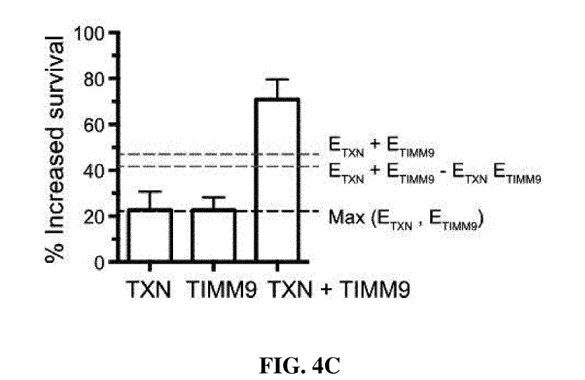

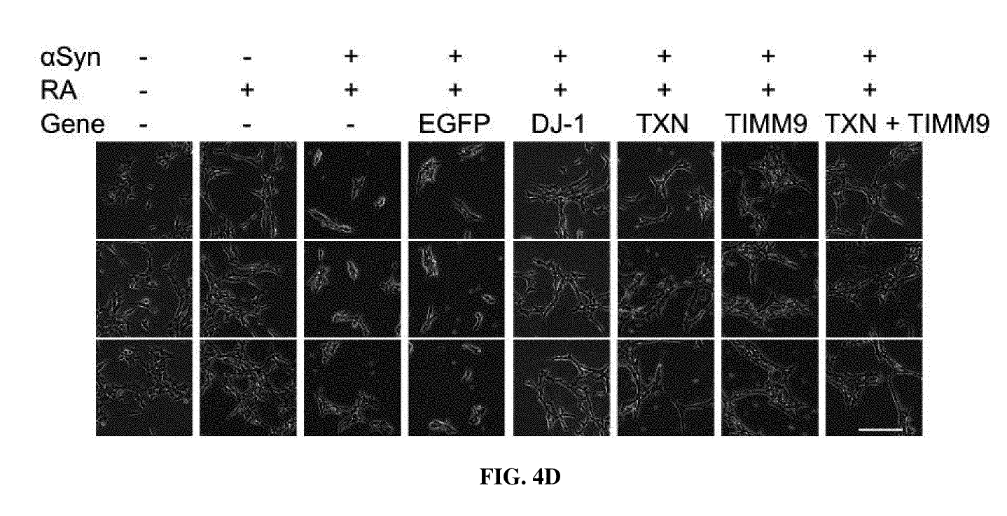

To further investigate these novel genes as potential therapeutic targets for neuroprotection in PD, lentiviral vectors were engineered to express DJ-1, TXN and TIMM9, and to co-express TXN and TIMM9. These vectors were then used to stably infect cells prior to inducing αSyn stress. Consistent with the transient transfection experiments, DJ-1 reliably prevented differentiated SH-SY5Y cells from αSyn-induced cell death and neuronal abnormalities, as did co-expression of TXN and TIMM9 (FIG. 4). These results also suggest that activation of these endogenous genes or enhanced expression and/or activity could present therapeutic targets for neuroprotection in PD.

### Methods

### Yeast Strains and Growth Conditions

Strains used in this study are all derivatives of W303 (MATa ade2-1 trp1-1 can1-100 leu2-3, 112 his3-11, 15 ura3). The ITox2C yeast strain (54) harboring two copies of αSyn (WT)-YFP under control of the Gal-inducible GAL) promoter (hereafter referred to as the parental strain, a generous gift from Dr. Susan Lindquist, Whitehead Institute, USA) was used for the construction of the crisprTF-expressing screening strain. The Dox-inducible (Tet-ON) promoter was constructed by cloning the pTRE promoter and reverse tetracycline-controlled transactivator (rtTA, from Addgene plasmid #31797) upstream of a minimal pCYC1 promoter in the pRS305 backbone. The dCas9-VP64 expression cassette was then cloned into this vector using Gibson assembly. A sense mutation was introduced within the LEU2 ORF by using the QuikChange system (Stratagene) in order to generate a unique PstI site in the vector. The pRS305-pTet-ON-dCas9-VP64 plasmid was linearized by PstI and transformed into ITox2C parental strain to build the screen strain. Leucine-positive integrants were verified by genomic PCRs as well as testing for the presence of αSyn-mediated defects by the survival assay and microscopy after Gal induction.

To build the GAL4* strain, a sequence containing full endogenous GAL4 promoter (−257 to 214) was first PCR amplified by oligos (forward: 5′-CCCAGTATTTTTTTTATTCTACAAACC-3′(SEQ ID NO: 7); reverse: 5′-AAATCAGTAGAAATAGCTGTTCCAGTCTTCTAGCCTTGATTCCACTTCTGTCAGg TGaGCtCggGTtaaCGGAGACCTTTTGGTTTGG-3′ (SEQ ID NO: 8)). This fragment was then assembled (by Gibson assembly) with a kanMX6 expression cassette amplified from pFA6a-kanMX (Addgene plasmid #39296) using oligos (forward: 5′-GGGGCGATTGGTTTGGGTGCGTGAGCGGCAAGAAGTTTCAAAACGTCCGCGTCC TTTGAGACAGCATFCGGAATTCGAGCTCGTTTAAAC-3′ (SEQ ID NO: 9); reversed: 5′-GAAGGTTTGTAGAATAAAAAAAATACTGGGCGGATCCCCGGGTTAATTAA-3′ (SEQ ID NO: 10)). The assembled kanMX-GAL4* cassette was then purified and transformed into yeast cells and transformants were selected in presence of 200 mg/L G418 (Thermo Fisher Scientific). Integrants were confirmed by yeast colony PCR and Sanger sequencing.

Yeast cells were cultured in either YPD (1% yeast extract, 2% Bacto-peptone and 2% glucose) or Synthetic complete medium (Scm) supplemented with 2% glucose, raffinose, or galactose. Doxycycline (Sigma) was added directly to culture media or plates immediately before pouring (final concentration of 1 μg/mL).

Randomized gRNA Library Construction and Screening

To build the randomized gRNA library, random oligonucleotides containing 20 bp random nucleotides flanked by homology arms to the vector were co-transformed into yeast with a linearized 2μ vector flanked by RPR1 promoter and gRNA handle at the ends into the screen yeast strain. Once inside the cells, a gRNA-expressing library was reconstituted by the yeast homologous recombination machinery. The GC content of the randomized portion of the oligo pool was set to 64% to match with the average GC content of yeast promoters. The libraries were screened in the presence of both gal and Dox, and the gRNA content of surviving colonies were characterized by colony PCR followed by Sanger sequencing. Individual gRNAs were verified by cloning each gRNA sequence into the empty gRNA vector and transforming these vectors back into the screen strain to validate gRNA activity in a clean background.

### Yeast Growth and Viability Assays

The yeast screen strain was transformed with gRNAs or individual genes obtained from yeast ORF library. Single transformant colonies were grown overnight in Scm−Uracil (Ura)+raffinose media in the presence of Dox (1 μg/mL) to induce crisprTF expression. Saturated cultures were diluted to OD₆₀₀=0.1 in Scm−Ura+Glucose+Dox and Scm−Ura+Galactose+Dox media and grown at 30° C. in a Synergy H1 Microplate Reader (BioTek). OD₆₀₀ and fluorescence (excitation and emission spectrum at 508 and 534 nm, respectively) were monitored over the course of the experiments. For measuring cell viability by spotting assays, cultures were serially diluted (5-fold dilutions) and spotted on Scm−Ura+Glucose+Dox plates for visualizing total viable cells and on Scm−Ura+Galactose+Dox plates for measuring survival. Plates were incubated at 30° C. for 2 days. An arbitrary score was used to score survival; cells expressing the empty vector (that showed the least survival upon αSyn induction) were scored as 1, and the samples showing the highest survival (those expressing gRNA 9-1) were scored as 5. Other samples were scored by visual inspection and comparing the spotting assay survival results with the two abovementioned reference points.

### Potential Target Site Analysis

Potential target sites for gRNAs 6-3 and 9-1 in the S. cerevisiae genome were identified using CasOT CRISPR off-target search tool (84). All potential target sites with up to two mismatches inside the seed region are presented in Table 2.

### RNA Preparation and Sequencing

The screen S. cerevisiae strain was transformed with either a vector expressing gRNA 9-1 or the empty gRNA vector. Two single-colony transformants from each sample were grown overnight in Scm−Ura+Glucose+Dox. These cultures were diluted into the same fresh media to OD₆₀₀=0.1 and were incubated at 30° C., 300 RPM. Samples were collected in mid-logarithmic phase (OD₆₀₀=0.8) and flash-frozen in liquid nitrogen. Samples were kept in −80° C. until further processing. Total RNA samples were prepared using the MasterPure Yeast RNA Purification kit (Epicentre) following the manufacturer's protocol. mRNA libraries were prepared using the Illumina TruSeq library preparation kit, barcoded, multiplexed and sequenced by Illumina HiSeq. The reads were processed by the MIT BioMicroCenter facility pipeline and mapped to the S. cerevisiae reference genome (sacCer3). RPKM values were calculated using ArrayStar and differentially expressed genes were identified by t-test (p-value≤0.1, FDR correction (85)). Genes that exhibited at least twofold changes in expression in cells containing the gRNA 9-1 compared with the reference (empty gRNA vector) were considered as differentially expressed. Functional classification of the identified genes was performed using the FunSpec webserver (86).

### Expression and Fluorescence Imaging

Yeast protein extracts were prepared for Western blotting by trichloroacetic acid extraction. Blots were probed in phosphate-buffered saline containing 0.1% Tween containing 1% (w/v) dried milk. Overexpression constructs containing a 6×His tag were detected using anti-His monoclonal antibody (1:2000; R93025, Life Technologies) followed by anti-mouse-HRP secondary antibody. αSyn (SNCA) was detected with mouse monoclonal anti-αSyn antibodies (1:1000; Syn-1, BD Biosciences).

The expression level of genes, such as GAL4A, SNCA (αSyn) and ACT1 was performed using RT-PCR with gene-specific primers. Briefly, overnight cultures of the yeast strains were grown in glucose and galactose media for 3 or 6 hours. Total RNA was extracted from these samples, and the gene expression analyzed. Quantitative real-time PCR performed with the gene-specific provided in Table 6.

TABLE 6
Primers for RT-PCR and real-time PCR

| Primer | | Length |
|---|---|---|
| Name | Oligo Sequence | (nt) |
| GAL4_qF1 | 5′- GGTCTTCGAGTCAGGTTCCA -3′ | 20 |
| | (SEQ ID NO: 11) | |
| GAL4_qR1 | 5′- CGGCGTCTTTGTTCCAGAAT -3′ | 20 |
| | (SEQ ID NO: 12) | |
| SNCA_qF1 | 5′- CAAACAGGGTGTGGCAGAAG -3′ | 20 |
| | (SEQ ID NO: 13) | |
| SNCA_qR1 | 5′- CTCCCTCCACTGTCTTCTGG -3′ | 20 |
| | (SEQ ID NO: 14) | |
| ACT1_qF1 | 5′- CGAATTGAGAGTTOCCCCAG -3′ | 20 |
| | (SEQ ID NO: 15) | |
| ACT1_qR1 | 5′- CAAGGACAAAACGGCTTGGA -3′ | 20 |
| | (SEQ ID NO: 16) | |

αSyn-YFP expressing cells were directly visualized under an inverted fluorescence microscope (Zeiss) after 6 days of αSyn induction. The phenotypes were quantified by counting αSyn foci in at least 100 individual cells in multiple randomly chosen fields of view for three independent sets of experiments.

### Neuroblastoma Cell Culture and Gene Expression

Parental and engineered SH-SY5Y cell lines (73) (kindly provided by Dr. Leonidas Stefanis, Biomedical Research Foundation Academy Of Athens, Greece) were grown in Dulbecco's Modified Eagle Medium/Nutrient Mixture F-12 (DMEM/F-12) base medium plus 1% GlutaMAX™ (Gibco) supplemented with 15% heat-inactivated FBS (Fetal Bovine Serum) and 1× antibiotic-antimycotic (Life Technologies) at 37° C. with 5% CO2. Cells were seeded at an initial density of 10⁴ cells/cm² in culture dishes coated with 0.05 mg/mL collagen (Invitrogen). Cells were maintained with 2 μg/mL Dox as previously described (73), in order to repress expression of αSyn and ß-galactosidase (ß-gal), which are driven by the Tet-OFF promoter (73, 87). The expression of αSyn and ß-gal was induced by removing Dox from the media. Cells were differentiated by treating the cells with 10 μM all-trans Retinal (RA; Sigma) for 6 days. For transient expression of human genes, cells were transfected by adding 1 μg plasmid DNA/4 μL FuGENE® HD Transfection Reagent (Promega).

Lentivirus production and transduction were performed as previously described (88). Viral supernatants from 293 fibroblasts were collected at 48-hr after transfection, and filtered through a 0.45 μm polyethersulfone membrane. For transduction with individual vector constructs, 2 ml filtered viral supernatant was used to infect 2×10⁶ cells in the presence of 8 μg/mL polybrene (Sigma) overnight. Cells were washed with fresh culture medium 1 day after infection, and cultured for following 6 days before RA treatment and αSyn induction.

### Neuroblastoma Cell Viability and Death Assays

Viable SH-SY5Y cells were quantified by using the CellTiter-Glo Luminescent Cell Viability Assay (Promega). Images were captured using the EVOS™ FL Cell Imaging System directly from culture plates under 10× magnification. Cell death was measured by the FITC Annexin V Apoptosis Detection Kit I (BD Pharmingen™) followed by flow cytometry analysis. At least 10,000 cells were recorded per sample in each data set. In the cell death assay (FIG. 3C), caspase inhibitor zVAD (Z-VAD-FMK; BD Biosciences) was added into the media upon αSyn induction (100 μM final concentration). For the cell survival assay (FIG. 3E), MPP+ iodide (1-Methyl-4-phenylpyridinium iodide; Sigma) was added into media of transfected cells 48 hours before processing for cell viability assay.

### Synergy Quantification

The increased suppression of αSyn toxicity by overexpression of TXN, TIMM9, and TXN+TIMM9 was normalized to the vector control (FIG. 3D) or the EGFP control (FIG. 4B). Co-expression of TXN+TIMM9 to be interacting synergistically if the observed combination effect was greater than the expected effect given by Highest Single Agent (81), Linear Interaction Effect (82), and Bliss Independence (83) models. Synergy was calculated based on data presented in FIG. 4B and tested by three models respectively, as illustrated in FIG. 4C.

### REFERENCES

1. A. L. Brass et al., Identification of host proteins required for HIV infection through a functional genomic screen. Science 319, 921-926 (2008).
2. J. E. Carette et al., Haploid genetic screens in human cells identify host factors used by pathogens. Science 326, 1231-1235 (2009).
3. L. M. Duncan et al., Fluorescence-based phenotypic selection allows forward genetic screens in haploid human cells. PLoS One 7, e39651 (2012).
4. J. Moffat et al., A lentiviral RNAi library for human and mouse genes applied to an arrayed viral high-content screen. Cell 124, 1283-1298 (2006).
5. R. D. Paulsen et al., A genome-wide siRNA screen reveals diverse cellular processes and pathways that mediate genome stability. Mol Cell 35, 228-239 (2009).
6. M. Pritsker, N. R. Ford, H. T. Jenq. I. R. Lemischka, Genomewide gain-of-function genetic screen identifies functionally active genes in mouse embryonic stem cells. Proc Natl Acad Sci USA 103, 6946-6951 (2006).
7. D. E. Root, N. Hacohen, W. C. Hahn, E. S. Lander, D. M. Sabatini, Genome-scale loss-of-function screening with a lentiviral RNAi library. Nat Methods 3, 715-719 (2006).
8. A. W. Whitehurst et al., Synthetic lethal screen identification of chemosensitizer loci in cancer cells. Nature 446, 815-819 (2007).
9. K. Demir, M. Boutros, Cell perturbation screens for target identification by RNAi. Methods Mol Biol 910, 1-13 (2012).
10. C. N. Santos, G. Stephanopoulos, Combinatorial engineering of microbes for optimizing cellular phenotype. Curr Opin Chem Biol 12, 168-176 (2008).
11. A. Beltran, Y. Liu, S. Parikh, B. Temple. P. Blancafort, Interrogating genomes with combinatorial artificial transcription factor libraries: asking zinc finger questions. Assay Drug Dev Technol 4, 317-331 (2006).
12. P. Blancafort et al., Genetic reprogramming of tumor cells by zinc finger transcription factors. Proc Natl Acad Sci USA 102, 11716-11721 (2005).
13. K. S. Park et al., Phenotypic alteration of eukaryotic cells using randomized libraries of artificial transcription factors. Nat Biotechnol 21, 1208-1214 (2003).
14. A. Chavez et al., Highly efficient Cas9-mediated transcriptional programming. Nat Methods 12, 326-328 (2015).
15. F. Farzadfard, S. D. Perli, T. K. Lu, Tunable and Multifunctional Eukaryotic Transcription Factors Based on CRISPR/Cas. ACS Synth Biol, (2013).
16. S. Konermann et al., Genome-scale transcriptional activation by an engineered CRISPR-Cas9 complex. Nature 517, 583-588 (2015).
17. P. Mali et al., CAS9 transcriptional activators for target specificity screening and paired nickases for cooperative genome engineering. Nat Biotechnol 31, 833-838 (2013).
18. H. Nishimasu et al., Crystal structure of Cas9 in complex with guide RNA and target DNA. Cell 156, 935-949 (2014).
19. L. S. Qi et al., Repurposing CRISPR as an RNA-guided platform for sequence-specific control of gene expression. Cell 152, 1173-1183 (2013).
20. M. E. Tanenbaum, L. A. Gilbert, L. S. Qi, J. S. Weissman, R. D. Vale, A protein-tagging system for signal amplification in gene expression and fluorescence imaging. Cell 159, 635-646 (2014).
21. J. G. Zalatan et al., Engineering complex synthetic transcriptional programs with CRISPR RNA scaffolds. Cell 160, 339-350 (2015).
22. Y. Zhao et al., Sequence-specific inhibition of microRNA via CRISPR/CRISPRi system. Scientific reports 4, 3943 (2014).
23. L. A. Gilbert et al., Genome-Scale CRISPR-Mediated Control of Gene Repression and Activation. Cell 159, 647-661 (2014).
24. M. A. Horlbeck et al., Compact and highly active next-generation libraries for CRISPR-mediated gene repression and activation. eLife 5, (2016).
25. A. S. Wong, G. C. Choi, A. A. Cheng, O. Purcell, T. K. Lu, Massively parallel high-order combinatorial genetics in human cells. Nat Biotechnol 33, 952-961 (2015).
26. A. S. Wong et al., Multiplexed barcoded CRISPR-Cas9 screening enabled by CombiGEM. Proc Nat Acad Sci USA 113, 2544-2549 (2016).
27. O. Parnas et al., A Genome-wide CRISPR Screen in Primary Immune Cells to Dissect Regulatory Networks. Cell 162, 675-686 (2015).
28. O. Shalem et al., Genome-scale CRISPR-Cas9 knockout screening in human cells. Science 343, 84-87 (2014).
29. R. Cencic et al., Protospacer adjacent motif (PAM)-distal sequences engage CRISPR Cas9 DNA target cleavage. PLoS One 9, e109213 (2014).
30. R. L. Frock et al., Genome-wide detection of DNA double-stranded breaks induced by engineered nucleases. Nat Biotechnol 33, 179-186 (2015).
31. H. O'Geen, I. M. Henry, M. S. Bhakta, J. F. Meckler, D. J. Segal. A genome-wide analysis of Cas9 binding specificity using ChIP-seq and targeted sequence capture. Nucleic acids research 43, 3389-3404 (2015).
32. X. Wu et al., Genome-wide binding of the CRISPR endonuclease Cas9 in mammalian cells. Nat Biotechnol 32, 670-676 (2014).
33. T. Wang, J. J. Wei, D. M. Sabatini, E. S. Lander, Genetic screens in human cells using the CRISPR-Cas9 system. Science 343, 80-84 (2014).
34. M. Goedert, M. G. Spillantini, K. Del Tredici, H. Braak, 100 years of Lewy pathology. Nature reviews. Neurology 9, 13-24 (2013).
35. M. G. Spillantini et al., Alpha-synuclein in Lewy bodies. Nature 388, 839-840 (1997).
36. F. L. Campos et al., Rodent models of Parkinson's disease: beyond the motor symptomatology. Front Behav Neurosci 7, 175 (2013).
37. M. F. Chesselet et al., A progressive mouse model of Parkinson's disease: the Thy1-aSyn (“Line 61”) mice. Neurotherapeutics 9, 297-314 (2012).
38. S. E. Davies et al., Enhanced ubiquitin-dependent degradation by Nedd4 protects against alpha-synuclein accumulation and toxicity in animal models of Parkinson's disease. Neurobiol Dis 64, 79-87 (2014).
39. V. Franssens et al., The benefits of humanized yeast models to study Parkinson's disease. Oxid Med Cell Longev 2013, 760629 (2013).
40. M. Hollerhage et al., Trifluoperazine rescues human dopaminergic cells from wild-type alpha-synuclein-induced toxicity. Neurobiol Aging, (2014).
41. L. Li et al., Human A53T alpha-synuclein causes reversible deficits in mitochondrial function and dynamics in primary mouse cortical neurons. PLoS One 8, e85815 (2013).
42. A. Ray, B. A. Martinez, L. A. Berkowitz, G. A. Caldwell, K. A. Caldwell, Mitochondrial dysfunction, oxidative stress, and neurodegeneration elicited by a bacterial metabolite in a C. elegans Parkinson's model. Cell Death Dis 5, e984 (2014).
43. K. J. Spinelli et al., Presynaptic alpha-synuclein aggregation in a mouse model of Parkinson's disease. J Neurosci 34, 2037-2050 (2014).
44. D. F. Tardiff et al., Yeast reveal a “druggable” Rsp5/Nedd4 network that ameliorates alpha-synuclein toxicity in neurons. Science 342, 979-983 (2013).
45. C. Y. Chung et al., Identification and rescue of alpha-synuclein toxicity in Parkinson patient-derived neurons. Science 342, 983-987 (2013).
46. G. Ciaccioli, A. Martins, C. Rodrigues, H. Vieira, P. Calado, A powerful yeast model to investigate the synergistic interaction of alpha-synuclein and tau in neurodegeneration. PLoS One 8, e55848 (2013).
47. S. Buttner et al., The Ca2+/Mn2+ ion-pump PMR1 links elevation of cytosolic Ca(2+) levels to alpha-synuclein toxicity in Parkinson's disease models. Cell Death Differ 20, 465-477 (2013).
48. D. Petroi et al., Aggregate clearance of alpha-synuclein in Saccharomyces cerevisiae depends more on autophagosome and vacuole function than on the proteasome. J Biol Chem 287, 27567-27579 (2012).
49. M. Usenovic et al., Identification of novel ATP13A2 interactors and their role in alpha-synuclein misfolding and toxicity. Hum Mol Genet 21, 3785-3794 (2012).
50. A. Chesi, A. Kilaru, X. Fang, A. A. Cooper, A. D. Gitler, The role of the Parkinson's disease gene PARK9 in essential cellular pathways and the manganese homeostasis network in yeast. PLoS One 7, e34178 (2012).
51. E. Swinnen et al., Aggresome formation and segregation of inclusions influence toxicity of alpha-synuclein and synphilin-1 in yeast. Biochem Soc Trans 39, 1476-1481 (2011).
52. J. H. Soper, V. Kehm, C. G. Burd, V. A. Bankaitis, V. M. Lee. Aggregation of alpha-synuclein in S. cerevisiae is associated with defects in endosomal trafficking and phospholipid biosynthesis. J Mol Neurosci 43, 391-405 (2011).
53. V. Khurana, S. Lindquist, Modelling neurodegeneration in Saccharomyces cerevisiae: why cook with baker's yeast? Nature reviews. Neuroscience 11, 436-449 (2010).
54. A. A. Cooper et al., Alpha-synuclein blocks ER-Golgi traffic and Rab1 rescues neuron loss in Parkinson's models. Science 313, 324-328 (2006).
55. A. D. Gitler et al., Alpha-synuclein is part of a diverse and highly conserved interaction network that includes PARK9 and manganese toxicity. Nature genetics 41, 308-315 (2009).
56. T. F. Outeiro, S. Lindquist, Yeast cells provide insight into alpha-synuclein biology and pathobiology. Science 302, 1772-1775 (2003).
57. H. Ariga et al., Neuroprotective function of DJ-1 in Parkinson's disease. Oxid Med Cell Longev 2013, 683920 (2013).
58. V. Bonifati et al., Mutations in the DJ-1 gene associated with autosomal recessive early-onset parkinsonism. Science 299, 256-259 (2003).
59. X. Xu, F. Martin, J. S. Friedman, The familial Parkinson's disease gene DJ-1 (PARK7) is expressed in red cells and plays a role in protection against oxidative damage. Blood cells, molecules & diseases 45, 227-232 (2010).
60. R. M. Canet-Aviles et al., The Parkinson's disease protein DJ-1 is neuroprotective due to cysteine-sulfinic acid-driven mitochondrial localization. Proc Natl Acad Sci USA 101, 9103-9108 (2004).
61. L. Zondler et al., DJ-1 interactions with alpha-synuclein attenuate aggregation and cellular toxicity in models of Parkinson's disease. Cell Death Dis 5, e1350 (2014).
62. C. J. Tsai et al., Hsp31 Is a Stress Response Chaperone That Intervenes in the Protein Misfolding Process. J Biol Chem 290, 24816-24834 (2015).
63. S. Escusa et al., Skp1-Cullin-F-box-dependent degradation of Aah1p requires its interaction with the F-box protein Saf1p. J Biol Chem 282, 20097-20103 (2007).
64. K. G. Mark, M. Simonetta, A. Maiolica, C. A. Seller, D. P. Toczyski, Ubiquitin ligase trapping identifies an SCF(Saf1) pathway targeting unprocessed vacuolar/lysosomal proteins. Mol Cell 53, 148-161 (2014).
65. S. Hadano, R. Kunita, A. Otomo, K. Suzuki-Utsunomiya, J. E. Ikeda, Molecular and cellular function of ALS2/alsin: implication of membrane dynamics in neuronal development and degeneration. Neurochemistry international 51, 74-84 (2007).
66. J. Chandran. J. Ding. H. Cai, Alsin and the molecular pathways of amyotrophic lateral sclerosis. Molecular neurobiology 36, 224-231 (2007).
67. H. Takatsu, K. Yoshino, K. Toda, K. Nakayama, GGA proteins associate with Golgi membranes through interaction between their GGAH domains and ADP-ribosylation factors. The Biochemical journal 365, 369-378 (2002).
68. O. Zhdankina, N. L. Strand, J. M. Redmond, A. L. Boman, Yeast GGA proteins interact with GTP-bound Arf and facilitate transport through the Golgi. Yeast (Chichester, England) 18, 1-18 (2001).
69. M. Kosicek. P. Wunderlich, J. Walter, S. Hecimovic. GGA1 overexpression attenuates amyloidogenic processing of the amyloid precursor protein in Niemann-Pick type C cells. Biochemical and biophysical research communications 450, 160-165 (2014).
70. B. von Einem et al., The Golgi-Localized gamma-Ear-Containing ARF-Binding (GGA) Proteins Alter Amyloid-beta Precursor Protein (APP) Processing through Interaction of Their GAE Domain with the Beta-Site APP Cleaving Enzyme 1 (BACE1). PLoS One 10, e0129047 (2015).
71. M. J. Vos, J. Hageman, S. Carra, H. H. Kampinga, Structural and functional diversities between members of the human HSPB, HSPH, HSPA, and DNAJ chaperone families. Biochemistry 47, 7001-7011 (2008).
72. J. Gillis et al., The DNAJB6 and DNAJB8 protein chaperones prevent intracellular aggregation of polyglutamine peptides. J Biol Chem 288, 17225-17237 (2013).
73. K. Vekrellis, M. Xilouri, E. Emmanouilidou, L. Stefanis, Inducible over-expression of wild type alpha-synuclein in human neuronal cells leads to caspase-dependent non-apoptotic death. Journal of neurochemistry 109, 1348-1362 (2009).
74. C. Henchcliffe, M. F. Beal, Mitochondrial biology and oxidative stress in Parkinson disease pathogenesis. Nature clinical practice. Neurology 4, 600-609 (2008).
75. W. Neupert, J. M. Herrmann, Translocation of proteins into mitochondria. Annual review of biochemistry 76, 723-749 (2007).
76. G. P. Dietz et al., Membrane-permeable Bcl-xL prevents MPTP-induced dopaminergic neuronal loss in the substantia nigra. Journal of neurochemistry 104, 757-765 (2008).
77. R. Durigon, Q. Wang, E. Ceh Pavia. C. M. Grant, H. Lu. Cytosolic thioredoxin system facilitates the import of mitochondrial small Tim proteins. EMBO reports 13, 916-922 (2012).
78. V. Dias, E. Junn, M. M. Mouradian, The role of oxidative stress in Parkinson's disease. Journal of Parkinson's disease 3, 461-491 (2013).
79. H. Masutani, J. Bai, Y. C. Kim, J. Yodoi, Thioredoxin as a neurotrophic cofactor and an important regulator of neuroprotection. Molecular neurobiology 29, 229-242 (2004).
80. H. Alper, J. Moxley, E. Nevoigt, G. R. Fink, G. Stephanopoulos, Engineering yeast transcription machinery for improved ethanol tolerance and production. Science 314, 1565-1568 (2006).
81. A. A. Borisy et al., Systematic discovery of multicomponent therapeutics. Proc Natl Acad Sci USA 100, 7977-7982 (2003).
82. B. K. Slinker, The statistics of synergism. Journal of molecular and cellular cardiology 30, 723-731 (1998).
83. W. R. Greco, G. Bravo, J. C. Parsons. The search for synergy: a critical review from a response surface perspective. Pharmacological reviews 47, 331-385 (1995).
84. A. Xiao et al., CasOT: a genome-wide Cas9/gRNA off-target searching tool. Bioinformatics, (2014).
85. Y. Benjamini, and Yosef Hochberg, Controlling the false discovery rate: a practical and powerful approach to multiple testing. Journal of the royal statistical society. Series B (Methodological), 289-300 (1995).
86. M. D. Robinson, J. Grigull, N. Mohammad, T. R. Hughes, FunSpec: a web-based cluster interpreter for yeast. BMC bioinformatics 3, 35 (2002).
87. C. Gouarne et al., Protective role of olesoxime against wild-type alpha-synuclein-induced toxicity in human neuronally differentiated SHSY-5Y cells. British journal of pharmacology 172, 235-245 (2015).
88. C. Lois, E. J. Hong, S. Pease, E. J. Brown, D. Baltimore, Germline transmission and tissue-specific expression of transgenes delivered by lentiviral vectors. Science 295, 868-872 (2002).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more of such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or,” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying.” “having,” “containing,” “involving,” “holding.” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating a neurodegenerative disorder associated with α-synuclein dysfunction, the method comprising
administering to a subject having a disorder associated with α-synuclein dysfunction a therapeutically effective amount of an agent that enhances expression and/or activity of a human homolog of one or more genes set forth in Table 1, optionally wherein if the agent enhances expression of one gene set forth in Table 1, the gene is not heat shock protein (HSP)30, HSP31, HSP32, HSP33, HSP34, UBC8, or YGR130C, or HSP30, HSP31, UBC8, YGR130C or YPL123C (RNY1).

2. The method of claim 1, wherein the gene is selected from the group consisting of YBL086C, YBR056W, SAF1, DAD1, ARX1, ARP10, PET117, STF2, SPL2, YJL144W, TRX1, SRN2, SHH4, ECM19, SNO4, SIS1, DBP2, VHS3, HSP32, GGA1, TIM9, HSP42, YER121W, YGL258W-A, CPD1, YLR149C, NCE103, YOL114C, OXR1, URA7, YDL199C, YKL100C, YMR244W, ATO2, PHM7, PNS1, and YPL247C.

3. The method of claim 1, wherein the human homolog is HSPB1, HSPB3, HSPB6, HSPB7, HSPB8, HSPB9, CRYAA, CRYAB, DNAJB1-B9, GGA1, GGA2, GGA3, TOM1, TOM1L1, TOM1L2, WDFY1, WDFY2, ALS2, RCC1, TXN, TXNDC2, TXNDC8, TIMM9, OXR1, NCOA7, TLDC2, PA2G4, XPNPEP1, XPNPEP2, SDHD, DDX17, DDX41, DDX43, DDX5, DDX53, DDX59, PPCDC, ICT1, CTPS1, CTPS2, HM13, SPPL2A, SPPL2C, SPPL3, TMEM63 (A-C), SLC44 (A1-A5), DCAF7, SERBP1, or HABP4.

4. The method of claim 3, wherein at least two agents that enhance expression and/or activity of TIMM9 and TXN are administered.

5. The method of claim 1, wherein the agent is a small molecule, protein, or a nucleic acid.

6. The method of claim 5, wherein the agent is a gRNA, siRNA, miRNA, shRNA, or a nucleic acid encoding a gene, optionally wherein the agent is encoded on a vector.

7. The method of claim 6, wherein the agent is a nucleic acid encoding a gene, which is a human homolog of one or more of the genes set forth in Table 1.

8. The method of claim 1, wherein the agent is a gRNA and comprises a nucleotide sequence provided by SEQ ID NO: 1 (gRNA 9-1) or SEQ ID NO: 2 (gRNA 6-3).

9. (canceled)

10. The method of claim 1, wherein the agent is administered with a pharmaceutically acceptable excipient.

11. The method of claim 1, wherein the agent is administered in one dose.

12. The method of claim 1, wherein the agent is administered in multiple doses.

13. The method of claim 1, wherein the agent is administered orally, intravenously, intraperitoneally, topically, subcutaneously, intracranially, intrathecally, or by inhalation.

14. The method of claim 1, wherein the disorder associated with α-synuclein dysfunction is Parkinson's disease, Lewy body variant of Alzheimer's disease, diffuse Lewy body disease, dementia with Lewy bodies, multiple system atrophy, or neurodegeneration with brain iron accumulation type I.

15. A nucleic acid comprising the nucleotide sequence provided by SEQ ID NO: 1 (gRNA 9-1) or SEQ ID NO: 2 (gRNA 6-3).

16. A vector comprising the nucleic acid of claim 15.

17. A method for identifying a genetic network involved in regulating a cellular response, comprising
(i) expressing in a population of cells a plurality of randomized guide RNAs and a CRISPR protein;
(ii) culturing the population of cells under conditions that induce the cellular response;
(iii) isolating a subpopulation of cells having an altered readout of the cellular response from the population of cells; and
(iv) identifying a randomized guide RNA present in the cells isolated in (iii) as a guide RNA that regulates a transcriptional network involved in the cellular response.

18. The method of claim 17, wherein the cellular response is α-synuclein toxicity.

19. The method of claim 18, wherein the altered readout of the cellular response is reduced α-synuclein toxicity.

20. The method of claim 17, wherein the randomized guide RNA comprises a plurality of nucleotides, wherein the content of guanine and cytosine nucleotides in the randomized guide RNA is between 50% and 70%.

21. A method for identifying a transcriptional network involved in suppression of α-synuclein toxicity, comprising
(i) expressing in a population of cells a plurality of randomized guide RNAs and a CRISPR-Cas transcription factor;
(ii) culturing the population of cells under conditions of α-synuclein toxicity;
(iii) isolating a subpopulation of cells having suppressed α-synuclein toxicity from the population of cells; and
(iv) identifying a randomized guide RNAs present in the cells isolated in (iii) as a guide RNA that regulates a transcriptional network involved in suppression of α-synuclein toxicity.


SEQUENCE LISTING (171 sequences)

SEQ ID NO: 1 - DNA, 20 bp, Artificial Sequence
  Synthetic polynucleotide
gtataattttccctagtcag

SEQ ID NO: 2 - DNA, 20 bp, Artificial Sequence
  Synthetic polynucleotide
gggtttttcttctatacagg

SEQ ID NO: 3 - DNA, 23 bp, Artificial Sequence
  Synthetic polynucleotide
ccgctgactagggcacatctgac

SEQ ID NO: 4 - protein, 7 aa, Artificial Sequence
  Synthetic polypeptide
PLTRAHL

SEQ ID NO: 5 - DNA, 23 bp, Artificial Sequence
  Synthetic polynucleotide
ggcgactgatcccgtgtagactg

SEQ ID NO: 6 - DNA, 23 bp, Artificial Sequence
  Synthetic polynucleotide
ggcgactgatcccttttaatatg

SEQ ID NO: 7 - DNA, 27 bp, Artificial Sequence
  Synthetic polynucleotide
cccagtattttttttattctacaaacc

SEQ ID NO: 8 - DNA, 90 bp, Artificial Sequence
  Synthetic polynucleotide
aaatcagtagaaatagctgttccagtctttctagccttgattccacttctgtcaggtgagctcgggttaacggagaccttttggttttgg

SEQ ID NO: 9 - DNA, 90 bp, Artificial Sequence
  Synthetic polynucleotide
ggggcgattggtttgggtgcgtgagcggcaagaagtttcaaaacgtccgcgtcctttgagacagcattcggaattcgagctcgtttaaac

SEQ ID NO: 10 - DNA, 50 bp, Artificial Sequence
  Synthetic polynucleotide
gaaggtttgtagaataaaaaaaatactgggcggatccccgggttaattaa

SEQ ID NO: 11 - DNA, 20 bp, Artificial Sequence
  Synthetic polynucleotide
ggtcttcgagtcaggttcca

SEQ ID NO: 12 - DNA, 20 bp, Artificial Sequence
  Synthetic polynucleotide
cggcgtctttgttccagaat

SEQ ID NO: 13 - DNA, 20 bp, Artificial Sequence
  Synthetic polynucleotide
caaacagggtgtggcagaag

SEQ ID NO: 14 - DNA, 20 bp, Artificial Sequence
  Synthetic polynucleotide
ctccctccactgtcttctgg

SEQ ID NO: 15 - DNA, 20 bp, Artificial Sequence
  Synthetic polynucleotide
cgaattgagagttgccccag

SEQ ID NO: 16 - DNA, 20 bp, Artificial Sequence
  Synthetic polynucleotide
caaggacaaaacggcttgga

SEQ ID NO: 17 - DNA, 24 bp, Artificial Sequence
  Synthetic polynucleotide
ggtaatgacttcttgacaggtggc

SEQ ID NO: 18 - DNA, 24 bp, Artificial Sequence
  Synthetic polynucleotide
ttcaacatcttctgtacgggaaga

SEQ ID NO: 19 - DNA, 24 bp, Artificial Sequence
  Synthetic polynucleotide
atgaataccttcaatactggtggt

SEQ ID NO: 20 - DNA, 24 bp, Artificial Sequence
  Synthetic polynucleotide
aggggaatcttgtatacagaaagt

SEQ ID NO: 21 - DNA, 24 bp, Artificial Sequence
  Synthetic polynucleotide
gattattgcttctatattggatgg

SEQ ID NO: 22 - DNA, 24 bp, Artificial Sequence
  Synthetic polynucleotide
tttaaagacttctatagatgaaga

SEQ ID NO: 23 - DNA, 24 bp, Artificial Sequence
  Synthetic polynucleotide
ttcttcttcttctttactgggagt

SEQ ID NO: 24 - DNA, 24 bp, Artificial Sequence
  Synthetic polynucleotide
gcttttcgcttttatacagcagga

SEQ ID NO: 25 - DNA, 24 bp, Artificial Sequence
  Synthetic polynucleotide
ttcgggtacttctaaacagacgga

SEQ ID NO: 26 - DNA, 24 bp, Artificial Sequence
  Synthetic polynucleotide
tatcggaaattctttacaggtagg

SEQ ID NO: 27 - DNA, 24 bp, Artificial Sequence
  Synthetic polynucleotide
tgtagcaccttctatagaggaagt

SEQ ID NO: 28 - DNA, 24 bp, Artificial Sequence
  Synthetic polynucleotide
tgcaatttcttctaaacagtgcgg

SEQ ID NO: 29 - DNA, 24 bp, Artificial Sequence
  Synthetic polynucleotide
gattggagcatctatatagggagc

SEQ ID NO: 30 - DNA, 24 bp, Artificial Sequence
  Synthetic polynucleotide
gatttccacttctgtacagatgga

SEQ ID NO: 31 - DNA, 24 bp, Artificial Sequence
  Synthetic polynucleotide
tcccttagattctgtacaggaaga

SEQ ID NO: 32 - DNA, 24 bp, Artificial Sequence
  Synthetic polynucleotide
tttctcagcttatataaaggatgg

SEQ ID NO: 33 - DNA, 24 bp, Artificial Sequence
  Synthetic polynucleotide
cggaggaacttcaatagaggtaga

SEQ ID NO: 34 - DNA, 24 bp, Artificial Sequence
  Synthetic polynucleotide
gtttgtctcttatatacagccgga

SEQ ID NO: 35 - DNA, 24 bp, Artificial Sequence
  Synthetic polynucleotide
atatccggcttctttgcagggagt

SEQ ID NO: 36 - DNA, 24 bp, Artificial Sequence
  Synthetic polynucleotide
gtttaactcttcaatagaggtcgg

SEQ ID NO: 37 - DNA, 24 bp, Artificial Sequence
  Synthetic polynucleotide
cgtgggttcttctatagagggaga

SEQ ID NO: 38 - DNA, 24 bp, Artificial Sequence
  Synthetic polynucleotide
ctcatatatttgtatacaggaaga

SEQ ID NO: 39 - DNA, 24 bp, Artificial Sequence
  Synthetic polynucleotide
tgaaatcacttctttacaagagga

SEQ ID NO: 40 - DNA, 24 bp, Artificial Sequence
  Synthetic polynucleotide
aacgtaatcttgtataaaggtgga

SEQ ID NO: 41 - DNA, 24 bp, Artificial Sequence
  Synthetic polynucleotide
tttgaaaatttgtatacaggagga

SEQ ID NO: 42 - DNA, 24 bp, Artificial Sequence
  Synthetic polynucleotide
tttagaagcttctattcaagaaga

SEQ ID NO: 43 - DNA, 24 bp, Artificial Sequence
  Synthetic polynucleotide
taaatagacatctatacacgtagt

SEQ ID NO: 44 - DNA, 24 bp, Artificial Sequence
  Synthetic polynucleotide
tatgcttccctccatacaggcagg

SEQ ID NO: 45 - DNA, 24 bp, Artificial Sequence
  Synthetic polynucleotide
ccttcaatcttctatagagccggt

SEQ ID NO: 46 - DNA, 24 bp, Artificial Sequence
  Synthetic polynucleotide
tgtttttaattatatacaggttgg

SEQ ID NO: 47 - DNA, 24 bp, Artificial Sequence
  Synthetic polynucleotide
acgagtcacttctataagggtagg

SEQ ID NO: 48 - DNA, 24 bp, Artificial Sequence
  Synthetic polynucleotide
aaccttgtcttcaatccaggcggc

SEQ ID NO: 49 - DNA, 24 bp, Artificial Sequence
  Synthetic polynucleotide
taaggaaacttctaaacagttcgg

SEQ ID NO: 50 - DNA, 24 bp, Artificial Sequence
  Synthetic polynucleotide
tttctgaccttcaatacatggggg

SEQ ID NO: 51 - DNA, 24 bp, Artificial Sequence
  Synthetic polynucleotide
cctgactttttatatacaggtggc

SEQ ID NO: 52 - DNA, 24 bp, Artificial Sequence
  Synthetic polynucleotide
tcgaggctcttctttaccgggagt

SEQ ID NO: 53 - DNA, 24 bp, Artificial Sequence
  Synthetic polynucleotide
ccttaaaccttctataaatgcaga

SEQ ID NO: 54 - DNA, 24 bp, Artificial Sequence
  Synthetic polynucleotide
tttcaacgcttctaaatagggaga

SEQ ID NO: 55 - DNA, 24 bp, Artificial Sequence
  Synthetic polynucleotide
atacatattttctatacagggggt

SEQ ID NO: 56 - DNA, 24 bp, Artificial Sequence
  Synthetic polynucleotide
atgattctcttctatataggcagg

SEQ ID NO: 57 - DNA, 24 bp, Artificial Sequence
  Synthetic polynucleotide
aacgtggtctactatacaggagga

SEQ ID NO: 58 - DNA, 24 bp, Artificial Sequence
  Synthetic polynucleotide
taataacccttttatacatgttgg

SEQ ID NO: 59 - DNA, 24 bp, Artificial Sequence
  Synthetic polynucleotide
aacataagcttcgatacagtgagt

SEQ ID NO: 60 - DNA, 24 bp, Artificial Sequence
  Synthetic polynucleotide
tctcattctttctataaagggggc

SEQ ID NO: 61 - DNA, 24 bp, Artificial Sequence
  Synthetic polynucleotide
catatatacttatatacagcgaga

SEQ ID NO: 62 - DNA, 24 bp, Artificial Sequence
  Synthetic polynucleotide
aaccaaatcttcaacacaggtagc

SEQ ID NO: 63 - DNA, 24 bp, Artificial Sequence
  Synthetic polynucleotide
tgtcagtgcttctaaacaagatgg

SEQ ID NO: 64 - DNA, 24 bp, Artificial Sequence
  Synthetic polynucleotide
gaatctcctttttatacaggttgg

SEQ ID NO: 65 - DNA, 24 bp, Artificial Sequence
  Synthetic polynucleotide
atccaagacttctgtacaagagga

SEQ ID NO: 66 - DNA, 24 bp, Artificial Sequence
  Synthetic polynucleotide
atccaattcttccatataggctgg

SEQ ID NO: 67 - DNA, 24 bp, Artificial Sequence
  Synthetic polynucleotide
ttaattagcttctttacatgcggc

SEQ ID NO: 68 - DNA, 24 bp, Artificial Sequence
  Synthetic polynucleotide
tgaccttcattcaatacaggttgg

SEQ ID NO: 69 - DNA, 24 bp, Artificial Sequence
  Synthetic polynucleotide
ttcgttgcctttgatacagggagt

SEQ ID NO: 70 - DNA, 24 bp, Artificial Sequence
  Synthetic polynucleotide
ccggaagtcatctctacaggatgg

SEQ ID NO: 71 - DNA, 24 bp, Artificial Sequence
  Synthetic polynucleotide
agtgttggattctatactggaggc

SEQ ID NO: 72 - DNA, 24 bp, Artificial Sequence
  Synthetic polynucleotide
cgacaggcattccatacaggagga

SEQ ID NO: 73 - DNA, 24 bp, Artificial Sequence
  Synthetic polynucleotide
ttgaaacacttaaatacaggaaga

SEQ ID NO: 74 - DNA, 24 bp, Artificial Sequence
  Synthetic polynucleotide
agctgggtcttctatacacatggg

SEQ ID NO: 75 - DNA, 24 bp, Artificial Sequence
  Synthetic polynucleotide
tgcgttgtcttttatataggcgga

SEQ ID NO: 76 - DNA, 24 bp, Artificial Sequence
  Synthetic polynucleotide
cgacgaaactcatatacaggaggt

SEQ ID NO: 77 - DNA, 24 bp, Artificial Sequence
  Synthetic polynucleotide
taaaattagttctataaaggaaga

SEQ ID NO: 78 - DNA, 24 bp, Artificial Sequence
  Synthetic polynucleotide
tctataggctactatacatgaagg

SEQ ID NO: 79 - DNA, 24 bp, Artificial Sequence
  Synthetic polynucleotide
caaaatatcttttatacaaggaga

SEQ ID NO: 80 - DNA, 24 bp, Artificial Sequence
  Synthetic polynucleotide
gtggtcgccttctttacaagaaga

SEQ ID NO: 81 - DNA, 24 bp, Artificial Sequence
  Synthetic polynucleotide
aaggtgcacttttatacaagctgg

SEQ ID NO: 82 - DNA, 24 bp, Artificial Sequence
  Synthetic polynucleotide
tattttttcttcgatatagggaga

SEQ ID NO: 83 - DNA, 24 bp, Artificial Sequence
  Synthetic polynucleotide
tgctggagcgtctacacagggcgg

SEQ ID NO: 84 - DNA, 24 bp, Artificial Sequence
  Synthetic polynucleotide
ttccatttcttgtataaaggtagt

SEQ ID NO: 85 - DNA, 24 bp, Artificial Sequence
  Synthetic polynucleotide
atgtgcagcttctaaacagcacgg

SEQ ID NO: 86 - DNA, 24 bp, Artificial Sequence
  Synthetic polynucleotide
aaacggatcttctgtacagcgaga

SEQ ID NO: 87 - DNA, 24 bp, Artificial Sequence
  Synthetic polynucleotide
tatatatacatatatacaggtagg

SEQ ID NO: 88 - DNA, 24 bp, Artificial Sequence
  Synthetic polynucleotide
atgataaacttctacactggaagg

SEQ ID NO: 89 - DNA, 24 bp, Artificial Sequence
  Synthetic polynucleotide
gatcagttcttttatgcaggtaga

SEQ ID NO: 90 - DNA, 24 bp, Artificial Sequence
  Synthetic polynucleotide
cttaggtccttctattaaggaaga

SEQ ID NO: 91 - DNA, 24 bp, Artificial Sequence
  Synthetic polynucleotide
aatttcaccttcagtacaggtaga

SEQ ID NO: 92 - DNA, 24 bp, Artificial Sequence
  Synthetic polynucleotide
tgctgtttcttctgtagaggaggt

SEQ ID NO: 93 - DNA, 24 bp, Artificial Sequence
  Synthetic polynucleotide
cacaacattttttatacagggagt

SEQ ID NO: 94 - DNA, 24 bp, Artificial Sequence
  Synthetic polynucleotide
cacaagtgctgcaatacaggagga

SEQ ID NO: 95 - DNA, 24 bp, Artificial Sequence
  Synthetic polynucleotide
tgcagattcttctatgcagtcagc

SEQ ID NO: 96 - DNA, 24 bp, Artificial Sequence
  Synthetic polynucleotide
taaaggatcttctatacggcgtgg

SEQ ID NO: 97 - DNA, 24 bp, Artificial Sequence
  Synthetic polynucleotide
gtagctcacctctatacaggtggt

SEQ ID NO: 98 - DNA, 24 bp, Artificial Sequence
  Synthetic polynucleotide
cacggtcccttctataaagatggt

SEQ ID NO: 99 - DNA, 24 bp, Artificial Sequence
  Synthetic polynucleotide
gagcagggcttctaaacaagatgg

SEQ ID NO: 100 - DNA, 24 bp, Artificial Sequence
  Synthetic polynucleotide
catattatctcctatacacgaggc

SEQ ID NO: 101 - DNA, 24 bp, Artificial Sequence
  Synthetic polynucleotide
tcagaatatttctatataggaagt

SEQ ID NO: 102 - DNA, 24 bp, Artificial Sequence
  Synthetic polynucleotide
acaaatacattatatacagggagt

SEQ ID NO: 103 - DNA, 24 bp, Artificial Sequence
  Synthetic polynucleotide
atgggttccttcttcacaggtaga

SEQ ID NO: 104 - DNA, 24 bp, Artificial Sequence
  Synthetic polynucleotide
ttccgtttcttcaatagaggagga

SEQ ID NO: 105 - DNA, 24 bp, Artificial Sequence
  Synthetic polynucleotide
aaccttgtcttcaatccaggcggc

SEQ ID NO: 106 - DNA, 24 bp, Artificial Sequence
  Synthetic polynucleotide
aagagttctttctatacatgtaga

SEQ ID NO: 107 - DNA, 24 bp, Artificial Sequence
  Synthetic polynucleotide
tgatcaatcttctacgcagggaga

SEQ ID NO: 108 - DNA, 24 bp, Artificial Sequence
  Synthetic polynucleotide
tgtagcttcctctatacatgctgg

SEQ ID NO: 109 - DNA, 24 bp, Artificial Sequence
  Synthetic polynucleotide
gtgttgttcttatatacaggaggc

SEQ ID NO: 110 - DNA, 24 bp, Artificial Sequence
  Synthetic polynucleotide
atttcacacttttatacaagagga

SEQ ID NO: 111 - DNA, 24 bp, Artificial Sequence
  Synthetic polynucleotide
aacgaagtcttctatacaaagaga

SEQ ID NO: 112 - DNA, 24 bp, Artificial Sequence
  Synthetic polynucleotide
cacggtcccttctataaagatggt

SEQ ID NO: 113 - DNA, 24 bp, Artificial Sequence
  Synthetic polynucleotide
atcggtttcttctattcatgtagt

SEQ ID NO: 114 - DNA, 24 bp, Artificial Sequence
  Synthetic polynucleotide
gagcaacgttactatacagggagt

SEQ ID NO: 115 - DNA, 24 bp, Artificial Sequence
  Synthetic polynucleotide
atatatattttctataaaggtagt

SEQ ID NO: 116 - DNA, 24 bp, Artificial Sequence
  Synthetic polynucleotide
gccattggcttctaaacagctaga

SEQ ID NO: 117 - DNA, 24 bp, Artificial Sequence
  Synthetic polynucleotide
gtaatatgcttttatataggttgg

SEQ ID NO: 118 - DNA, 24 bp, Artificial Sequence
  Synthetic polynucleotide
tgcgctaacttctataaaggaggg

SEQ ID NO: 119 - DNA, 24 bp, Artificial Sequence
  Synthetic polynucleotide
aacacgctttccctagtctgtagc

SEQ ID NO: 120 - DNA, 24 bp, Artificial Sequence
  Synthetic polynucleotide
gcctatgcttcactagtcacaggc

SEQ ID NO: 121 - DNA, 24 bp, Artificial Sequence
  Synthetic polynucleotide
tcacttctttccctactcatgggc

SEQ ID NO: 122 - DNA, 24 bp, Artificial Sequence
  Synthetic polynucleotide
agggcaatttccccaatcagttgg

SEQ ID NO: 123 - DNA, 24 bp, Artificial Sequence
  Synthetic polynucleotide
gttttcgttgccctcgtcagccgg

SEQ ID NO: 124 - DNA, 24 bp, Artificial Sequence
  Synthetic polynucleotide
ttgaccttttcctgagtcagaaga

SEQ ID NO: 125 - DNA, 24 bp, Artificial Sequence
  Synthetic polynucleotide
tgatacagtaccccagtcagtggc

SEQ ID NO: 126 - DNA, 24 bp, Artificial Sequence
  Synthetic polynucleotide
aatgatatttccccattcagtgga

SEQ ID NO: 127 - DNA, 24 bp, Artificial Sequence
  Synthetic polynucleotide
tctccagcttcactagacagtggt

SEQ ID NO: 128 - DNA, 24 bp, Artificial Sequence
  Synthetic polynucleotide
agcagctgtttcatagtcagcgga

SEQ ID NO: 129 - DNA, 24 bp, Artificial Sequence
  Synthetic polynucleotide
acaaaatcttccctagctagttgg

SEQ ID NO: 130 - DNA, 24 bp, Artificial Sequence
  Synthetic polynucleotide
aagttgagttctcaagtcagcggt

SEQ ID NO: 131 - DNA, 24 bp, Artificial Sequence
  Synthetic polynucleotide
gctatgcattcccaattcagaaga

SEQ ID NO: 132 - DNA, 24 bp, Artificial Sequence
  Synthetic polynucleotide
tcctgcgtttccgtagtcaagggt

SEQ ID NO: 133 - DNA, 24 bp, Artificial Sequence
  Synthetic polynucleotide
catctggtttctcaagtcagcggt

SEQ ID NO: 134 - DNA, 24 bp, Artificial Sequence
  Synthetic polynucleotide
aaaggtgattctctagtcacgtgg

SEQ ID NO: 135 - DNA, 24 bp, Artificial Sequence
  Synthetic polynucleotide
cattttatttctatagtcagaagt

SEQ ID NO: 136 - DNA, 24 bp, Artificial Sequence
  Synthetic polynucleotide
atggattattccttagtcattggt

SEQ ID NO: 137 - DNA, 24 bp, Artificial Sequence
  Synthetic polynucleotide
cacatttgttccattgtcagttgg

SEQ ID NO: 138 - DNA, 24 bp, Artificial Sequence
  Synthetic polynucleotide
ccaaatttttccccagtgagggga

SEQ ID NO: 139 - DNA, 24 bp, Artificial Sequence
  Synthetic polynucleotide
tcagttctttccctagtatgtagt

SEQ ID NO: 140 - DNA, 24 bp, Artificial Sequence
  Synthetic polynucleotide
cacatttgttccattgtcagttgg

SEQ ID NO: 141 - DNA, 24 bp, Artificial Sequence
  Synthetic polynucleotide
actcggatttccctggtctggagc

SEQ ID NO: 142 - DNA, 24 bp, Artificial Sequence
  Synthetic polynucleotide
tagtaatatttactagtcagtggg

SEQ ID NO: 143 - DNA, 24 bp, Artificial Sequence
  Synthetic polynucleotide
tctttgaattcccttttcagaagt

SEQ ID NO: 144 - DNA, 24 bp, Artificial Sequence
  Synthetic polynucleotide
gatcactttttcccagtcagtaga

SEQ ID NO: 145 - DNA, 24 bp, Artificial Sequence
  Synthetic polynucleotide
gttgaaatttctctagtcaatggt

SEQ ID NO: 146 - DNA, 24 bp, Artificial Sequence
  Synthetic polynucleotide
cttgacttttccctagttcgtaga

SEQ ID NO: 147 - DNA, 24 bp, Artificial Sequence
  Synthetic polynucleotide
aactcttgttccctggccagcagt

SEQ ID NO: 148 - DNA, 24 bp, Artificial Sequence
  Synthetic polynucleotide
gtaatccgttcagtagtcagagga

SEQ ID NO: 149 - DNA, 24 bp, Artificial Sequence
  Synthetic polynucleotide
tgccgtctgaccctagtcaggagc

SEQ ID NO: 150 - DNA, 24 bp, Artificial Sequence
  Synthetic polynucleotide
ccagtgctttccctagtccgtggt

SEQ ID NO: 151 - DNA, 24 bp, Artificial Sequence
  Synthetic polynucleotide
atatataaatccctcgtcagggga

SEQ ID NO: 152 - DNA, 24 bp, Artificial Sequence
  Synthetic polynucleotide
aaataaattgcccgagtcagtgga

SEQ ID NO: 153 - DNA, 24 bp, Artificial Sequence
  Synthetic polynucleotide
agtgtattttccctcctcagggga

SEQ ID NO: 154 - DNA, 24 bp, Artificial Sequence
  Synthetic polynucleotide
gagggagatgccctggtcaggagc

SEQ ID NO: 155 - DNA, 24 bp, Artificial Sequence
  Synthetic polynucleotide
aaatgtcattccatagccagtgga

SEQ ID NO: 156 - DNA, 24 bp, Artificial Sequence
  Synthetic polynucleotide
ctttctaattccctcatcaggcgg

SEQ ID NO: 157 - DNA, 24 bp, Artificial Sequence
  Synthetic polynucleotide
accggtctttccatagtcaagaga

SEQ ID NO: 158 - DNA, 24 bp, Artificial Sequence
  Synthetic polynucleotide
cgactagtttccccagtctgacgg

SEQ ID NO: 159 - DNA, 24 bp, Artificial Sequence
  Synthetic polynucleotide
ttctcatttttcctattcagagga

SEQ ID NO: 160 - DNA, 24 bp, Artificial Sequence
  Synthetic polynucleotide
tccaagctttctcttgtcagctgg

SEQ ID NO: 161 - DNA, 24 bp, Artificial Sequence
  Synthetic polynucleotide
gtcagatgtgccctagtcagcgga

SEQ ID NO: 162 - DNA, 24 bp, Artificial Sequence
  Synthetic polynucleotide
ttgattcggtccctcgtcaggaga

SEQ ID NO: 163 - DNA, 24 bp, Artificial Sequence
  Synthetic polynucleotide
tcctgtctttccgtggtcagtggg

SEQ ID NO: 164 - DNA, 24 bp, Artificial Sequence
  Synthetic polynucleotide
tcattctattcctttgtcagtaga

SEQ ID NO: 165 - DNA, 24 bp, Artificial Sequence
  Synthetic polynucleotide
cacatttgttccattgtcagttgg

SEQ ID NO: 166 - DNA, 24 bp, Artificial Sequence
  Synthetic polynucleotide
gacaaaccttccttggtcagcagc

SEQ ID NO: 167 - DNA, 24 bp, Artificial Sequence
  Synthetic polynucleotide
gtactctactcccaagtcagcgga

SEQ ID NO: 168 - DNA, 24 bp, Artificial Sequence
  Synthetic polynucleotide
attggctcttctctcgtcagtagg

SEQ ID NO: 169 - DNA, 24 bp, Artificial Sequence
  Synthetic polynucleotide
ttccaagtttacctagccagaaga

SEQ ID NO: 170 - DNA, 23 bp, Artificial Sequence
  Synthetic polynucleotide
ccgttaacccgagctcacctgac

SEQ ID NO: 171 - DNA, 23 bp, Artificial Sequence
  Synthetic polynucleotide
ggcaattgggctcgagtggactg